(19) United States
(12) Patent Application Publication
(10) Pub. No.: US 2023/0140123 A1
(43) Pub. Date: May 4, 2023

(54) SYSTEMS AND METHODS FOR CLASSIFYING AND TREATING HOMOLOGOUS REPAIR DEFICIENCY CANCERS

(71) Applicant: Foundation Medicine, Inc., Cambridge, MA (US)

(72) Inventors: Ethan SOKOL, Somerville, MA (US); Jay MOORE, New York, NY (US); Justin NEWBERG, Cambridge, MA (US); Dexter JIN, Stoneham, MA (US); Kuei-Ting CHEN, Malden, MA (US); Russell MADISON, Encinitas, CA (US)

(73) Assignee: Foundation Medicine, Inc., Cambridge, MA (US)

(21) Appl. No.: 17/899,470

(22) Filed: Aug. 30, 2022

Related U.S. Application Data
(63) Continuation-in-part of application No. PCT/US2022/073167, filed on Jun. 24, 2022.
(60) Provisional application No. 63/215,281, filed on Jun. 25, 2021.

(51) Int. Cl. G16B 20/20; C12Q 1/6886; G16B 20/10; G16B 40/20

(52) U.S. Cl. CPC G16B 20/20; C12Q 1/6886; G16B 20/10; G16B 40/20

(57) ABSTRACT

Described herein are methods, devices, and systems for identifying a subset of a plurality of features, using one or more feature importance metrics, for training and using a homologous repair deficiency (HRD) classification model. Further described are methods, devices, and systems for classifying a tumor of a cancer, such as pancreatic cancer, as likely HRD positive or likely HRD negative, and for calling the tumor as HRD positive or HRD negative. Also described herein are methods of treating a tumor of a cancer, such as pancreatic cancer, based on the classifications

### CROSS-REFERENCE TO RELATED APPLICATIONS

This application is a continuation-in-part of International Application No. PCT/US2022/073167, filed on Jun. 24, 2022, which claims the priority benefit of U.S. Provisional Application No. 63/215,281, filed on Jun. 25, 2021, titled “SYSTEM AND METHOD OF CLASSIFYING HOMOLOGOUS REPAIR DEFICIENCY”, the contents of which are incorporated herein by reference for all purposes.

### FIELD OF THE INVENTION

Described herein are methods, devices, and systems for selecting features for a homologous repair deficiency (HRD) model, assessing tumors using the HRD model, and treating a tumor based on the assessment. Also described are methods of treating HRD-positive cancers, including those HRD-positive cancers identified using the methods, devices, and systems described herein.

### BACKGROUND OF THE INVENTION

Copy number aberrations involve the deletion or amplification of large contiguous segments of the genome, and are common mutations in cancer. Certain copy number aberrations are associated with an inability to repair the genome by homologous recombination repair mechanisms, termed homologous repair deficiency (HRD). To identify some tumors with HRD, it is possible to sequence mutations in genes involved in the homologous repair pathway. Alternatively, it is possible to detect genomic scarring, which is the physical consequence of HRD, regardless of its cause.

Tumor genomes exhibiting HRD are associated with sensitivity to certain drugs, such as platinum chemotherapies or poly(ADP)-ribose polymerase (PARP) inhibitors. However, certain tumors remain difficult to classify as HRD positive. Thus, there remains a need to classify tumors of cancer, such as pancreatic, breast, or prostate cancer, where it is especially important, as HRD positive or HRD negative, so that appropriate treatments can be selected and administered to subjects. In the past, techniques for identifying HRD have suffered from inaccuracy and inefficiencies that have not allowed them to be used in practice. One reason for this is that feature selection techniques are currently insufficient to be able to accurately determine the HRD status of a sample in order to identify (e.g., classify) said tumors as HRD positive or HRD negative efficiently and accurately, e.g., due to overfitting. Another reason for this is that determining which features to identify to accurately determine the HRD status may also be a challenge. Accordingly, there is a need techniques and systems that accurately and efficiently select a subset of features from a plurality of features that can be used train a model for performing said identification.

### SUMMARY OF THE INVENTION

Described herein are methods for characterizing the status of a tumor or cancer as homologous recombination deficiency (HRD)-positive or HRD-negative. The methods may include the use of a trained HRD model to characterize the cancer or tumor. Further described herein are methods of identifying and/or treating a subject having a cancer or tumor, or selecting a treatment or identifying one or more treatment options for a subject having a cancer or tumor, with a therapy that includes a platinum-based chemotherapeutic agent (for example, FOLFIRINOX) or a poly(ADP-ribose) polymerase (PARP) inhibitor, based on the HRD status of a cancer or tumor sample obtained from the subject. Also described are methods of predicting survival of a subject having cancer; methods of monitoring, evaluating, or screening a subject having a cancer; and methods of stratifying a subject with a cancer for treatment with a therapy comprising a platinum-based chemotherapeutic agent or a PARP inhibitor

Described herein are methods comprising: providing a genome obtained from a tumor of a subject; optionally, ligating one or more adapters onto the genome; amplifying nucleic acid molecules from the genome; capturing nucleic acid molecules from the amplified genome, wherein the captured nucleic acid molecules are captured by hybridization to one or more bait molecules; deriving, from the captured nucleic acid molecules, a set of input features; inputting, by one or more processors, the set of input features into a trained homologous recombination deficiency (HRD) model to identify the tumor as HRD-positive or HRD-negative using the trained HRD model, wherein the model is trained by: determining one or more feature importance metrics associated with each feature of a plurality of features, identifying a subset of features in the plurality of features using the one or more feature importance metrics, and training, by the one or more processors, the HRD model based on the identified subset of features; and classifying, by the one or more processors, using the trained HRD model, the tumor as HRD-positive or HRD-negative.

Further described herein are methods comprising: receiving, by one or more processors, a plurality of features; identifying, by the one or more processors, a subset of features in the plurality of features using one or more feature importance metrics; and training, by the one or more processors, a homologous recombination deficiency (HRD) model based on the identified subset of the plurality of features, wherein the HRD model is configured to receive sample data associated with a genome of a tumor in a subject and identify the tumor in the subject as HRD-positive or HRD-negative using the sample data.

Further described herein are methods comprising: receiving, by one or more processors, sample data associated with a genome of a tumor in a subject; inputting, by the one or more processors, the sample data into a trained homologous recombination deficiency (HRD) model, wherein the HRD model is trained by: determining one or more feature importance metrics associated with each feature of a plurality of features, identifying a subset of features in the plurality of features using the one or more feature importance metrics, and training, by the one or more processors, the HRD model based on the identified subset of features; and classifying, by the one or more processors, using the trained HRD model, the tumor as HRD-positive or HRD-negative.

In some embodiments of the described methods, the plurality of features comprises one or more copy number features, one or more short variant features, or a combination thereof. In some embodiments of the described methods, the one or more feature importance metrics comprise one or more of a Chi-Square test, analysis of variance (ANOVA), random forest, or gradient boosting.

In some embodiments of the described methods, identifying the subset of features in the plurality of features comprises: obtaining, by the one or more processors, one or more feature rankings according to the one or more feature importance metrics; and selecting, by the one or more processors, the subset of the plurality of features based on one or more feature rankings.

In some embodiments of the described methods, identifying the subset of the plurality of features comprises: (a) obtaining, by one or more processors, a feature ranking of the plurality of features according to a feature importance metric; (b) obtaining, by the one or more processors, a new feature set by adding one or more features from the plurality of features to an existing feature set based on the feature ranking; (c) training, by the one or more processors, a new HRD model using the new feature set; (d) evaluating, by the one or more processors, the trained new HRD model to obtain an evaluation result; and (e) storing, by the one or more processors, the evaluation result associated with the new HRD model and the new feature set; (f) repeating, by the one or more processors, steps (b)-(e) to obtain a plurality of evaluation results until a condition is met; and (g) selecting, by the one or more processors, the subset of the plurality of features based on the plurality of evaluation results.

In some embodiments of the described methods, the trained HRD model is a classification model, the method further comprising: receiving new sample data associated with a genome of a tumor in a new subject, wherein the new sample data is related to the subset of the plurality of features; providing the new sample data to the trained HRD classification model to produce a classification result of HRD-positive or HRD-negative; and outputting the classification result. In some embodiments, the classification result comprises at least one of a HRD-positive likelihood score and a HRD-negative likelihood score. In some embodiments, the method comprises recording, in a digital electronic file associated with the new subject, at least one of the HRD-positive likelihood score and the HRD-negative likelihood score. In some embodiments, the method comprises recording in a digital electronic file associated with the new subject that the tumor is HRD positive based on the HRD positive likelihood score or a designation that the tumor is HRD negative based on the HRD negative likelihood score.

In some embodiments of the described methods, the HRD model is a classification model, a regression model, a neural network, or any combination thereof. In some embodiments, the method comprises recording, in a digital electronic file associated with the new subject, at least one of the HRD-positive likelihood score and the HRD-negative likelihood score. In some embodiments, the method comprises recording in a digital electronic file associated with the new subject that the tumor is HRD positive based on the HRD positive likelihood score or a designation that the tumor is HRD negative based on the HRD negative likelihood score.

In some embodiments of the described methods, the plurality of features comprise at least one of a segment minor allele frequency (segMAF) feature, a number of sequencing reads feature, a segment size feature, a breakpoint count per x megabases feature, a change point copy number feature, a segment copy number feature, a breakpoint count per chromosome arm feature, or a number of segments with oscillating copy number feature. In some embodiments of the described methods, at least one of the plurality of features is assessed across the centromeric portion of the genome. In some embodiments of the described methods, at least one of the plurality of features is assessed across the telomeric portion of the genome.

In some embodiments of the described methods, at least one of the plurality of features is assessed across both the centromeric and telomeric portions of the genome.

In some embodiments of the described methods, the plurality of features comprise a breakpoint count per x megabases feature, wherein the breakpoint count per x megabases feature is based on the number of breakpoints appearing in windows of x megabases in length across the genome. In some embodiments, breakpoint count per x megabases feature is assessed across: (i) the telomeric portion of the genome; (ii) the centromeric portion of the genome; or (iii) both the telomeric portion and the centromeric portion of the genome. In some embodiments, x is between about 1 and about 100 megabases. In some embodiments, x is about 10 megabases, about 25 megabases, about 50 megabases, or about 100 megabases. In some embodiments, the breakpoint count per x megabases feature is a binned feature.

In some embodiments of the described methods, the plurality of features comprise a change point copy number feature, wherein the change point copy number is based on the absolute difference in copy number between adjacent genome segments across the genome of the tumor of the subject. In some embodiments, the change point copy number feature is derived from ploidy-normalized copy number data. In some embodiments, change point copy number feature is assessed across: (i) the telomeric portion of the genome; (ii) the centromeric portion of the genome; or (iii) both the telomeric portion and the centromeric portion of the genome. In some embodiments, the change point copy number feature is a binned feature.

In some embodiments of the described methods, the plurality of features comprise a segment copy number feature, wherein segment copy number is based on the copy number of each genome segment. In some embodiments, the segment copy number feature is assessed across: (i) the telomeric portion of the genome; (ii) the centromeric portion of the genome; or (iii) both the telomeric portion and the centromeric portion of the genome. In some embodiments, the segment copy number feature is derived from ploidy-normalized copy number data. In some embodiments, the segment copy number feature is a binned feature.

In some embodiments of the described methods, the plurality of features comprise a breakpoint count per chromosome arm feature in the genome of the tumor of the subject. In some embodiments, the breakpoint count per chromosome arm feature is assessed across: (i) the telomeric portion of the genome; (ii) the centromeric portion of the genome; or (iii) both the telomeric portion and the centromeric portion of the genome. In some embodiments, the breakpoint count per chromosome arm feature is a binned feature.

In some embodiments of the described methods, the plurality of features comprise a number of segments with oscillating copy number feature. In some embodiments, the number of segments with oscillating copy number feature is based on the number of repeated alternating segments between two copy numbers across the genome of the tumor of the subject. In some embodiments, number of segments with oscillating copy number feature is assessed across: (i) the telomeric portion of the genome; (ii) the centromeric portion of the genome; or (iii) both the telomeric portion and the centromeric portion of the genome. In some embodiments, the number of segments with oscillating copy number feature is a binned feature.

In some embodiments of the described methods, the one or more copy number features comprise a segment minor allele frequency (segMAF) feature, wherein segMAF is based on the minor allele frequency at heterozygous single nucleotide polymorphisms. In some embodiments, segMAF is assessed across: (i) the telomeric portion of the genome; (ii) the centromeric portion of the genome; or (iii) both the telomeric portion and the centromeric portion of the genome. In some embodiments, the segMAF feature is a binned feature.

In some embodiments of the described methods, the one or more copy number features comprise a number of sequencing reads feature. In some embodiments, the number of sequencing reads feature is a binned feature.

In some embodiments of the described methods, the plurality of features further comprise a measure of genome-wide loss of heterozygosity of the genome of the tumor of the subject.

In some embodiments of the described methods, the plurality of features comprise one or more short variant features. In some embodiments, the one or more short variant features comprise at least one of a deletions in microhomology or repetitive regions feature and a mutational signature derived from two or more short variant features. In some embodiments, the deletions in microhomology or repetitive regions feature are deletions of at least 5 basepairs.

In some embodiments of the described methods, training the HRD model comprises: receiving, by the one or more processors, an HRD-positive training dataset, wherein the HRD-positive training dataset comprises a plurality of features associated with an HRD-positive tumor and an HRD-positive label; receiving, by the one or more processors, an HRD-negative training dataset, wherein the HRD-negative training dataset comprises a plurality of features associated with an HRD-negative tumor and an HRD-negative label; training, by the one or more processors, the HRD model using the HRD-positive training dataset and the HRD-negative training dataset. In some embodiments, training comprises using a HRD-positive training dataset and an HRD-negative training dataset. In some embodiments, the method comprises balancing, by the one or more processors, the HRD-positive training dataset and the HRD-negative training dataset prior to training the HRD model.

In some embodiments of the described methods, the method further comprises testing, by the one or more processors, the trained model using a HRD-positive testing dataset comprising a HRD-positive control derived from a genome sequence comprising loss-of-function mutations in BRCA1, BRCA2, both BRCA1 and BRCA2, or biallelic mutations of BRCA1 and BRCA2. In some embodiments, training comprises using a HRD-positive training dataset and an HRD-negative training dataset. In some embodiments, the method comprises balancing, by the one or more processors, the HRD-positive training dataset and the HRD-negative training dataset prior to training the HRD model.

In some embodiments of the described methods, the method further comprises testing, by the one or more processors, the trained model using a HRD-positive testing dataset comprising a HRD-positive control derived from a genome sequence comprising loss-of-function mutations in at least one of ATM, BARD1, BRIP1, CDK12, CHEK1, CHEK2, FANCL, PALB2, RAD51B, RAD51C, RAD51D, or RAD45L. In some embodiments, training comprises using a HRD-positive training dataset and an HRD-negative training dataset. In some embodiments, the method comprises balancing, by the one or more processors, the HRD-positive training dataset and the HRD-negative training dataset prior to training the HRD model.

In some embodiments of the described methods, the method further comprises testing, by the one or more processors, the trained model using a HRD-negative testing dataset comprising a HRD-negative training dataset comprising a HRD-negative control derived from a consensus human genome sequence. In some embodiments, training comprises using a HRD-positive training dataset and an HRD-negative training dataset. In some embodiments, the method comprises balancing, by the one or more processors, the HRD-positive training dataset and the HRD-negative training dataset prior to training the HRD model.

In some embodiments of the described methods, the tumor in the subject is a prostate cancer, non-small cell lung cancer (NSCLC), colorectal cancer (CRC), ovarian cancer, breast cancer, or pancreatic cancer.

In some embodiments of the described methods, training the HRD model comprises fitting the HRD model to sample data associated with ovarian cancer, non-small cell lung cancer (NSCLC), colorectal cancer (CRC), breast cancer, pancreatic cancer, or prostate cancer, wherein the sample data comprises the subset of the plurality of features.

In some embodiments of the described methods, the tumor is obtained from a sample that is a solid tissue biopsy sample. In some embodiments, the solid tissue biopsy sample is a formalin-fixed paraffin-embedded (FFPE) sample. In some embodiments of the described methods, the tumor is obtained from a sample that is a liquid biopsy sample comprising circulating tumor DNA (ctDNA). In some embodiments of the described methods, the tumor is obtained from a sample that is a liquid biopsy sample comprising cell-free DNA (cfDNA).

In some embodiments of the described methods, the method further comprises: determining, identifying, or applying the output of the tumor as HRD-positive or HRD-negative as a diagnostic value associated with the patient. In some embodiments of the described methods, the method further comprises generating a genomic profile for the subject based on the output of the tumor as HRD-positive or HRD-negative. In some embodiments, the method further comprises administering an anti-cancer agent or applying an anti-cancer treatment to the subject based on the generated genomic profile. In some embodiments of the described methods, the output of the tumor as HRD-positive or HRD-negative is used in generating a genomic profile for the subject. In some embodiments of the described methods, the output of the tumor as HRD-positive or HRD-negative is used in making suggested treatment decisions for the subject. In some embodiments of the described methods, the output of the tumor as HRD-positive or HRD-negative is used in applying or administering a treatment to the subject.

In some embodiments of the described methods, the HRD model is a machine learning model.

In some embodiments of the described methods, the subject has a cancer, is at risk of having a cancer, or is suspected of having a cancer.

Further described herein are methods of treating cancer in a subject, comprising: (a) identifying the tumor as HRD-positive or HRD-negative according to any method described above; (b) administering to the subject a therapeutically effective amount of a drug effective in a HRD positive tumor if the tumor of the cancer is assessed as HRD positive. In some embodiments, the drug effective in a HRD positive tumor is a platinum-based drug or a PARP inhibitor. In some embodiments, the method comprises administering to the subject a therapeutically effective amount of a drug that is not a platinum-based drug or a PARP inhibitor if the tumor is assessed as HRD negative.

Further described herein are methods for selecting a therapy for a cancer in a subject, the method comprising: (a) assessing a tumor of the cancer as HRD-positive or HRD-negative according to any method described above; (b) selecting a therapy that is effective in a HRD positive tumor if the cancer is assessed as HRD positive. In some embodiments, the method comprises selecting a therapy that is not a platinum-based drug or a PARP inhibitor if the tumor is assessed as HRD negative. In some embodiments, the therapy that is effective in a HRD positive tumor is a platinum-based drug or a PARP inhibitor.

Further described herein are computer systems, comprising: one or more processors; a memory; and one or more programs, wherein the one or more programs are stored in the memory and configured to be executed by the one or more processors, the one or more programs including instructions for: performing any one of the methods described above.

Further described herein are non-transitory computer-readable storage media storing one or more programs, the one or more programs comprising instructions, which when executed by one or more processors of an electronic device, cause the electronic device to perform the any one of the methods described above.

Also described herein is a method for identifying an individual having a cancer for treatment with a therapy comprising a platinum-based chemotherapeutic agent or a PARP inhibitor, comprising: determining a homologous recombination deficient (HRD) status of a sample obtained from the subject, and identifying the subject for therapy comprising the platinum-based chemotherapeutic agent or the PARP inhibitor if the HRD status of the sample is identified as HRD-positive.

Further described herein is a method of treating a subject having a cancer with a therapy comprising a platinum-based chemotherapeutic agent or a PARP inhibitor, comprising: determining a homologous recombination deficient (HRD) status of a sample obtained from the subject, and administering the platinum-based chemotherapeutic agent or the PARP inhibitor to the subject if the HRD status of the sample is determined to be HRD-positive.

Also described herein is a method of selecting a treatment for a subject having a cancer, comprising determining a homologous recombination deficient (HRD) status of a sample obtained from the subject, wherein a HRD-positive status identifies the subject as one who may benefit from treatment with a therapy comprising a platinum-based chemotherapeutic agent or a PARP inhibitor.

Further described is a method of identifying one or more treatment options for a subject having a cancer, comprising: determining a homologous recombination deficient (HRD) status of a sample obtained from the subject, and generating a report comprising one or more treatment options identified for the subject based at least in part on the HRD status for the sample, wherein a HRD-positive status in the sample identifies the subject as one who may benefit from treatment with a therapy comprising a platinum-based chemotherapeutic agent or a PARP inhibitor.

Also described herein is a method of predicting survival of a subject having a cancer, comprising acquiring knowledge of a homologous recombination deficient (HRD) status of a sample obtained from the subject, wherein the HRD status for the sample obtained from the subject is a HRD-positive status, and wherein responsive to the acquisition of said knowledge, the subject is predicted to have longer survival when treated with a therapy comprising a platinum-based chemotherapeutic agent or a PARP inhibitor, as compared to treatment comprising a therapy without the platinum-based chemotherapeutic agent or the PARP inhibitor.

Further described is a method of monitoring, evaluating, or screening a subject having a cancer, comprising acquiring knowledge of a homologous recombination deficient (HRD) status of a sample obtained from the subject, wherein the HRD status for the sample obtained from the subject is a HRD-positive status, and wherein responsive to the acquisition of said knowledge, the subject is predicted to have longer survival when treated with a therapy comprising a platinum-based chemotherapeutic agent or a PARP inhibitor, as compared to treatment with a therapy without the platinum-based chemotherapeutic agent or the PARP inhibitor.

Further described herein is a method of stratifying a subject with a cancer for a treatment with a therapy comprising a platinum-based chemotherapeutic agent or a PARP inhibitor, comprising acquiring knowledge of a homologous recombination deficient (HRD) status of a sample obtained from the subject, and (a) if the HRD status is a HRD-positive status, identifying the subject as a candidate for receiving the platinum-based chemotherapeutic agent or the PARP inhibitor; or (b) if the HRD status is a HRD-negative status, identifying the subject as a candidate for receiving treatment without the platinum-based chemotherapeutic agent or the PARP inhibitor.

Further described herein is a method of treating a homologous recombination deficient (HRD)-positive cancer in a subject, comprising: identifying the cancer as an HRD-positive cancer, comprising: obtaining genomic data comprising values for a plurality of genomic features for the cancer; inputting, by one or more processors, the genomic data into a trained HRD model configured to characterize the cancer as HRD-positive or HRD-negative based on the genomic data; and characterizing, by the one or more processors, using the trained HRD model, the cancer as HRD-positive; and responsive to identifying the cancer as an HRD-positive cancer, administering to the subject a therapy comprising a platinum-based chemotherapeutic agent or a PARP inhibitor.

Also described herein is a method of selecting a therapy for a cancer in a subject, comprising: identifying the cancer as an HRD-positive cancer or an HRD-negative cancer, comprising: obtaining genomic data comprising values for a plurality of genomic features for the cancer; inputting, by one or more processors, the genomic data into a trained HRD model configured to characterize the cancer as HRD-positive or HRD-negative based on the genomic data; characterizing, by the one or more processors, using the trained HRD model, the cancer as HRD-positive or HRD-negative; and selecting a therapy for the subject based on the cancer being identified as an HRD-positive cancer or an HRD-negative cancer, wherein the selected therapy comprises a platinum-based chemotherapeutic agent or a PARP inhibitor if the cancer is identified as HRD-positive. In some embodiments, the selected therapy does not comprise a platinum-based chemotherapeutic agent or a PARP inhibitor if the cancer is identified as HRD-negative.

In some embodiments of the above methods, the therapy comprising the platinum-based chemotherapeutic agent or the PARP inhibitor comprises the platinum-based chemotherapeutic. In some embodiments, the platinum-based chemotherapeutic agent is cisplatin, oxaliplatin, or carboplatin. In some embodiments, the platinum-based chemotherapeutic agent is oxaliplatin.

In some embodiments, the therapy comprising the platinum-based chemotherapeutic agent or the PARP inhibitor further comprises an immune checkpoint inhibitor.

In some embodiments, the therapy comprising the platinum-based chemotherapeutic agent or the PARP inhibitor further comprises leucovorin, fluorouracil, or both.

In some embodiments, the therapy comprising the platinum-based chemotherapeutic agent or the PARP inhibitor further comprises a topoisomerase inhibitor. In some embodiments, the topoisomerase inhibitor is irinotecan.

In some embodiments, the therapy comprising the platinum-based chemotherapeutic or the PARP inhibitor comprises FOLFIRINOX.

In some embodiments, the therapy comprising the platinum-based chemotherapeutic or the PARP inhibitor comprises the PARP inhibitor. In some embodiments, the PARP inhibitor comprises veliparib, olaparib, talazoparib, iniparib, rucaparib, fluzoparib, or niraparib.

In some embodiments, the cancer is metastatic.

In some embodiments, the cancer is breast cancer, ovarian cancer, prostate cancer, pancreatic cancer, lung cancer, non-small cell lung cancer (NSCLC), colorectal cancer (CRC), uterine cancer, fallopian tube cancer, endometrial cancer, or urothelial cancer.

In some of the above methods, obtaining genomic data for the cancer in the subject comprises: providing a plurality of nucleic acid molecules obtained from the sample, wherein the plurality of nucleic acid molecules comprises a mixture of tumor nucleic acid molecules and non-tumor nucleic acid molecules; optionally, ligating one or more adapters onto one or more nucleic acid molecules from the plurality of nucleic acid molecules; amplifying nucleic acid molecules from the plurality of nucleic acid molecules; optionally, capturing nucleic acid molecules from the amplified nucleic acid molecules, wherein the captured nucleic acid molecules are captured from the amplified nucleic acid molecules by hybridization to one or more bait molecules; and deriving, from the captured nucleic acid molecules, values for the plurality of genomic features for the cancer. In some embodiments, the deriving comprises sequencing the captured portion of the nucleic acid molecules. In some embodiments, the sequencing comprises high-throughput sequencing.

In some embodiments of the above methods, the HRD model is a classification model, a regression model, a neural network, or any combination thereof.

In some embodiments of the above methods, the trained HRD model is a classification model, wherein the characterizing comprises generating a classification result. In some embodiments, the classification result comprises at least one of a HRD-positive likelihood score and a HRD-negative likelihood score.

In some embodiments of the above methods, the plurality of genomic features comprises one or more copy number features, one or more short variant features, or a combination thereof.

In some embodiments of the above methods, the plurality of genomic features comprise at least one of a segment minor allele frequency (segMAF) feature, a number of sequencing reads feature, a segment size feature, a breakpoint count per x megabases feature, a change point copy number feature, a segment copy number feature, a breakpoint count per chromosome arm feature, or a number of segments with oscillating copy number feature.

In some embodiments of the above methods, n at least one of the plurality of genomic features is assessed across the centromeric portion of a genome of the cancer.

In some embodiments of the above methods, at least one of the plurality of genomic features is assessed across the telomeric portion of the genome of the cancer.

In some embodiments of the above methods, at least one of the plurality of genomic features is assessed across both the centromeric and telomeric portions of the genome of the cancer.

In some embodiments of the above methods, the plurality of genomic features comprise a breakpoint count per x megabases feature, wherein the breakpoint count per x megabases feature is based on the number of breakpoints appearing in windows of x megabases in length across the genome of the cancer. In some embodiments, breakpoint count per x megabases feature is assessed across: (i) the telomeric portion of the genome of the cancer; (ii) the centromeric portion of the genome of the cancer; or (iii) both the telomeric portion and the centromeric portion of the genome of the cancer. In some embodiments, x is between about 1 and about 100 megabases. In some embodiments, x is about 10 megabases, about 25 megabases, about 50 megabases, or about 100 megabases. In some embodiments, the breakpoint count per x megabases feature is a binned feature.

In some embodiments of the above methods, the plurality of genomic features comprise a change point copy number feature, wherein the change point copy number is based on the absolute difference in copy number between adjacent genome segments across the genome of the cancer of the subject. In some embodiments, the change point copy number feature is derived from ploidy-normalized copy number data. In some embodiments, the change point copy number feature is assessed across: (i) the telomeric portion of the genome of the cancer; (ii) the centromeric portion of the genome of the cancer; or (iii) both the telomeric portion and the centromeric portion of the genome of the cancer. In some embodiments, the change point copy number feature is a binned feature.

In some embodiments of the above methods, the plurality of genomic features comprise a segment copy number feature, wherein segment copy number is based on the copy number of each genome segment. In some embodiments, the segment copy number feature is assessed across: (i) the telomeric portion of the genome of the cancer; (ii) the centromeric portion of the genome of the cancer; or (iii) both the telomeric portion and the centromeric portion of the genome of the cancer. In some embodiments, the segment copy number feature is derived from ploidy-normalized copy number data. In some embodiments, the segment copy number feature is a binned feature.

In some embodiments of the above methods, the plurality of genomic features comprise a breakpoint count per chromosome arm feature for the genome of the cancer of the subject. In some embodiments, the breakpoint count per chromosome arm feature is assessed across: (i) the telomeric portion of the genome of the cancer; (ii) the centromeric portion of the genome of the cancer; or (iii) both the telomeric portion and the centromeric portion of the genome of the cancer. In some embodiments, the breakpoint count per chromosome arm feature is a binned feature.

In some embodiments of the above methods, the plurality of genomic features comprise a number of segments with oscillating copy number feature. In some embodiments, the number of segments with oscillating copy number feature is based on the number of repeated alternating segments between two copy numbers across the genome of the tumor of the subject. In some embodiments, number of segments with oscillating copy number feature is assessed across: (i) the telomeric portion of the genome of the cancer; (ii) the centromeric portion of the genome of the cancer; or (iii) both the telomeric portion and the centromeric portion of the genome of the cancer. In some embodiments, the number of segments with oscillating copy number feature is a binned feature.

In some embodiments of the above methods, the plurality of genomic features comprises one or more copy number features. In some embodiments, the one or more copy number features comprise a segment minor allele frequency (segMAF) feature, wherein segMAF is based on the minor allele frequency at heterozygous single nucleotide polymorphisms. In some embodiments, the segMAF feature is assessed across: (i) the telomeric portion of the genome of the cancer; (ii) the centromeric portion of the genome of the cancer; or (iii) both the telomeric portion and the centromeric portion of the genome of the cancer. In some embodiments, the segMAF feature is a binned feature. In some embodiments, the one or more copy number features comprise a number of sequencing reads feature. In some embodiments, the number of sequencing reads feature is a binned feature.

In some embodiments of the above methods, the plurality of genomic features comprise a measure of genome-wide loss of heterozygosity of the genome of the tumor of the subject.

In some embodiments of the above methods, the plurality of genomic features comprise one or more short variant features. In some embodiments, the one or more short variant features comprise at least one of a deletions in microhomology or repetitive regions feature and a mutational signature derived from two or more short variant features. In some embodiments, the deletions in microhomology or repetitive regions feature are deletions of at least 5 basepairs.

In some embodiments of the above methods, the HRD model is trained by: determining one or more feature importance metrics associated with each feature of a set of genomic features, identifying a subset of genomic features in the set of genomic features using the one or more feature importance metrics, wherein the subset of genomic features comprises the plurality of genomic features, and training, by the one or more processors, the HRD model based on the identified subset of genomic features. In some embodiments, the one or more feature importance metrics comprise one or more of a Chi-Square test, analysis of variance (ANOVA), random forest, or gradient boosting. In some embodiments, identifying the subset of genomic features comprises: obtaining, by the one or more processors, one or more feature rankings according to the one or more feature importance metrics; and selecting, by the one or more processors, the subset of genomic features based on one or more feature rankings. In some embodiments, identifying the subset of genomic features comprises: (a) obtaining, by one or more processors, a feature ranking of the set of genomic features according to a feature importance metric; (b) obtaining, by the one or more processors, a new set of genomic features by adding one or more additional genomic features an existing set of genomic features based on the feature ranking; (c) training, by the one or more processors, a new HRD model using the new feature set; (d) evaluating, by the one or more processors, the trained new HRD model to obtain an evaluation result; and (e) storing, by the one or more processors, the evaluation result associated with the new HRD model and the new set of genomic features; (0 repeating, by the one or more processors, steps (b)-(e) to obtain a plurality of evaluation results until a condition is met; and (g) selecting, by the one or more processors, the subset of genomic features based on the plurality of evaluation results. In some embodiments, training the HRD model comprises: receiving, by the one or more processors, an HRD-positive training dataset, wherein the HRD-positive training dataset comprises a plurality of features associated with an HRD-positive tumor and an HRD-positive label; receiving, by the one or more processors, an HRD-negative training dataset, wherein the HRD-negative training dataset comprises a plurality of features associated with an HRD-negative tumor and an HRD-negative label; and training, by the one or more processors, the HRD model using the HRD-positive training dataset and the HRD-negative training dataset. In some embodiments, the method further comprises testing, by the one or more processors, the trained model using a HRD-positive testing dataset comprising a HRD-positive control derived from a genome sequence comprising loss-of-function mutations in BRCA1, BRCA2, both BRCA1 and BRCA2, or biallelic mutations of BRCA1 and BRCA2. In some embodiments, the method further comprises testing, by the one or more processors, the trained model using a HRD-positive testing dataset comprising a HRD-positive control derived from a genome sequence comprising loss-of-function mutations in at least one of ATM, BARD1, BRIP1, CDK12, CHEK1, CHEK2, FANCL, PALB2, RAD51B, RAD51C, RAD51D, or RAD45L. In some embodiments, the method further comprises testing, by the one or more processors, the trained model using a HRD-negative testing dataset comprising a HRD-negative training dataset comprising a HRD-negative control derived from a consensus human genome sequence. In some embodiments, the training comprises using a HRD-positive training dataset and an HRD-negative training dataset. In some embodiments, the method further comprises balancing, by the one or more processors, the HRD-positive training dataset and the HRD-negative training dataset prior to training the HRD model. In some embodiments, training the HRD model comprises fitting the HRD model to training sample data associated with ovarian cancer, non-small cell lung cancer (NSCLC), colorectal cancer (CRC), breast cancer, pancreatic cancer, or prostate cancer, wherein the training sample data comprises the subset of genomic features.

In some embodiments of the above methods, the genomic data obtained from a sample that is a solid tissue biopsy sample. In some embodiments, the solid tissue biopsy sample is a formalin-fixed paraffin-embedded (FFPE) sample.

In some embodiments of the above methods, the genomic data is obtained from a sample that is a liquid biopsy sample. In some embodiments, the liquid biopsy sample comprises circulating tumor DNA (ctDNA). In some embodiments, the liquid biopsy sample comprises cell-free DNA (cfDNA). In some embodiments, the liquid biopsy sample comprises cell-free RNA. In some embodiments, the liquid biopsy sample comprises blood, plasma, serum, cerebrospinal fluid, sputum, stool, urine, or saliva. In some embodiments, the liquid biopsy is blood, plasma, or serum.

In some embodiments of the above methods, the HRD model is a machine-learning model.

In some embodiments of the above methods, the genome of the tumor or the genomic data is determined is determined, at least in part, by sequencing. In some embodiments, the sequencing comprises use of a massively parallel sequencing (MPS) technique, whole genome sequencing (WGS), whole exome sequencing, targeted sequencing, direct sequencing, next-generation sequencing (NGS), or a Sanger sequencing technique. In some embodiments, the sequencing comprises: providing a plurality of nucleic acid molecules obtained from the sample, wherein the plurality of nucleic acid molecules comprises a mixture of tumor nucleic acid molecules and non-tumor nucleic acid molecules; optionally, ligating one or more adapters onto one or more nucleic acid molecules from the plurality of nucleic acid molecules; amplifying nucleic acid molecules from the plurality of nucleic acid molecules; capturing nucleic acid molecules from the amplified nucleic acid molecules, wherein the captured nucleic acid molecules are captured from the amplified nucleic acid molecules by hybridization to one or more bait molecules; sequencing, by a sequencer, the captured nucleic acid molecules to obtain a plurality of sequence reads corresponding to one or more genomic loci within a subgenomic interval in the sample. In some embodiments, the adapters comprise one or more of amplification primer sequences, flow cell adapter hybridization sequences, unique molecular identifier sequences, substrate adapter sequences, or sample index sequences. In some embodiments, amplifying nucleic acid molecules comprises performing a polymerase chain reaction (PCR) technique, a non-PCR amplification technique, or an isothermal amplification technique. In some embodiments, the one or more bait molecules comprise one or more nucleic acid molecules, each comprising a region that is complementary to a region of a captured nucleic acid molecule. In some embodiments, the one or more bait molecules each comprise a capture moiety.

In some embodiments, the capture moiety is biotin.

In some embodiments of the above methods, the tumor or cancer in the subject is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments of the above methods, the subject is a human.

In some embodiments of the above methods, the subject has previously been treated with an anti-cancer therapy.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings.

FIG. 1 shows an exemplary process for classifying a tumor of a cancer in a subject as HRD positive (HRD(+)) or HRD negative (HRD(−)).

FIG. 2 shows different types of features that may be evaluated using different feature importance metrics such as ANOVA, random forest, gradient boosting (e.g., XGB), and Chi-Squared.

FIG. 3A shows an exemplary feature overlap analysis.

FIG. 3B shows an exemplary feature overlap analysis.

FIG. 4 shows an exemplary iterative feature selection process.

FIG. 5 shows an example plot of model performances obtained from an exemplary iterative feature selection process.

FIG. 6A shows an exemplary cross-validation process which may be used to evaluate and tune the performance of a model.

FIG. 6B shows an exemplary division of a plurality of data elements into equally-sized subsets.

FIG. 7 shows an exemplary method for training and operating the HRD classification model configured to classify a tumor of a cancer in a subject as HRD positive (HRD(+)) or HRD negative (HRD(−)).

FIG. 8 shows an example of HRD score distributions for different machine learning models using logistic regression, gradient boosting (e.g., XGB), and random forest.

FIG. 9 shows an example model performance in samples stratified by HRD and/or BRCA1/2 mutation status. The left side shows the pool of sample tumors designated “HRD WildType: True” (N=245,050; −1 on the right side of figure), “HRD WildType:False” (N=30,799; 0 on right side of figure) and true HRD-positive samples (biallelic BRCA mutation; N=6,851; 1 on right side of figure).

FIG. 10 shows the example model performance from the subsets of FIG. 9 in different tumor types (breast, ovarian, pancreatic, and prostate cancer). For each tumor type, the subsets correspond to the subsets −1, 0, and 1 of FIG. 9 (i.e., HRD WildType: True, HRD WildType: False, and biallelic BRCA mutation for each cancer, respectively).

FIG. 11 shows an example of a computing device in accordance with one embodiment, which may be used with certain methods described herein.

FIG. 12 provides an overview of a method of training an HRD signature (HRDsig) model to call HRDsig-positive or HRDsig-negative cancers, applying the model to characterize samples with associated clinciopathological data, and evaluating performance of poly (ADP-ribose) polymerase (PARP) inhibitor (PARPi) therapy in ovarian or prostate cancer, according to some embodiments.

FIG. 13A shows the frequency of HRD positive calls in cancer disease groups. HRD, homologous recombination deficiency.

FIG. 13B shows the overlap of HRDsig-positive patients with patients harboring bialleleic BRCA1/2 alterations.

FIG. 13C shows co-occurrence of biallelic gene alterations with HRDsig in BRCA1/2 wt patients.

FIG. 14A shows the frequency of HRDsig positivity in tissue biopsy (y-axis) or liquid biopsy (x-axis) across disease groups.

FIG. 14B shows frequency of HRDsig positivity in samples with BRCA1/2 deletions in tissue and liquid biopsies.

FIGS. 15A-15F show ROC curves comparing HRDsig to % gLOH and GIS scores for all samples (FIG. 15A), prostate cancer (FIG. 15B), pancreatic cancer (FIG. 15C), ovarian cancer (FIG. 15D), breast cancer (FIG. 15E), and all other samples (FIG. 15F). FIG. 15G shows fraction of biallelic samples that were called as HRDsig-positive in all samples, ovarian cancer, prostate cancer, breast cancer, and pancreatic cancer.

FIG. 16A shows Kaplan-Meier curves demonstrating HRDsig as a predictor of response to PARPi treatment in ovarian cancer based on the probability of treatment discontinuation, according to some embodiments.

FIG. 16B shows the results from a multivariate analysis of predictors of response in ovarian cancer, according to some embodiments.

FIG. 17A shows Kaplan-Meier curves demonstrating HRDsig as a predictor of response to PARPi treatment in prostate cancer based on the probability of treatment discontinuation, according to some embodiments.

FIG. 17B shows the results from a multivariate analysis of predictors of response in prostate cancer, according to some embodiments.

FIG. 18A shows Kaplan-Meier curves demonstrating HRDsig as a predictor of response to PARPi treatment in breast cancer based on the probability of real-world progression-free survival (rwPFS), according to some embodiments.

FIG. 18B shows the results from a multivariate analysis of predictors of response in breast cancer, according to some embodiments.

FIG. 19 shows a consort diagram that describes the eligible patients in the pancreatic CGDB cohort study to test the correlation between patient HRDsig status and responsiveness to platinum-based therapies, e.g. FOLFIRINOX treatment, according to some embodiments.

FIG. 20A shows Kaplan-Meier curves demonstrating the association of HRDsig with first line FOLFIRINOX (FOLF) as a predictor of platinum-containing treatment response in pancreatic cancer based on the probability of real-world overall survival (rwOS), according to some embodiments.

FIG. 20B shows Kaplan-Meier curves demonstrating the association of HRDsig with first line FOLFIRINOX (FOLF) as a predictor of platinum-containing treatment response in pancreatic cancer based on time to next treatment (TTNT), according to some embodiments.

FIG. 20C shows a multivariate analysis of predictors of platinum response in pancreatic cancer. FOLF, FOLFIRINOX (leucovorin, fluorouracil, irinotecan, oxaliplatin); GP, gemcitabine, albumin-bound paclitaxel.

FIG. 21A shows Kaplan-Meier curves demonstrating the association of HRDsig with first line carboplatin+Paclitaxel or docetaxel treatment response in ovarian cancer based on the probability of real-world overall survival (rwOS), according to some embodiments.

FIG. 21B shows Kaplan-Meier curves demonstrating the association of HRDsig with first line platinum-containing treatment response in ovarian cancer based on real-world progression free survival (rwPFS), according to some embodiments.

FIG. 21C shows a multivariate analysis of predictors of platinum response in ovarian cancer for rwOS (top panel) and rwPFS (bottom panel), according to some embodiments.

FIG. 22A shows Kaplan-Meier curves demonstrating the association of HRDsig with first line platinum-containing treatment response in NSCLC cancer based on exploratory real-world progression free survival (rwPFS), according to some embodiments.

FIG. 22B shows a multivariate analysis of predictors of platinum response in ovarian cancer, according to some embodiments.

FIG. 23A shows Kaplan-Meier curves demonstrating the association of HRDsig with first line platinum-containing treatment response in endometrial cancer based on exploratory time to next treatment (TTNT), according to some embodiments.

FIG. 23B shows a multivariate analysis of predictors of platinum response in endometrial cancer, according to some embodiments.

### DETAILED DESCRIPTION OF THE INVENTION

Described herein are computer-implemented methods of identifying a subset of a plurality of features (e.g., genomic data features) using one or more feature importance metrics for training a homologous recombination deficiency (HRD) model (e.g., a classification model). The model is configured to receive test sample data related to the subset of the plurality of features associated with a genome of a tumor in a subject and identify (e.g., classify) the tumor as likely HRD positive or likely HRD negative. Further described herein are methods of identifying (e.g., classifying) a tumor, such as a prostate cancer, ovarian cancer, breast cancer, colorectal cancer, NSCLC, or pancreatic cancer tumor, as likely HRD positive (HRD(+)) or likely HRD negative (HRD(−)). Further described herein are methods of treating a cancer, such as, but not limited to, pancreatic, prostate, ovarian, breast cancer, non-small cell lung cancer (NSCLC), or colorectal cancer (CRC), based on the identification of a tumor as HRD positive (or likely HRD positive) or HRD negative (or likely HRD negative).

Selecting a subset of features can reduce overfitting of the model. Overfitting is problematic because it reduces the scalability of the model and can result in inaccurate classifications (e.g., inaccurate HRD status) because the model ignores scenarios that fall outside of the data used to train the model. Further, by selecting a subset of features that have higher feature importance, the classification model can be trained with less training data and would require less input data. This not only allows for a more efficient modeling process, but also a more accurate classification from a broader range of samples from the model. Further, a model with a reduced set of input features can require less processing power for training and for performing the classification task. Thus, the feature selection process improves the functioning of a computer system by improving processing speed and allowing for efficient use of computer memory and processing power. In addition, by selecting from certain derived copy number features and/or short variant features, the trained model provides greater efficiency and accuracy (e.g., less false-positives/false-negatives) when identifying tumors as HRD-positive or HRD-negative in comparison with previous methods. Previous methods of assessing HRD, such as loss of heterozygosity, telomeric allelic imbalance, and large-scale transition, are subject to noise and error compared with the assessment of derived copy number features and/or short variant features described herein. Proper identification of tumors is integral to being able to appropriately select a treatment for the patient (subject).

Oncogenesis is driven, in part, by the accumulation of somatic alterations of the genomes of cells. Among these alterations include copy number alterations, which are common in many cancers. Loss-of-function, gain-of-function, or gene regulation mutations in certain genes involved in the homologous repair deficiency pathway can lead to accumulation of these copy number alterations. However, other than mutations in certain key genes, such as BRCA1 and BRCA2, the precise combinations of mutations leading to HRD-positive status are unknown. Some tumors will be HRD positive through non-genomic means, for example, through promoter methylation of HRD-associated genes such as BRCA1. Instead of sequencing HRD-associated genes, an alternative approach is to identify and assess the consequences of HRD, such as changes in certain copy number features or in loss of heterozygosity features. However, while both HRD positive and HRD negative genomes may exhibit copy number alterations, the precise values and combinations of features that indicate the presence of HRD are unknown.

Thus, in one aspect, the methods of the invention relate to selecting a subset of features (from a larger plurality of potential features) that can be used to train and operate an HRD classifier process. In another aspect, the methods of the invention relate generally to means of identifying (e.g., classifying) tumors as likely HRD positive (HRD(+)) or likely HRD negative (HRD(−)) based, at least in part, on assessments of features, such as features corresponding to copy number aberrations. This classification is generally based on an assessment of the likelihood that the tumor is HRD-positive or HRD-negative. Based on this assessment, the HRD classifier process may further call the tumor as HRD positive or HRD negative. This classification and/or call may be used as a diagnostic value for the patient having the tumor.

Existing methods for classifying tumors as likely HRD positive or likely HRD negative are often unreliable or imprecise, particularly for HRD positive tumors having wild-type BRCA1 and BRCA2 (which are sometimes described as tumors having a “BRCAness” profile, i.e., those tumors which exhibit similarities to BRCA 1/2-mutant tumors without having the associated BRCA1/2 mutations). Alternatively, not all mutations, even pathogenic mutations such as BRCA1/2 alterations, result in HRD (e.g., some mutations may be monoallelic passengers). Homologous repair deficiency associated with cancer scars the tumor cell genome leading to detectable changes in copy number (i.e., copy number aberrations) and/or indel patterns. The particular pattern, distribution, and form of these copy number aberrations and/or indel patterns can be used to classify tumors into HRD phenotype classes. The present application, in various embodiments, provides means to select the features associated with these patterns (i.e., copy number features) and indel patterns (i.e., short variant features) among other potential features (such as basic features as otherwise described herein) which can be used to identify HRD-positive tumors.

The present application further provides specifically configured models that are based on one or more data features (such as one or more copy number features and/or one or more short variant features) associated with a genome of a cancerous tumor in a subject which can more reliably identify (e.g., classify) said tumors as likely HRD positive or likely HRD negative and optionally call the tumors as HRD positive or HRD negative. The identification (e.g., classification) of a tumor of a cancer in a subject indicates how the tumor should be treated. A trained HRD model using test data comprising at least one or more copy number features, including, for example, one or more of a segment size feature, a sequencing reads feature, an absolute copy number feature, a breakpoint count per x megabases feature, a change point copy number feature, a segment copy number feature, a breakpoint count per chromosome arm feature, a number of segments with oscillating copy number feature, and a segment minor allele frequency feature can be used to identify (e.g., classify) a test tumor as likely HRD positive or likely HRD negative, and also call the tumor as HRD positive or HRD negative based on the likelihood score. These categories of copy number features have been identified as being useful for this identification. Certain categories of short variant features have also been identified as being useful for this identification, including, but not limited to, a deletions (e.g., of at least 5-basepairs) in, for example, microhomology or repetitive regions feature and/or a mutational signature incorporating two or more short variant features.

In combination with one or more of these copy number features and/or one or more of these short variant features, other features or measures may be useful in the described methods, including, but not limited to, certain basic features such as age of subject, cancer type, cancer stage, tumor purity, tumor genome ploidy, and/or tumor genome loss of heterozygosity.

Once a tumor of a cancer in a subject has been identified (e.g., classified) as likely HRD positive or likely HRD negative, or called as HRD positive or HRD negative, it may be treated with an appropriate therapy. For example, if the tumor is identified as likely HRD positive, it may be treated with a drug effective in a HRD positive cancer, such as a platinum-based drug or a PARP inhibitor.

### Definitions

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

The terms “cancer” and “cancerous” refer to or describe the physiological condition in mammals that is typically characterized by unregulated cell growth. Included in this definition are benign and malignant cancers. By “early stage cancer” or “early stage tumor” is meant a cancer that is not invasive or metastatic or is classified as a Stage 0, 1, or 2 cancer. Examples of a cancer include, but are not limited to, a lung cancer (e.g., a non-small cell lung cancer (NSCLC)), a kidney cancer (e.g., a kidney urothelial carcinoma), a bladder cancer (e.g., a bladder urothelial (transitional cell) carcinoma), a breast cancer, a colorectal cancer (e.g., a colon adenocarcinoma), an ovarian cancer, a pancreatic cancer, a gastric carcinoma, an esophageal cancer, a mesothelioma, a melanoma (e.g., a skin melanoma), a head and neck cancer (e.g., a head and neck squamous cell carcinoma (HNSCC)), a thyroid cancer, a sarcoma (e.g., a soft-tissue sarcoma, a fibrosarcoma, a myxosarcoma, a liposarcoma, an osteogenic sarcoma, an osteosarcoma, a chondrosarcoma, an angiosarcoma, an endotheliosarcoma, a lymphangiosarcoma, a lymphangioendotheliosarcoma, a leiomyosarcoma, or a rhabdomyosarcoma), a prostate cancer, a glioblastoma, a cervical cancer, a thymic carcinoma, a leukemia (e.g., an acute lymphocytic leukemia (ALL), an acute myelocytic leukemia (AML), a chronic myelocytic leukemia (CML), a chronic eosinophilic leukemia, or a chronic lymphocytic leukemia (CLL)), a lymphoma (e.g., a Hodgkin lymphoma or a non-Hodgkin lymphoma (NHL)), a myeloma (e.g., a multiple myeloma (MM)), a mycoses fungoides, a merkel cell cancer, a hematologic malignancy, a cancer of hematological tissues, a B cell cancer, a bronchus cancer, a stomach cancer, a brain or central nervous system cancer, a peripheral nervous system cancer, a uterine or endometrial cancer, a cancer of the oral cavity or pharynx, a liver cancer, a testicular cancer, a biliary tract cancer, a small bowel or appendix cancer, a salivary gland cancer, an adrenal gland cancer, an adenocarcinoma, an inflammatory myofibroblastic tumor, a gastrointestinal stromal tumor (GIST), a colon cancer, a myelodysplastic syndrome (MDS), a myeloproliferative disorder (MPD), a polycythemia Vera, a chordoma, a synovioma, an Ewing's tumor, a squamous cell carcinoma, a basal cell carcinoma, an adenocarcinoma, a sweat gland carcinoma, a sebaceous gland carcinoma, a papillary carcinoma, a papillary adenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilms' tumor, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a meningioma, a neuroblastoma, a retinoblastoma, a follicular lymphoma, a diffuse large B-cell lymphoma, a mantle cell lymphoma, a hepatocellular carcinoma, a thyroid cancer, a fallopian tube cancer, a small cell cancer, an essential thrombocythemia, an agnogenic myeloid metaplasia, a hypereosinophilic syndrome, a systemic mastocytosis, a familiar hypereosinophilia, a neuroendocrine cancer, or a carcinoid tumor. In some embodiments, the cancer is a metastatic cancer.

The tumor “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

The terms “individual,” “patient,” and “subject” are used synonymously, and refer to a mammal, and includes, but is not limited to, human, bovine, horse, feline, canine, rodent, or primate. In one embodiment, the subject is a human.

The terms “effective amount” or “therapeutically effective amount” as used herein refer to an amount of a compound, drug, or composition sufficient to treat a specified disorder, condition or disease, such as ameliorate, palliate, lessen, and/or delay one or more of its symptoms. In reference to a cancer, an effective amount comprises an amount sufficient to cause the number of cancer cells present in a subject to decrease in number and/or size and/or to slow the growth rate of the cancer cells. In some embodiments, an effective amount is an amount sufficient to prevent or delay recurrence of the disease. In the case of cancer, the effective amount of the compound or composition may: (i) reduce the number of cancer cells; (ii) inhibit, retard, slow to some extent and preferably stop cancer cell proliferation; (iii) prevent or delay occurrence and/or recurrence of the cancer; and/or (iv) relieve to some extent one or more of the symptoms associated with the cancer.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. In reference to a cancer, the number of cancer cells present in a subject may decrease in number and/or size and/or the growth rate of the cancer cells may slow. In some embodiments, treatment may prevent or delay recurrence of the disease. In the case of cancer, the treatment may: (i) reduce the number of cancer cells; (ii) inhibit, retard, slow to some extent and preferably stop cancer cell proliferation; (iii) prevent or delay occurrence and/or recurrence of the cancer; and/or (iv) relieve to some extent one or more of the symptoms associated with the cancer. The methods of the invention contemplate any one or more of these aspects of treatment.

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

Feature Selection

Starting with a plurality of features (e.g., genomic data features), including those as described otherwise herein, a subset of the plurality of features may be identified using one or more feature importance metrics. Generally, the feature importance metrics allow for evaluation of individual features to determine which features may be most relevant for assessing HRD. Exemplary feature importance metrics include, but are not limited to, gradient boosting (such as XGBoost, also known as XGB), analysis of variance (ANOVA), Chi-Squared analysis, and random forest. Individual features can be assigned values based on these feature importance metrics, where features are assigned increasing importance based on increasing contribution to the performance of the HRD model (e.g., improving performance of the model in classifying tumors as HRD-positive or HRD-negative). Features of higher importance, such as features above a threshold (such as features above median among the plurality of features) may then be selected for use in training or running the HRD model. Once the subset of features is identified, a HRD model (e.g., a classification model) may be trained using the subset of features. The HRD model may then be used to identify (e.g., classify) a tumor of a subject using test data obtained from the tumor and including at least a portion of the features identified during the feature selection.

By selecting this subset of features that have higher feature importance, the model can be trained with less training data and requires less input data, thus improving memory usage and management. Further, a model with a reduced set of input features requires less processing power for training and for performing the identification (e.g., classification) task. Thus, the feature selection process improves the functioning of a computer system by improving processing speed and allowing for efficient use of computer memory and processing power.

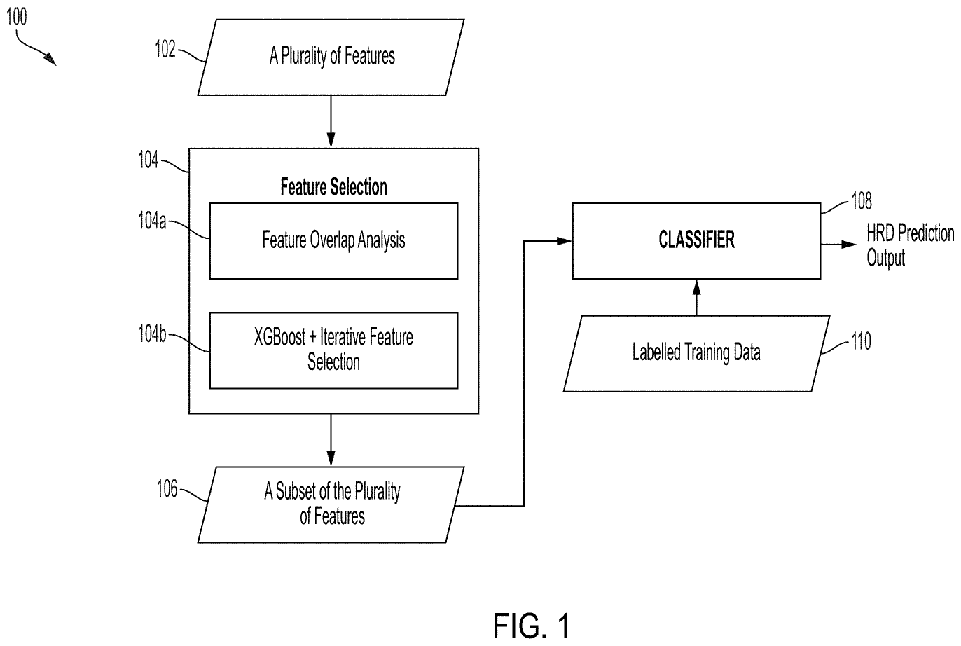

FIG. 1 illustrates an exemplary process for classifying a tumor of a cancer in a subject as HRD-positive or HRD-negative including blocks for identifying a subset of a plurality of features, in accordance with some embodiments. In some embodiments, process 100 is performed, for example, using one or more electronic devices implementing a software platform. In some examples, process 100 is performed using a client-server system, and the blocks of process 100 are divided up in any manner between the server and client device(s). In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At block 102 of FIG. 1, an exemplary system (e.g., one or more electronic devices) receives a plurality of features. In some embodiments, the system receives a dataset comprising a plurality of data elements. A data element can comprise data related to a plurality of features and an associated classification label (e.g. HRD-positive or HRD-negative). For example, a data element can comprise data related to the plurality of features of a sample from a particular subject, and an associated classification label indicating whether the sample is HRD-positive and HRD-negative. The features may include features categorized as basic features, copy number features, and/or short variant features (e.g., a feature corresponding to a base substitution or an indel (insertion or deletion)). Basic features may include, but are not limited to, features related to age of the patient from which the data were obtained, cancer type, cancer stage, tumor purity, tumor genome ploidy, and tumor genome loss of heterozygosity (such as percent of genome under loss of heterozygosity). Copy number features may include, but are not limited to, a segment size feature, a number of sequencing reads feature, an absolute copy number feature, a breakpoint count per x megabases feature, a change point copy number feature, a segment copy number feature, a breakpoint count per chromosome arm feature, a number of segments with oscillating copy number feature, and a segment minor allele frequency feature. Short variant features may include, but are not limited to, a deletions (for example, of at least 5-basepairs) in, for example, homopolymer or repetitive regions feature and/or a mutational signature incorporating two or more short variant features. In some embodiments, one or more of the features are binned features, wherein the values are sorted into bins, such as a binary, a tertile, a quartile, a quintile, a sextile, a septile, or any other suitable binning organization.

At block 104 of FIG. 1, the system and method selects a subset of features from the plurality of features (i.e., the basic features, the copy number features, and/or the short variant features). The subset of features selected may have relatively high predictive value for classifying a tumor of a cancer in a subject as HRD-positive or HRD-negative. In some embodiments, features that have relatively low predictive value and/or are redundant can be excluded from the subset of features in block 104. In some embodiments, the predictive value of a feature may be quantified using a feature importance metric. In some embodiments, the feature importance metric can be applied to obtain a feature importance score for each feature of the plurality of features. The feature importance score of a feature is obtained from a statistical correlation between the feature and the classification label (e.g., HRD-positive or HRD-negative). The statistical correlation between the feature and the classification label may be interpreted based on how much predictive value the feature has for the classification task. In other words, a higher feature importance score can be achieved by having, for example, a higher statistical correlation between the feature and the classification label, which can indicate that the feature plays a more important role in predicting the classification label. By using features that have higher feature importance, a classification model can be trained with less data, thus providing a great degree of efficacy to the training process and less constraints on computer resources (e.g., memory usage, processing speed, etc.). For example, a model with a reduced set of input features can require fewer processing resources to train and perform the classification task. Finally, a model with a reduced set of input features may exhibit less noise and avoid overtraining. Thus, the feature selection process improves the functioning of a computer system by improving the overall efficacy of the training process, improving processing speed, and allowing for efficient use of computer memory and processing resources.

In some embodiments, the system selects the subset of features from the plurality of features received at block 102 of FIG. 1 by performing a feature overlap analysis, as shown by block 104a. At block 104a, each feature importance metric is used to calculate feature importance scores of the plurality of features received from block 102. For each feature importance metric, the system can rank the plurality of features according to their feature importance scores. Thus, the system can obtain a plurality of feature rankings corresponding to the plurality of feature importance features. The system may then identify a subset of features based on the plurality of rankings. The process of ranking the features and identifying the subset of features is described in more detail below.

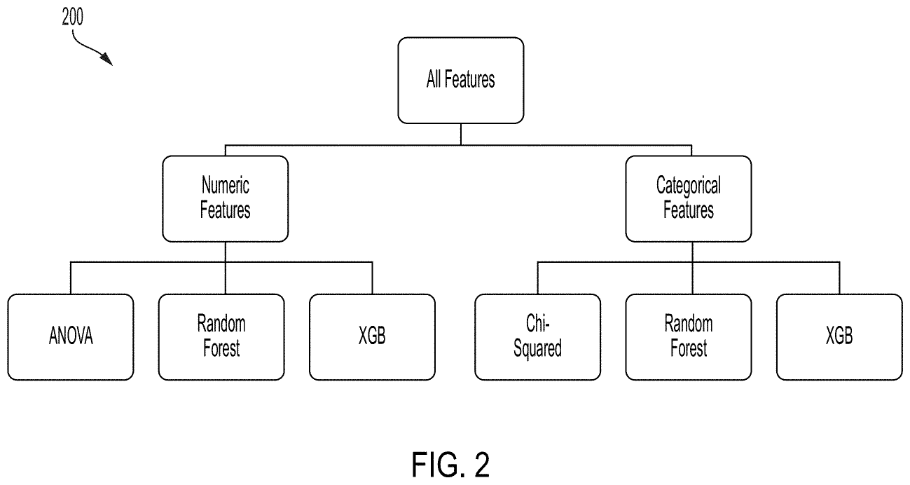

In some embodiments, different types of features can be evaluated using different feature importance metrics. FIG. 2 illustrates a plurality of feature importance metrics that may be used to rank the plurality of features in block 104a in accordance with some embodiments. The depicted exemplary feature importance metrics include ANOVA, random forest, gradient boosting (e.g., XGB), and Chi-Squared. Further, ANOVA can be used to evaluate numeric features of the plurality of features to provide a ranking of the numeric features. Chi-Squared can be used to evaluate categorical features of the plurality of features to provide a ranking of the categorical features. Random forest can be used to evaluate all of the plurality of features to rank all features. Similarly, gradient boosting (such as XGB) can be used to evaluate all of the plurality of features to rank all features.

In some embodiments, the feature importance metrics comprise an analysis of variance (ANOVA) model. ANOVA assesses if there is equal variance between groups (i.e., HRD-positive or HRD-negative) when numeric input variables are compared to a classification target variable. If there is equal variance between groups, then the feature has no impact on the response and it may not be considered for model training. Based on the variance value (f-value), the features may be ranked, and those features that are, for example, above median may be selected as useful features for the model.

In some embodiments, the feature importance metrics comprise a Chi-Square analysis. For feature selection, Chi-Square analysis tests how expected count (i.e., if the feature is independent of output) and observed count deviate from each other. A higher Chi-Square value for a feature indicates it is more dependent on the response variables and is thus more important. Using Chi-Square analysis, features may be ranked, and those features that are, for example, above median may be selected as useful features for the model.

In some embodiments, the feature importance metrics comprise a random forest analysis. During feature selection, for each tree, the prediction accuracy on the out-of-bag portion of the data is recorded. The process is repeated after permuting each predictor variable. The difference between the two accuracies is then averaged over all trees, and normalized by the standard error.

In some embodiments, the feature importance metrics comprise a gradient boosting analysis (e.g., an extreme gradient boosting (XGB) analysis). Gradient boosting, such as XGB, tests the gain contribution of each feature to the model. For a boosted tree model, each gain of each feature of each tree is accounted for, and then the average per feature contribution is assessed. The highest percentage contributor features may then be selected.

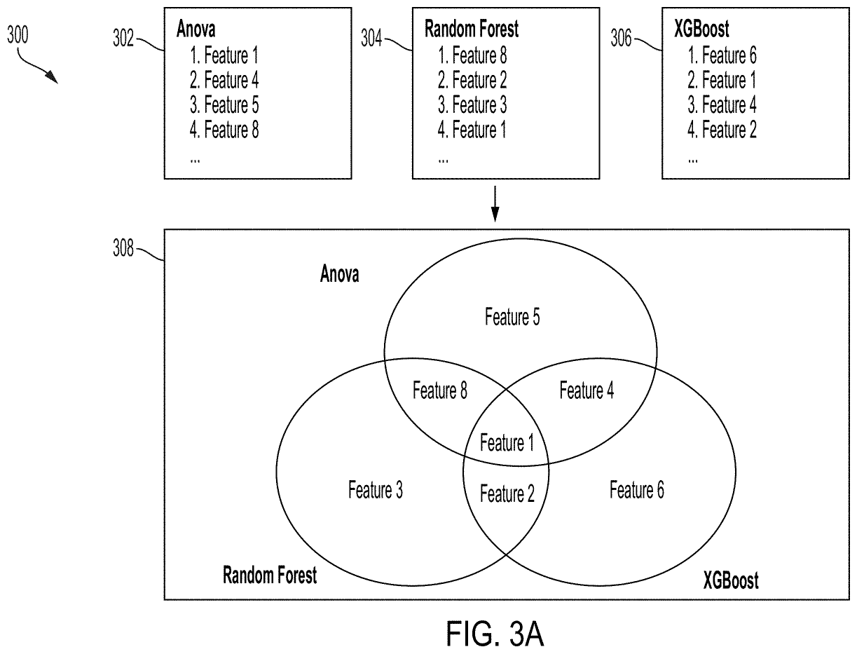

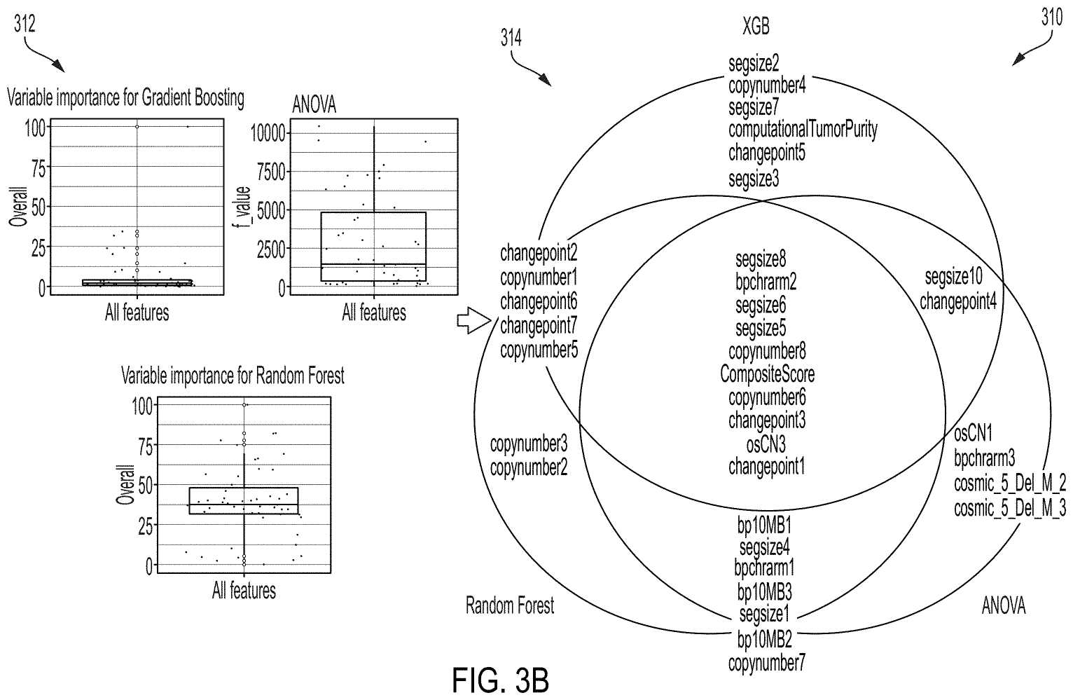

At block 104a of FIG. 1, after the plurality of features are ranked according to feature importance metrics, the system uses the plurality of rankings to select a subset of features. An exemplary process of selecting a subset of features is described in further detail below in FIGS. 3A and 3B.

FIG. 3A illustrates an exemplary feature overlap analysis in accordance with some embodiments. As described above in FIG. 2, a plurality of feature importance metrics may be used to rank a plurality of features. In the example of FIG. 3A, the exemplary process uses an ANOVA, a random forest, and a gradient boosting analysis to rank the features. However, those skilled in the art will understand that other learning techniques known in the art could be used as well. However, for exemplary purposes in FIG. 3A, the ANOVA feature ranking 302 includes features 1, 4, 5, and 8 as the highest ranking features; the random forest ranking 304 includes features 8, 2, 3, and 1 as the highest ranking features; the gradient boosting ranking 306 includes features 6, 1, 4, and 2 as the highest ranking features. In some embodiments, other feature importance metrics may be used to evaluate the features. In some embodiments, fewer or more than three metrics may be used to evaluate the features. In some embodiments, more than four features may be considered as high-ranking features, such as any of more than five, more than six, more than seven, more than eight, more than nine, more than ten, more than eleven, more than twelve, more than thirteen, more than fourteen, more than fifteen, more than sixteen, more than seventeen, more than eighteen, more than nineteen, more than twenty, more than twenty-one, more than twenty-two, more than twenty-three, more than twenty-four, or more than twenty-five features may be considered as high-ranking features.

Once the features have been ranked, the system may perform the feature overlap analysis to determine features that one or more metrics have identified as high-ranking features. In the example of FIG. 3A, feature overlap analysis 308 identifies feature 1 as a high-ranking feature identified in ANOVA feature ranking 302, random forest ranking 304, and gradient boosting ranking 306. Feature overlap analysis 308 also identifies features 2, 4, and 8 as high-ranking features identified by two metrics. In some embodiments, feature overlap analysis 308 may output a subset of features by outputting the features that have been identified as high-ranking by all metrics. In some embodiments, feature overlap analysis 308 may output a subset of features by outputting features that have been identified as high-ranking by one or more metrics. In some embodiments, feature overlap analysis 308 may be graphically represented. In some embodiments, feature overlap analysis 308 may output a list comprising a subset of features.

FIG. 3B illustrates an exemplary output 310 of the feature selection process for features used to classify a tumor of a cancer in a subject as HRD-positive or HRD-negative in accordance with some embodiments. Feature importance rankings 312 are shown graphically, and each graph depicts the ranking of features according to a specific feature importance metric. In each graph (ANOVA, random forest, and gradient boosting), each dot represents a feature, with its y-axis value corresponding to its feature importance as calculated by the feature importance metric. In the example of FIG. 3B, feature overlap analysis 314 may include the top-ranked features according to each feature importance metric. As shown, the feature overlap analysis can identify the features that are highly ranked by all of the metrics and/or some of the metrics.

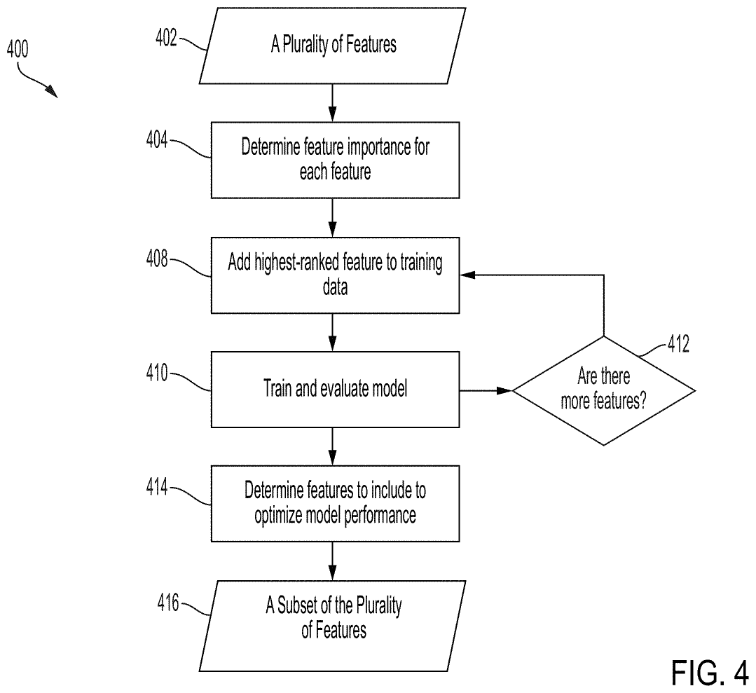

Returning to FIG. 1, in some embodiments, the system and method may determine a subset of a plurality of features using an iterative feature selection process 104b in addition to or as an alternative to process 104a. At block 104b, the system evaluates the features using one or more feature importance metrics (e.g., gradient boosting) and then performs an iterative feature selection process to gradually expand a feature set, as described below in FIG. 4.

FIG. 4 illustrates an iterative feature selection process that may be used by block 104b of FIG. 1 in accordance with some embodiments. At block 402, the system receives a dataset with a plurality of features (e.g., the plurality of features received at block 102 of FIG. 1).

At block 404 of FIG. 4, the system evaluates the features received at block 402 using one or more feature importance metrics (e.g., gradient boosting). The system may then rank the features according to their corresponding feature importance metric scores.

At block 408 of FIG. 4, the system and method obtains a new feature set. In the initial iteration, the system can obtain a new feature set by including the highest-ranking feature(s) as determined by block 404 to the feature set. In a subsequent iteration, the system can expand the existing feature set by adding the next highest-ranking feature(s) as determined by block 404 to obtain a new feature set. The system further obtains a training dataset based with the new feature set. The training dataset can comprise a plurality of data elements, and each data element comprises data related to the new feature set and the corresponding classification label (e.g., HRD-positive or HRD-negative). For example, a data element can comprise data related to the features in the new feature set from a sample and the corresponding classification label (e.g., HRD-positive or HRD-negative) of the sample.

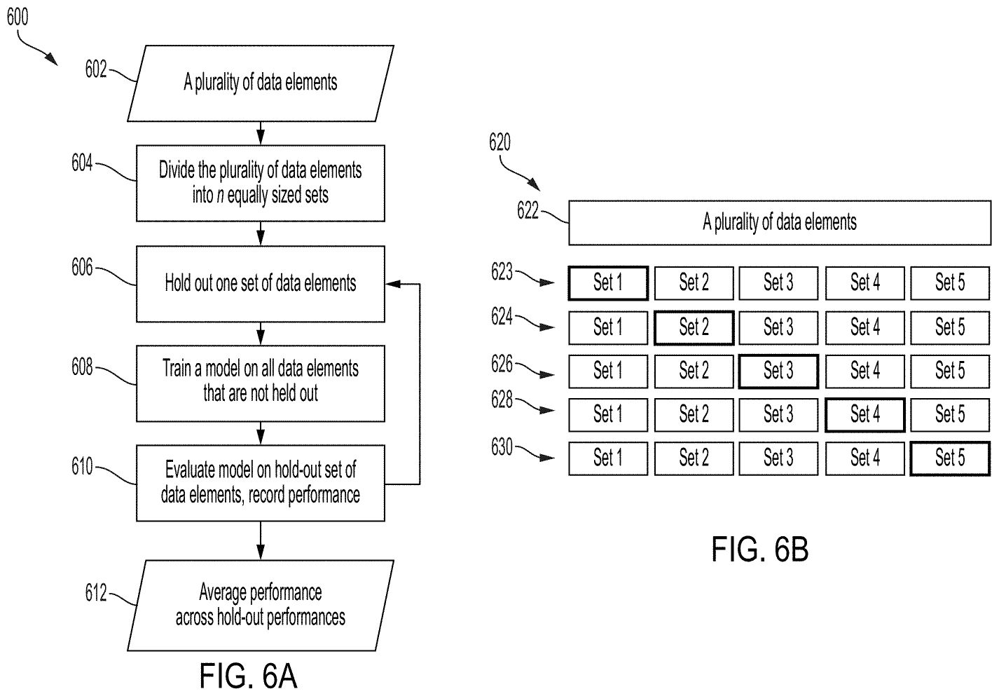

At block 410 of FIG. 4, the system and method trains and evaluates a new classification model using the training dataset from block 408. The system records the model performance in association with the list of features used in the model's training and evaluation. In some embodiments, the training and evaluation of the classification model may be performed using cross-validation methods, as discussed further below by FIGS. 6A and 6B. In some embodiments, the training and evaluation of the classification model may use separate subsets of the dataset from block 408.

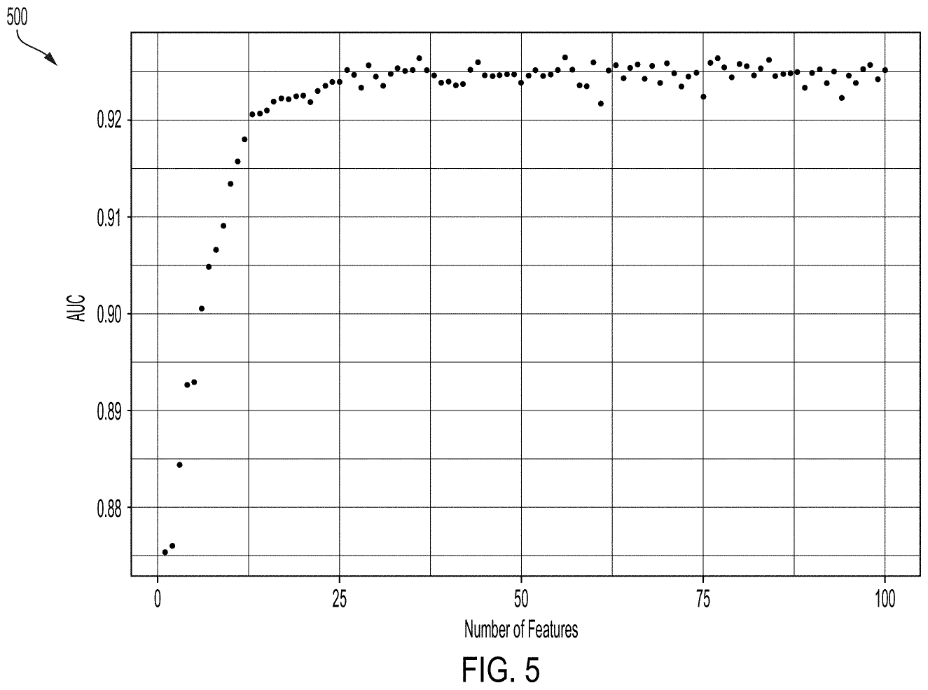

In some embodiments, blocks 408 and 410 of FIG. 4 are iterated until all the features received in block 402 are included in the data. In each iteration, block 408 adds the next highest-ranked feature(s) to the dataset. For example, in the first iteration, block 408 outputs a feature set comprising the highest-ranking feature and a corresponding training set; in the second iteration, block 408 outputs a feature set comprising two highest-ranking features and the corresponding training set; in the third iteration, block 408 outputs a feature set comprising three highest-ranking features and the corresponding training set, and so on. In each iteration, block 410 then trains and evaluates a new classification model using the training dataset from block 406. The system iterates blocks 408 and 410 until a condition is met. In some embodiments, the condition comprises block 412, in which the system determines that there are no more features to be added (e.g. all features received at block 402 are included in the dataset used to train and evaluate the classification model at block 410). In some embodiments, the condition comprises a determination that the performance of the new classification model exceeds a threshold. This iterative process allows the system to record the performance of the classification model when trained and evaluated on the highest-ranking feature, the top two highest-ranking features, the top three highest-ranking features, and so on, until all features received at block 402 are used to train a classification model and evaluated for performance. An example of the recorded performance data is shown below in FIG. 5.

At block 414 of FIG. 4, the system and method utilizes the recorded model performances from block 410 to determine the smallest subset of features that optimizes the performance of the classification model. In some embodiments, the system may determine the smallest subset of features such that adding additional features does not substantially improve model performance. In some embodiments, the system may determine the smallest subset of features such that the classification model performance exceeds a certain predetermined threshold. The subset of features is output at block 414.

FIG. 5 illustrates an example plot of the model performances determined at block 410 of FIG. 4. In the example shown in FIG. 5, the horizontal axis indicates the number of high-ranking features included in the data used to train and evaluate the classification models; the vertical axis indicates the performance of the model. In some embodiments, the performance of the model may be evaluated using area under the receiver operating characteristic (ROC) curve (AUC). In the example of FIG. 5, it may be determined that the 26 highest-ranking features is output as the subset of features in block 416, although a lower number of features may be selected based on the change in the relative increase in model performance with each added feature.

FIG. 6A illustrates an example cross-validation process that may be used to evaluate the performance of a model in accordance with some embodiments. In some embodiments, process 600 may be used at block 410 of FIG. 4 to evaluate the performance of a model. At block 602, the system may receive a plurality of data elements. Each of the plurality of data elements may comprise one or more features and a known classification label. At block 604, the system divides the plurality of data elements from block 602 into n equally-sized subsets. At block 606, the system holds out one of the subsets from block 604 as a “hold-out” set. At block 608, the system trains a model on all data elements that are not held out (e.g. data elements from the n-1 subsets that are not the “hold-out” set). At block 610, the system uses the data elements features from the “hold-out” set as input to the model from block 608. The model generates a plurality of predicted classification labels corresponding to the data elements features. The predicted classification labels are then compared to the known classification labels of the “hold-out” set to evaluate the performance of the model on the “hold-out” set. Blocks 606, 608, and 610 are iterated until all n subsets from block 604 have been used as the “hold-out” set once. That is, blocks 606, 608, and 610 are iterated n times, with a different subset used as the “hold-out” set each iteration. Finally, at step 612, the performances from all n iterations of block 610 are averaged to output an average performance.

FIG. 6B illustrates an example division of the plurality of data elements into five equally sized subsets in accordance with some embodiments. FIG. 6B may be an example of FIG. 6A where n=5. A plurality of data elements 622 may be an example of a plurality of data elements from block 602 of FIG. 6A. In the example of FIG. 6B, plurality of data elements 622 is divided into Set 1, Set 2, Set 3, Set 4, and Set 5. In iteration one 623, at the plurality of data elements 622, Set 1 may be used as the “hold-out” data set as described by block 606. A model may be trained on Set 2, Set 3, Set 4, and Set 5, as described by block 608. The model performance may then be evaluated on “hold-out” data Set 1. This process is then repeated for four more iterations: in iteration two 624, Set 2 is the “hold-out” set, the model is trained on Set 1, Set 3, Set 4, and Set 5, and the model performance is evaluated on Set 2; in iteration three 626, Set 3 is the “hold-out” set, the model is trained on Set 1, Set 2, Set 4, and Set 5, and the model performance is evaluated on Set 3; in iteration four 628, Set 4 is the “hold-out” set, the model is trained on Set 1, Set 2, Set 3, and Set 5, and the model performance is evaluated on Set 4; in iteration five 630, Set 5 is the “hold-out” set, the model is trained on Set 1, Set 2, Set 3, and Set 4, and the model performance is evaluated on Set 5. In the example of FIG. 6B, the average performance may be the average of the model performances from iteration one 622, iteration two 624, iteration three 626, iteration four 628, and iteration five 630.

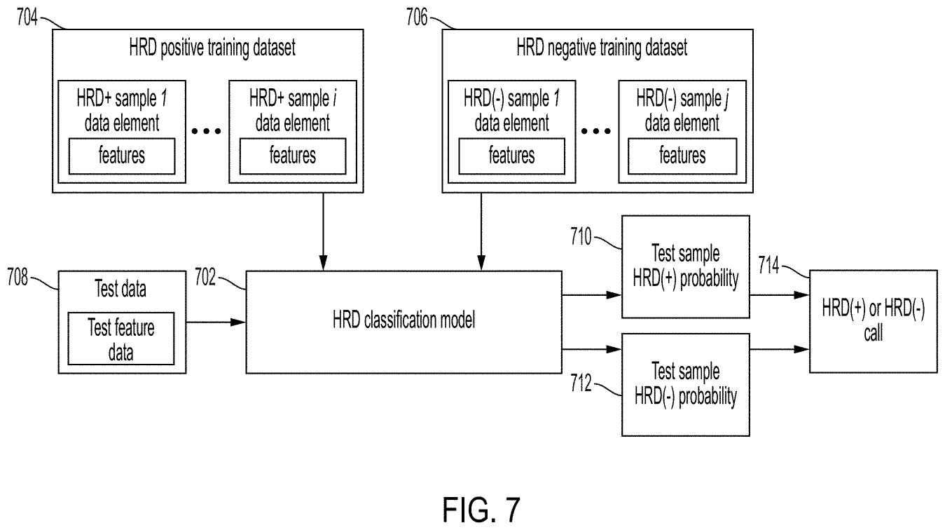

Returning to FIG. 1, at block 106, the system obtains a subset of selected features, as determined by the feature selection of block 104. A classification model 108 is trained using information from selected features 106 and labelled training data 110. In some embodiments, the dataset used for feature selection 104 is the same dataset that is the labelled training data 110. In some embodiments, the dataset used for feature selection 104 is a different dataset from the labelled training data 110. The process of training the classification model is discussed below in the following sections and in FIG. 7. Once classification model 108 is trained, features from an unseen tumor of a cancer in a subject (e.g., data elements that are not included in data received in block 102 and are not associated with known classification labels) could be input into model 108 to predict whether the tumor of a cancer in a subject is likely HRD-positive or HRD-negative.

### Data Features

A test sample from a tumor being identified (e.g., classified) can be obtained from a subject. Features (e.g., genomic features), such as basic features, copy number features, and/or short variant features, associated with the test sample include one or more features that can be used as input for the HRD classification model. The HRD classification model is trained based on corresponding features (such as basic features, copy number features, and/or short variant features) from HRD positive data associated with HRD positive samples (such as tumor samples) and HRD negative data associated with HRD negative samples (such as tumor samples). The features can be used as a functional readout of HRD which can help identify tumors with a “BRCAness” profile, which is associated with HRD. Tumors with such HRD-positive phenotypes may be suitable candidates for certain drug therapies that are not (or often not) effective in HRD-negative phenotypes.

The copy number features can include, but are not limited to, a segment size feature, a number of sequencing reads feature, an absolute copy number feature, a breakpoint count per x megabases feature, a change point copy number feature, a segment copy number feature, a breakpoint count per chromosome arm feature, and a number of segments with oscillating copy number feature. See Macintyre et al., Copy-number signatures and mutational processes in ovarian carcinoma, Nat. Genet. 2018 September; 50(9):1262-1270. Mixture modeling can be applied to divide each feature distribution into mixtures of Gaussian or mixtures of Poisson distributions to achieve float or binary component features. The copy number features can also include a segment minor allele frequency feature, which is based on the A and B allele frequencies of germline SNPs in the segment.

In some embodiments, the HRD model (e.g., the HRD classifier model) may be trained using more features than used as input. For example, the HRD classification model may be trained based on HRD positive data and HRD negative data each comprising a certain number of features associated with the HRD positive tumors and/or HRD negative tumors. The data input to the HRD classification model may then comprise fewer features. The HRD classifier model may, in one example, adjust a weight for data features omitted from the sample data that is input into the trained HRD classifier model. In addition, the HRD classifier model may be trained using additional data features (such as a measure of genome-wide loss of heterozygosity and/or one or more short variant features, each as described herein), but the data input may, in some embodiments, only comprise one or more copy number features associated with the genome of a tumor associated with a cancer in a subject.

To obtain genomic data features, including copy number features, basic features including measures of gLOH and tumor genome ploidy and/or short variant features, sequencing data is collected by sequencing of at least a portion of at least one genome of a tumor. Absolute or relative copy numbers and segmentation can then be derived from whole genome sequencing data, such as shallow whole genome sequencing (sWGS) data. Circular binary segmentation (CBS) may also be used to partition a genome into segments of constant total copy numbers based on DNA microarray data, from which copy number features may be derived. Alternatively, absolute copy numbers and segmentation can be derived from any technique known in the art, including, but not limited to, exome sequencing (ES) or SNP arrays. The distribution of copy number features can be computed from the absolute copy number data, such as the WGS data. Mixture modeling can be applied to divide each feature distribution into mixtures of Gaussian or mixtures of Poisson distributions to achieve float or binary component features. Thus, a particular “copy number feature” used to train the HRD classification model, or to be inputted into the trained HRD classification model, will be expressed as its component feature. For example, for the copy number feature of segment size, if divided into z number of components, then there are z number of possible features which may be used to train the HRD classification model or used to run the HRD classification model. In other words, for a particular test sample, the “copy number feature” in the category of “segment size” (assuming segment size was divided into z number of components) has z number of possible inputs, whether for training or running the HRD classification model. If z is equal to three, then at least one of three segment size features may be input into the HRD classification model: i.e., segsize1, segsize2, or segsize3. Optimal model performance may depend, in part, on the number of component features selected for each particular category of feature. However, particular categories of features may be divided into any suitable number of component features, and not necessarily those corresponding to a particular probability distribution. Thus, the model may perform well and validate efficiently with more or fewer numbers of component features, even if the performance is not optimal.

When deriving copy number features, the absolute copy number data may first be normalized by matching with a normal dataset to determine the baseline level from which to call copy number variation events. The panel of normal is typically derived from healthy tissue samples (which may be from the same individual from which the tumor is derived from). Analysis of the healthy tissue samples allows for setting a baseline copy number from which to derive the copy number features described herein.

Some of the described copy number features may be assessed across subregions of the genome. For example, a particular copy number feature may be assessed across the centromeric portion of the genome. In another example, a copy number feature may be assessed across the telomeric portion of the genome. In yet a further example, a copy number feature may be assessed across both the telomeric and centromeric portions of the genome. In an exemplary method, to define the telomeric and centromeric portions of the genome, a human reference sequence genome, such as hg19, may be used to define the start and end of each chromosome arm. The length of a particular arm is then divided by two to define the halfway point. For each region analyzed for a copy number feature, a segment falling on the centromeric side of this halfway point is defined as a centromeric segment. A segment falling on the telomeric side of this halfway point is defined as a telomeric segment. If a segment spans the halfway point (for example, a segment beginning on the centromeric side and ending on the telomeric side of the halfway point), then that segment may be designated as both centromeric and telomeric, and may be used in the assessment of both telomeric and centromeric copy number features. Any of the data features described herein, as appropriate, may thus be assessed across the telomeric region of the genome, the centromeric region of the genome, or both the telomeric and centromeric regions of the genome.

Modeling of copy number may be impacted by the estimated base ploidy of the genome being assessed. If the base ploidy is estimated higher, floating-point copy number features may be right-shifted, leading to skewed component scores and ultimately incorrect classifications. Normalizing copy number data to the base ploidy involves dividing copy number data by the mean ploidy of the genome being assessed. Thus, any of the described copy number features may be derived from ploidy-normalized copy number data, wherein the absolute copy numbers are normalized to the mean ploidy of the tumor genome. An example method to calculate mean ploidy is to take the weighted average copy number for all segments in a sample. For an exemplary method of calculating mean ploidy, see Sun et al., A computational approach to distinguish somatic vs. germline origin of genomic alterations from deep sequencing of cancer specimens without a matched normal, PLoS Comput. Biol. 2018 Feb. 7; 14 (2):e1005965.

The features described herein may, in some embodiments, be binned features. Feature binning involves organizing certain values to certain categorical bins. For example, for a feature with values ranging from 0 to 10, a quartile binning may organize each of these values from 0 to 10 into one of four bins, wherein lower values may be organized into a lower bin, and higher values into a higher bin. In some embodiments, the binning is unsupervised. In some embodiments, the binning is supervised. In some embodiments, the binning is equal width binning. In equal width binning, the bins have ranges with approximately the same width. For example, for a feature having values from 1 to 8, equal width binning with four bins would organize values of 1 and 2 into a first bin, values of 3 and 4 to a second bin, and so on. In some embodiments, the binning is equal frequency binning. In equal frequency binning, the bins are organized so that each bin has approximately the same number of values, such that the values are distributed about equally into the bins. For example, for a feature having values from 1 to 10, where lower values are much higher frequency, the binning may organize 1 to a first bin, 2 to a second bin, and 3 to 10 in a third bin. The binning may be binary, tertile, quartile, quintile, sextile, septile, or any other suitable binning organization.

In some embodiments of any the described methods, the copy number features comprise a segment size feature. Segment size is derived from the length in genomic bases of each copy number segment across the genome. For example, if a segment has a copy number of x, and the next segment has a copy number of y, then the length of the segment having copy number x and the length of the segment having copy number y are factors in the segment size copy number category. In an exemplary embodiment, the distribution of segment size is divided into 10 component features. A lower-numbered segment size feature represents smaller segment sizes (e.g., segsize1), while a higher-numbered segment size feature represent larger segment sizes (e.g., segsize10). In some embodiments, the distribution of segment size is divided into at least 5 component features, such as at least 6, at least 7, at least 8, at least 9, at least 10, or at least 11 component features. In some embodiments, the distribution of segment size is divided into any of 5, 6, 7, 8, 9, 10, or 11 component features. In some embodiments, the segment size feature is assessed across the telomeric portion of the genome. In some embodiments, the segment size feature is assessed across the centromeric portion of the genome. In some embodiments, the segment size feature is assessed across both the telomeric portion and the centromeric portion of the genome. In some embodiments, the segment size feature is assessed across the entire genome. In some embodiments, the segment size feature is derived from ploidy-normalized copy number data. In some embodiments, the segment size feature is a binned feature.

In some embodiments of any of the described methods, the copy number features comprise a breakpoint count per x megabases feature. In some embodiments, x is between about 1 megabases (MB) and about 150 megabases. In some embodiments, x is any of about 10 MB, about 25 MB, about 50 MB, about 100 MB, and about 150 MB. Breakpoint count per section represents the number of breakpoints per section across the genome or a portion of the genome. For example, for breakpoint count per 10 MB, a processing adjacent window (or, alternatively, a sliding window) of 10 MB is analyzed throughout the genome and the number of breakpoints for each frame of the sliding window can then be assessed. It should be noted that although an adjacent window was used in this approach, a sliding window or any other technique suitable for assessing breakpoints count could be used. Regardless, in some exemplary embodiments, breakpoint count per x megabases is divided into 3 component features. A lower-numbered breakpoint count feature represents fewer breakpoints (e.g., in the case of breakpoint count per 10 MB: bp10MB1, indicating fewer breakpoints per frame of a 10 MB sliding window or per frame of a 10 MB processing adjacent window), while higher-numbered features represent more breakpoints per section (e.g., in the case of breakpoint count per 10 MB: bp10MB3, indicating more breakpoints per frame of a 10 MB sliding window as compared to a lower-numbered feature, such as bp10MB1). In some embodiments, the distribution of breakpoint count is divided into at least 2 component features, such as at least 3 or at least 4 component features. In some embodiments, breakpoint count per section is divided into any of 2, 3, 4, or 5 component features. In some embodiments, the breakpoint count per x megabases feature is assessed across the telomeric portion of the genome. In some embodiments, the breakpoint count per x megabases feature is assessed across the centromeric portion of the genome. In some embodiments, the breakpoint count per x megabases feature is assessed across the entire genome. In some embodiments, the breakpoint count per x megabases feature is derived from ploidy-normalized copy number data. In some embodiments, the breakpoint count per x megabases feature is a binned feature.

In some embodiments of any of the described methods, the copy number features comprise a number of sequencing reads feature obtained from sequencing a genome segment. For a particular genome segment, this value refers to the average number of sequencing reads that align to (i.e., “cover”) the sequenced segment. For genome segments with abnormally high copy number, there will be an increased number of sequencing reads. In contrast, for genome segments that have lost copy number (such as homozygous deletions), there will be fewer sequencing reads. The sequencing reads feature may be expressed as the actual number of reads (such as the average of the reads for each segment analyzed) or a bin of sequencing reads. A lower-numbered sequencing reads feature represents lower absolute sequencing reads, while high-numbered sequencing reads feature represents higher absolute sequencing reads. In some embodiments, sequencing reads feature is assessed across the telomeric portion of the genome. In some embodiments, sequencing reads feature is assessed across the centromeric portion of the genome. In some embodiments, sequencing reads feature is assessed across both the telomeric and centromeric portion of the genome. In some embodiments, sequencing reads feature is derived from ploidy-normalized data. In some embodiments, sequencing reads feature is a binned feature. In some embodiments, the number of sequencing reads feature is a measurement of the number of reads from next generation sequencing (NGS). In some embodiments, the number of sequencing reads feature is expressed as the ratio of sequencing reads for a genome segment in the tumor sample compared to the number of sequencing reads for that genome segment in a control.

In some embodiments of any of the described methods, the copy number features comprise an absolute copy number feature. The absolute copy number may be computed for each genome segment and assigned a value. For example, the assigned values may include 0 (indicating a homozygous deletion), 1 (which may indicate a heterozygous deletion), 2 (which could be a normal count), or more (which may indicate copy number amplification). The absolute copy number feature may represent the actual copy number count (such as the average of the copy number for each segment analyzed) or a bin of copy number values. For example, copy numbers of at least 6 may be binned as representing a high copy number for a segment. Copy numbers between 3 and 5 may be binned as representing a moderately increased copy number. Copy numbers of 1 and 2 may be normal, and copy numbers of 0 may be binned as homozygous deletions. Lower-numbered absolute copy number features represent lower absolute copy number, while high-numbered absolute copy number features represent higher absolute copy number. In some embodiments, absolute copy number is divided into any of 3, 4, 5, 6, 7, 8, or 9 component features. In some embodiments, absolute copy number feature is assessed across the telomeric portion of the genome. In some embodiments, absolute copy number feature is assessed across the centromeric portion of the genome. In some embodiments, absolute copy number features is assessed across both the telomeric and centromeric portions of the genome. In some embodiments, the absolute copy number feature is derived from ploidy-normalized data. In some embodiments, the absolute copy number feature is a binned feature.

In some embodiments of any of the described methods, the copy number features comprise change point copy number feature. Change point copy number refers to the absolute difference in copy number between genome segments across the genome. For example, adjacent segments modeled at copy numbers of 7 and 2 would have an absolute different of 5. In an exemplary embodiment, the distribution of change point copy number is divided into 7 component features. Lower-numbered change point copy number features represent smaller absolute difference in copy number changes (e.g., changepoint1), while higher-numbered features represent larger absolute difference in copy number changes (e.g., changepoint7). In some embodiments, the distribution of change point copy number is divided into at least 4 component features, such as at least 5, at least 6, at least 7, or at least 8 component features. In some embodiments, change point copy number is divided into any of 4, 5, 6, 7, 8, or 9 component features. In some embodiments, the change point copy number feature is assessed across the telomeric portion of the genome. In some embodiments, the change point copy number feature is assessed across centromeric portion of the genome. In some embodiments, the change point copy number feature is assessed across both the telomeric and centromeric portions of the genome. In some embodiments, the change point copy number feature is derived from ploidy-normalized copy number data. In some embodiments, the change point copy number feature is a binned feature.

In some embodiments of any of the described methods, the copy number features comprise a segment copy number feature. Segment copy number is derived from the copy number of each segment across the genome or a portion of the genome. In an exemplary embodiment, the distribution of segment copy number is divided into 8 component features. Lower-numbered segment copy number features represent lower copy numbers (e.g., copynumber1 may represent a copy number level of 0 or 1, or 0 to 1), while higher-numbered copy number features represent higher copy numbers (e.g., copynumber8). In some embodiments, the distribution of segment copy number is divided into at least 4 component features, such as at least 5, at least 6, at least 7, at least 8, or at least 9 component features. In some embodiments, the distribution of segment copy number is divided into any of 4, 5, 6, 7, 8, 9, or 10 component features. In some embodiments, the segment copy number feature is assessed across the telomeric portion of the genome. In some embodiments, the segment copy number feature is assessed across the centromeric portion of the genome. In some embodiments, the segment copy number feature is assessed across the entire genome. In some embodiments, the segment copy number feature is derived from ploidy-normalized copy number data. In some embodiments, the segment copy number feature is a binned feature.

In some embodiments of any of the described methods, the copy number features comprise a breakpoint count per chromosome arm feature. In an exemplary embodiment, the distribution of breakpoint count per chromosome arm is divided into 5 component features. Lower-numbered breakpoint count per chromosome arm features represents fewer breakpoints per arm (e.g., bpchrarm1), while higher-numbered breakpoint count per chromosome arm features represents more breakpoints per chromosome arm (e.g., bpchrarm5). In some embodiments, the distribution of breakpoint count per chromosome arm is divided into at least 3 component features, such as at least 4, at least 5, at least 6, or at least 7 component features. In some embodiments, the distribution of breakpoint count per chromosome arm is divided into any of 4, 5, 6, 7, or 8 component features. In some embodiments, the breakpoint count per chromosome arm is derived from ploidy-normalized copy number data. In some embodiments, the breakpoint count per chromosome arm feature is a binned feature.

In some embodiments, the copy number features comprise a number of segments with oscillating copy number (osCN) feature. Number of segments with oscillating copy number represents a traversal of the genome or a portion of the genome counting the number of repeated alternating segments between two copy numbers. In an exemplary embodiment, the distribution of number of segments with oscillating copy number is divided into 3 component features. Lower-numbered number of segments with oscillating copy number features represents fewer repeated alternations between two copy numbers (e.g., osCN1), while higher-numbered number of segments with oscillating copy number features represents more repeated alternations between two copy numbers (e.g., osCN3). In some embodiments, the distribution of number of segments with oscillating copy number is divided into at least 2, such as at least 3 or at least 4 component features. In some embodiments, the distribution of number of segments with oscillating copy number is divided into any of 2, 3, 4, or 5 component features. In some embodiments, the number of segments with oscillating copy number feature is assessed across the telomeric portion of the genome. In some embodiments, the number of segments with oscillating copy number feature is assessed across the centromeric portion of the genome. In some embodiments, the number of segments with oscillating copy number feature is assessed across the entire genome. In some embodiments, the number of segments with oscillating copy number feature is derived from ploidy-normalized copy number data. In some embodiments, the number of segments with oscillating copy number feature is a binned feature.

In some embodiments, the copy number features comprise a segment minor allele frequency (segMAF) feature. The segMAF feature may be derived from either the mean segMAF or the median segMAF of the tumor genome. In a normal genome at a heterozygous allele site, the expected copy number of each allele is 1.0. HRD is associated with the complete loss of an allele (loss of heterozygosity) or an increase in copy number of one allele relative to the other. Thus, segMAF is a traversal of the genome, segment by segment, comparing the ratio of the minor allele to the major allele. Specifically, each heterozygous SNP is analyzed for the A allele and the B allele frequency; the frequency of the minor allele is captured as the minor allele fraction. Balanced loci will have a ratio of about 0.5:0.5 with a minor allele frequency of 0.5. Loss of heterozygosity events will cause an imbalance and skewing of the minor allele frequency to less than about 0.5 for the minor allele fraction. In some embodiments the segMAF feature is assessed across the telomeric portion of the genome. In some embodiments, the segMAF feature is assessed across the centromeric portion of the genome. In some embodiments, the segMAF feature is assessed across the entire genome. In some embodiments, the segment minor allele frequency feature is a binned feature.

The HRD classification model is trained by HRD positive data comprising, for each HRD positive tumor in a plurality of HRD positive tumors, one or more features associated with the HRD positive tumors and a HRD positive label and HRD negative data comprising, for each HRD negative tumor in a plurality of HRD negative training tumors, one or more copy number features associated with the HRD negative tumors and a HRD negative label. The HRD classification model may also be trained based on other features or measures. Accordingly, test data comprising these other features or measures may be inputted into the HRD classification model (including in combination with the one or copy number features). For example, basic features including, for example, a measure of genomic loss of heterozygosity, and/or one or more short variant features, may be used in the HRD classification model (whether to train the HRD classification model or as test data to be inputted to the HRD classification model).

In some embodiments, the basis features comprise an age of the subject from which the tumor was obtained. The patient may be any age, including any of at least 5, at least 10, at least 15, at least 20, at least 25, at least 30, at least 35, at least 40, at least 45, at least 50, at least 55, at least 60, at least 65, at least 70, at least 75, or at least 80 years old. The age feature may be an integer value for the subject. Alternatively, the age feature may be a qualitative feature, such as any of an infant, young, child, young adult, or elderly subject. In some embodiments, the age feature is a binned feature.

In some embodiments, the basic features comprise a cancer type feature. The cancer type feature refers to the tumor origin. The cancer type may include, for example, one of an adrenal, biliary, bone/soft tissue, breast, colon/rectum, esophageal, eye, head and neck, kidney, liver, lung, lymphoid, medulloblastoma, mesothelioma, myeloid, nervous system, neuroendocrine, ovarian, pancreatic, prostate, skin, stomach, testicle, thymus, thyroid, urinary tract, uterine, or vulvar cancer. In some embodiments, the cancer type feature is a binned feature.

In some embodiments, the basic features comprise a cancer stage feature. Staging of cancers is often based on the type of cancer (e.g., pancreatic cancer staging, prostate cancer staging, breast cancer staging, ovarian cancer staging, etc.), although universal staging systems are also known in the art. Any suitable cancer staging system may be used, and may depend, for example, on the location of the tumor, the cell type, the tumor size, the spread and distribution of the tumor, metastasis of the tumor, and the tumor grade. As a data feature, a cancer stage would typically be expressed as ranging from a less severe stage to a higher severity stage. For example, for a cancer stage feature comprising 4 component features, stage1 may indicate an early-stage cancer, while stage4 may indicate a late-stage cancer. In some embodiments, the cancer stage feature is a binned feature.

The HRD positive data and the HRD negative data is typically split into a training dataset, a validation dataset, and/or a testing dataset. During training, the HRD classification model is only provided with the training set. Optionally, the training set may be balanced. Once trained, the model can be validated by performance on the validation set and tuned. The training may be adjusted and repeated in the event the model exhibits over-fitting on the validation set. Once trained, and after optionally validated, the trained model may be evaluated using the testing dataset.

A measure of genomic loss of heterozygosity (gLOH) (e.g., a genome-wide loss of heterozygosity or exome-wide loss of heterozygosity) may be included as a basic feature in some embodiments. The full genome need not be analyzed to determine the genomic loss of heterozygosity, as whole exome sequencing or targeted sequencing across a large enough portion of the genome may be taken as a proxy from genomic loss of heterozygosity. In some embodiments, the gLOH is encoded as a continuous numeric feature. In some embodiments, the gLOH is encoded as a categorical feature, for example, if the gLOH is above or below a predetermined threshold. The predetermined threshold may be set, for example, at about 10% or higher, about 12% or higher, about 14% or higher, or about 16% or higher. The predetermined threshold may be set, for example, at about 16%. The gLOH may be determined, for example, using the methods described in Swisher et al., Rucaparib in relapsed, platinum-sensitive high-grade ovarian carcinoma (ARIEL2 Part1): an international, multicenter, open-label, phase 2 trial, Lancet Oncology, vol. 18, no. 1, pp. 75-87 (2017).

One or more short variant features may be used in the HRD classification model (whether to train the HRD classification model and/or as test data to be inputted to the HRD classification model). These short variant features may include, but are not limited to, one or more of a deletions (such as at least 5-basepair deletion) at, for example, repetitive or microhomology regions feature and/or a mutational signature incorporating two or more short variant features. These short variant features, in an exemplary method, may be identified by comparing the sequencing data corresponding to a tumor sample with a consensus human genome sequence (such as hg19). In some embodiments, the short variant feature is a binned feature.

Multiple short variant features may be combined and expressed as a mutational signature score. For example, the one or more short variant features may comprise a mutational profile, such as one from the COSMIC cancer database. In one example, the one or more short variant features comprise an indel-based signature, such as the COSMIC ID6 or COSMIC ID8 indel signature of the COSMIC cancer database. Sample profiles can be mapped to these COSMIC profiles, for example, using NNMF methodology. In another example, the one or more short variant features comprise the COSMIC ID8 of the COSMIC cancer database. In yet another example, the one or more short variant features comprise the SBS3 mutational signature of the COSMIC cancer database. For a summary of exemplary COSMIC ID signatures, see Alexandrov et al., The repertoire of mutational signatures in human cancer, Nature 2020; 578(7793):94-101. See also Forbes et al., COSMIC: mining complete cancer genomes in the Catalogue of Somatic Mutations in Cancer, Nuc. Acids Res. 2011 January; 39:D945-D950.

In some embodiments, the one or more short variant features comprise a deletion in microhomology or repetitive regions feature. In some embodiments, the deletions are at least 1-basepair. In some embodiments, the deletions are at least 5-basepairs. Deletions at microhomology regions are a characteristic result of microhomology-mediated end joining (MMEJ), which occurs in the absence of homologous recombination. In this process, short regions of similarity (microhomologies) are used to guide the repair of double stranded breaks in the genome. The identifying characteristic of these deletions is that the 3′ end of the deleted sequence will share similarity with the upstream context of the deletion. Thus, the deletions at a microhomology region feature is a measure of the number of deletions that exhibit this behavior and may also be based on the length of the microhomology (i.e., numerous deletions with longer length vs fewer deletions with shorter lengths).

In an exemplary embodiment, the test data comprise a segment minor allele frequency feature and a segment size feature. In some embodiments, the segment minor allele frequency feature is a binned feature. In some embodiments, the segment size feature is a binned feature. The test data may further comprise at least one of a breakpoint count per x megabases feature, a change point copy number feature, a number of sequencing reads feature, an absolute copy number feature, a segment copy number feature, a breakpoint count per chromosome arm feature, and a number of segment with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a segment minor allele frequency feature and a breakpoint count per x megabases feature. In some embodiments, the segment minor allele frequency feature is a binned feature. In some embodiments, the breakpoint count per x megabases feature is a binned feature. The test data may further comprise at least one of a segment size feature, a number of sequencing reads feature, an absolute copy number feature, a change point copy number feature, a segment copy number feature, a breakpoint count per chromosome arm feature, and a number of segments with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a segment minor allele frequency feature and a change point copy number feature. In some embodiments, the segment minor allele frequency feature is a binned feature. In some embodiments, the change point copy number feature is a binned feature. The test data may further comprise at least one of a segment size feature, a number of sequencing reads feature, an absolute copy number feature, a breakpoint count per x megabases feature, a segment copy number feature, a breakpoint count per chromosome arm feature, and a number of segments with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a segment minor allele frequency feature and a segment copy number feature. In some embodiments, the segment minor allele frequency feature is a binned feature. In some embodiments, the segment copy number feature is a binned feature. The test data may further comprise at least one of a segment size feature, a number of sequencing reads feature, an absolute copy number feature, a breakpoint count per x megabases feature, a change point copy number feature, a breakpoint count per chromosome arm feature, and a number of segments with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a segment minor allele frequency feature and a breakpoint count per chromosome arm feature. In some embodiments, the segment minor allele frequency feature is a binned feature. In some embodiments, the breakpoint count per chromosome arm feature is a binned feature. The test data may further comprise at least one of a segment size feature, a number of sequencing reads feature, an absolute copy number feature, a breakpoint count per x megabases feature, a change point copy number feature, a segment copy number feature, and a number of segments with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a segment minor allele frequency feature and a number of segments with oscillating copy number feature. In some embodiments, the segment minor allele frequency feature is a binned feature. In some embodiments, the number of segments with oscillating copy number feature is a binned feature. The test data may further comprise at least one of a segment size feature, a number of sequencing reads feature, an absolute copy number feature, a breakpoint count per x megabases feature, a change point copy number feature, a segment copy number feature, and a breakpoint count per chromosome arm feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a segment size feature and a breakpoint count per x megabases feature. In some embodiments, the segment size feature is a binned feature. In some embodiments, the breakpoint count per x megabases feature is a binned feature. The test data may further comprise at least one of a segment minor allele frequency (segMAF) feature, a number of sequencing reads feature, an absolute copy number feature, a change point copy number feature, a segment copy number feature, a breakpoint count per chromosome arm feature, and a number of segments with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a segment size feature and a change point copy number feature. In some embodiments, the segment size feature is a binned feature. In some embodiments, the change point copy number feature is a binned feature. The test data may further comprise at least one of a segment minor allele frequency (segMAF) feature, a number of sequencing reads feature, an absolute copy number feature, a breakpoint count per x megabases feature, a segment copy number feature, a breakpoint count per chromosome arm feature, and a number of segments with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a segment size feature and a segment copy number feature. In some embodiments, the segment size feature is a binned feature. In some embodiments, the segment copy number is a binned feature. The test data may further comprise at least one of a segment minor allele frequency (segMAF) feature, a number of sequencing reads feature, an absolute copy number feature, a breakpoint count per x megabases feature, a change point copy number feature, a breakpoint count per chromosome arm feature, and a number of segments with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a segment size feature and a breakpoint count per chromosome arm feature. In some embodiments, the segment size feature is a binned feature. In some embodiments, the breakpoint count per chromosome arm feature is a binned feature. The test data may further comprise at least one of a segment minor allele frequency (segMAF) feature, a number of sequencing reads feature, an absolute copy number feature, a breakpoint count per x megabases feature, a change point copy number feature, a segment copy number feature, and a number of segments with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a segment size feature and a number of segments with oscillating copy number feature. In some embodiments, the segment size feature is a binned feature. In some embodiments, the number of segments with oscillating copy number feature is a binned feature. The test data may further comprise at least one of a segment minor allele frequency (segMAF) feature, a number of sequencing reads feature, an absolute copy number feature, a breakpoint count per x megabases feature, a change point copy number feature, a segment copy number feature, and a breakpoint count per chromosome arm feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a breakpoint count per x megabases feature and a change point copy number feature. In some embodiments, the breakpoint count per x megabases feature is a binned feature. In some embodiments, the change point copy number feature is a binned feature. The test data may further comprise at least one of a segment minor allele frequency (segMAF) feature, a number of sequencing reads feature, an absolute copy number feature, a segment size feature, a segment copy number feature, a breakpoint count per chromosome arm feature, and a number of segments with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a breakpoint count per x megabases feature and a segment copy number feature. In some embodiments, the breakpoint count per x megabases feature is a binned feature. In some embodiments, the segment copy number feature is a binned feature. The test data may further comprise at least one of a segment minor allele frequency (segMAF) feature, a number of sequencing reads feature, an absolute copy number feature, a segment size feature, a change point copy number feature, a breakpoint count per chromosome arm feature, and a number of segments with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a breakpoint count per x megabases feature and a breakpoint count per chromosome arm feature. In some embodiments, the breakpoint count per x megabases feature is a binned feature. In some embodiments, the breakpoint count per chromosome arm feature is a binned feature. The test data may further comprise at least one of a segment minor allele frequency (segMAF) feature, a number of sequencing reads feature, an absolute copy number feature, a segment size feature, a change point copy number feature, a segment copy number feature, and a number of segments with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a breakpoint count per x megabases feature and a number of segments with oscillating copy number feature. In some embodiments, the breakpoint count per x megabases feature is a binned feature. In some embodiments, the number of segments with oscillating copy number feature is a binned feature. The test data may further comprise at least one of a segment minor allele frequency (segMAF) feature, a number of sequencing reads feature, an absolute copy number feature, a segment size feature, a change point copy number feature, a segment copy number feature, and a breakpoint count per chromosome arm feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a change point copy number feature and a segment copy number feature. In some embodiments, the change point copy number feature is a binned feature. In some embodiments, the segment copy number feature is a binned feature. The test data may further comprise at least one of a segment minor allele frequency (segMAF) feature, a number of sequencing reads feature, an absolute copy number feature, a segment size feature, a breakpoint count per x megabases feature, a breakpoint count per chromosome arm feature, and a number of segments with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a change point copy number feature and a breakpoint count per chromosome arm feature. In some embodiments, the change point number feature is a binned feature. In some embodiments, the breakpoint count per chromosome arm feature is a binned feature. The test data may further comprise at least one of a segment minor allele frequency (segMAF) feature, a number of sequencing reads feature, an absolute copy number feature, a segment size feature, a breakpoint count per x megabases feature, a segment copy number feature, and a number of segments with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a change point copy number feature and a number of segments with oscillating copy number feature. In some embodiments, the change point copy number feature is a binned feature. In some embodiments, the number of segments with oscillating copy number feature is a binned feature. The test data may further comprise at least one of a segment minor allele frequency (segMAF) feature, a number of sequencing reads feature, an absolute copy number feature, a segment size feature, a breakpoint count per x megabases feature, a segment copy number feature, and a breakpoint count per chromosome arm feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a segment copy number feature and a breakpoint count per chromosome arm feature. In some embodiments, the segment copy number feature is a binned feature. In some embodiments, the breakpoint count per chromosome arm feature is a binned feature. The test data may further comprise at least one of a segment minor allele frequency (segMAF) feature, a number of sequencing reads feature, an absolute copy number feature, a segment size feature, a breakpoint count per x megabases feature, a change point copy number feature, and a number of segments with oscillating copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a segment copy number feature and a number of segments with oscillating copy number feature. In some embodiments, the segment copy number feature is a binned feature. In some embodiments, the number of segments with oscillating copy number feature is a binned feature. The test data may further comprise at least one of a segment minor allele frequency (segMAF) feature, a number of sequencing reads feature, an absolute copy number feature, a segment size feature, a breakpoint count per x megabases feature, a change point copy number feature, and a breakpoint count per chromosome arm feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

In another exemplary embodiment, the test data comprise a breakpoint count per chromosome arm feature and a number of segments with oscillating copy number feature. In some embodiments, the breakpoint count per chromosome arm feature is a binned feature. In some embodiments, the number of segments with oscillating copy number feature is a binned feature. The test data may further comprise at least one of a segment minor allele frequency (segMAF) feature, a number of sequencing reads feature, an absolute copy number feature, a segment size feature, a breakpoint count per x megabases feature, a change point copy number feature, and a segment copy number feature. The test data may further comprise a measure of gLOH and/or one or more short variant features. The test data may further comprise one or more of an age of the subject from which the test data were obtained, a cancer type feature, a cancer stage feature, a tumor purity feature, and a tumor genome ploidy feature.

### HRD Model

A tumor of a cancer in a subject is classified using a trained HRD classification model that is configured to classify the tumor as HRD-positive (or likely HRD positive) or HRD-negative (or likely HRD negative). The HRD classification model is trained using HRD positive data comprising, for each HRD-positive tumor in a plurality of HRD-positive tumors, one or more data features (such as one or more copy number features and/or one or more short variant features, among other possible features) associated with the HRD-positive tumors and a HRD-positive label. The HRD classification model is further trained using HRD negative data comprising, for each HRD-negative tumor in a plurality of HRD-negative tumors, one or more data features (such as one or more copy number features and/or one or more short variant features, among other possible features) associated with the HRD-negative tumors and a HRD-negative label. Test data comprising one or more data features (such as one or more copy number features and/or one or more short variant features, among other possible features) associated with a genome of a tumor in a subject is input into the trained HRD classification model, which then classifies the tumor as HRD-positive (or likely HRD positive) or HRD-negative (or likely HRD negative) based on the test data.

The models described herein can include one or more machine-learning models, one or more non-machine-learning models, or any combination thereof. The machine-learning models described herein include any computer algorithms that improve automatically through experience and by the use of data. The machine-learning models can include supervised models, unsupervised models, semi-supervised models, self-supervised models, etc. Exemplary machine-learning models include, but are not limited to: linear regression, logistic regression, decision tree, SVM, naïve Bayes, neural networks, K-Means, analysis of variance (ANOVA), Chi-Square analysis, random forest, dimensionality reduction algorithms, and gradient boosting algorithms (such as XGB). The non-machine-learning models can include any computer algorithms that do not necessarily require training and retraining.

The HRD classifier may be a probabilistic classifier, such as a gradient boosting model. The probabilistic classifier can be configured to compute a probability that the tumor is HRD positive or HRD negative, such as by outputting a HRD positive likelihood score or a HRD negative likelihood score. Based on the probability or probabilities outputted from the HRD classification model, the tumor can be called as being HRD positive or HRD negative. Optionally, the tumor may be called as ambiguous, for example if neither the probability that the tumor is HRD positive nor that the probability that the tumor is HRD negative is above a predetermined probability threshold. The HRD positive data and the HRD negative data can include the copy number features and/or the short variant features described herein.

The HRD negative data may comprise genomes with wild-type alleles (i.e., alleles not associated with HRD) at certain HRD-associated genes. For example, in some embodiments, the HRD negative data comprises data associated with genomes with wild-type alleles at one or more of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L. In some embodiments, the HRD negative data comprises promoter methylation data of one or more of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L. In some embodiments, the HRD negative data comprises RNA expression data of one or more of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L. In some embodiments, the HRD negative data comprises data associated with genomes associated with tumors that were found to be resistant to platinum-based drugs (e.g., chemotherapy) and/or PARP inhibitors. In some embodiments, the HRD negative data comprises data associated with genomes associated with tumors previously classified as HRD negative. In some embodiments, the HRD negative data is, at least in part, derived from a consensus human genome sequence, or a portion thereof.

The HRD positive data may comprise data associated with genomes with HRD-associated alleles at certain HRD-associated genes. For example, in some embodiments, the HRD positive data comprises data associated with genomes with mutations at one or more of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L, particularly biallelic mutations thereof. In some embodiments, the HRD positive data comprises promoter methylation data of one or more of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L. In some embodiments, the HRD positive data comprises RNA expression data of one or more of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L. In some embodiments, the HRD positive data comprises data associated with genomes associated with tumors that were found to be sensitive to platinum-based drugs and/or PARP inhibitors. In some embodiments, the HRD positive data comprises data associated with genomes associated with tumors previously classified as HRD positive. In some embodiments, the HRD positive data comprises data associated with tumors having biallelic BRCA1 and BRCA2 mutations associated with HRD.

The HRD positive data may be balanced with the HRD negative data. For example, in an unbalanced training dataset, the number of HRD positive training tumors may outnumber the number of HRD negative tumors (or vice versa). Balancing the data ensures the model has a sufficient number of each label to avoid biasing to one label. When balanced, the number of HRD positive tumors or the number of HRD negative tumors are adjusted so that the ratio between them is at a desired level (such as approximately 1:1 or any other desired ratio). Using the balanced dataset, the HRD classifier may be trained and then tested against a test dataset comprising HRD positive tumors and HRD negative tumors.

The tumors used to train the HRD classifier each comprise an HRD positive label or a HRD negative label. Any suitable methodology may be used to computationally label (e.g., apply a metadata tag to) the tumors as HRD positive or HRD negative. An HRD positive label may be assigned by the presence of alterations in one of the HRD-associated genes, such as one of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L, particularly biallelic alterations thereof. Mutations in one or both of BRCA1 and BRCA2 are especially indicative of HRD positivity, especially biallelic BRCA1/BRCA2 mutations. Tumors may also be labeled as HRD positive based on clinical history. For example, if a tumor was sensitive to a PARP inhibitor or a platinum-based drug regimen, then the tumor is more likely to be HRD positive. An HRD negative label may be assigned based on the absence of alterations in one of the HRD-associated genes, such as one of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L. Mutations in HRD-associated genes may be detected by comparison of the gene sequence with a reference genome, such as a consensus human genome sequence such as hg19. Likewise, tumors may also be labeled as HRD negative based on clinical history. For example, if a tumor was resistant to a PARP inhibitor or a platinum-based drug regimen, then the tumor is more likely to be HRD negative. This is especially true if the tumor was treatment naïve prior to treatment with the PARP inhibitor or platinum-based drug regimen, since HRD positive tumors may develop resistance to these drugs after rounds of treatment. Although each tumor may comprise an HRD positive or HRD negative label, this label does not require absolute certainty that a tumor is HRD positive or HRD negative. Instead, given a robust training dataset comprising numerous HRD positive tumors and numerous HRD negative tumors, and by avoiding overfitting of these data as is known in the art, the contributions of false positives and false negatives are averaged out in the model. Further, the use of a larger training dataset, particularly a balanced training dataset and a dataset having well-defined positive and negative labels (such as by using validated consensus genomes for HRD-negative labels; and by using validated biallelic BRCA1/2 mutants or validated, well-characterized BRCAness samples for HRD-positive labels), allows the model to properly assess the nuanced differences between HRD-negative phenotypes and those exhibiting HRD scarring (i.e., HRD-positive phenotypes).

The classification method is a computer-implemented method. This classification may be executed on a specifically configured machine or system that includes program instructions for executing a trained HRD classifier model, which may be stored on a non-transitory computer readable memory of the computer or system. The computer generally includes one or more processors that can access the memory. The one or more processors can receive data (e.g., test data such as one or more copy number features and/or one or more short variant features associated with a genome of a tumor in a subject and, in some embodiments, other features and measures), which may also be stored on the memory. The one or more processors can access the trained HRD classifier model, and can input the test data into the model. The one or more processors and the trained HRD classifier model can then classify the cancer as likely HRD positive or likely HRD negative.

The HRD classifier model may classify the tumor of the cancer as HRD positive or HRD negative. In some embodiments, the HRD classifier model may classify the tumor as likely HRD positive, likely HRD negative, or ambiguous. For example, the HRD classifier model may classify the tumor as ambiguous if it cannot classify the tumor as likely HRD positive or likely HRD negative with sufficiently high confidence or probability. The confidence or probability threshold may be set by the user as desired, given the tolerance for inaccurate classification. In one example, the user may set the HRD-positive likelihood score threshold at 0.8 and the HRD-negative likelihood score threshold at 0.2. If the HRD-positive likelihood score is below 0.8 and/or if the HRD-negative likelihood score is above 0.2, then the HRD model may not classify the tumor as HRD positive, and would either classify the tumor as HRD negative (depending on how low the HRD-positive likelihood score is and how high the HRD-negative likelihood score is) or ambiguous.

In some embodiments, the HRD classifier outputs a likelihood score that the tumor is HRD positive. In some embodiments, the HRD classifier outputs a likelihood score that the tumor is HRD negative. The HRD classifier may be configured to output either or both of an HRD positive likelihood score and an HRD negative likelihood score. The HRD classifier may also be configured to output a ratio of the HRD positive likelihood score to the HRD negative likelihood score and/or a ratio of the HRD negative likelihood score to the HRD positive likelihood score. The likelihood scores may be expressed as a value from 0.0 (indicating a certainty that the tumor is not HRD positive or HRD negative) to 1.0 (indicating a certainty that the tumor is HRD positive or HRD negative). For example, the trained HRD classifier may receive test sample data comprising a plurality of data features associated with a tumor of a cancer in a subject and output an HRD positive likelihood score of 0.8 and an HRD negative likelihood score of 0.15. The HRD classifier may be configured to call the tumor as HRD positive or HRD negative based upon the likelihood score or scores. In the preceding example, based on the HRD positive likelihood score 0.8 and the HRD negative likelihood score of 0.15, the HRD classifier may call the tumor as HRD positive. In some embodiments, the HRD classifier will call the tumor as HRD positive if the HRD positive likelihood score is at least 0.4, such as at least 0.45, at least 0.5, at least 0.55, at least 0.6, at least 0.65, at least 0.70, at least 0.75, at least 0.80, at least 0.85, at least 0.90, at least 0.95, or at least 0.99. In some embodiments, the HRD classifier will call the tumor as HRD positive if the HRD positive likelihood score is at least 0.7. In some embodiments, the HRD classifier will call the tumor as HRD positive if the HRD positive likelihood score is at least 0.8. In some embodiments, the HRD classifier will call the tumor as HRD positive if the HRD positive likelihood score is at least 0.9. In some embodiments, the HRD classifier will call the tumor as HRD negative if the HRD negative likelihood score is at least 0.4, such as at least 0.5, at least 0.6, at least 0.65, at least 0.70, at least 0.75, at least 0.80, at least 0.85, at least 0.90, at least 0.95, or at least 0.99. In some embodiments, the HRD classifier will call the tumor as HRD negative if the HRD negative likelihood score is at least 0.7. In some embodiments, the HRD classifier will call the tumor as HRD negative if the HRD negative likelihood score is at least 0.8. In some embodiments, the HRD classifier will call the tumor as HRD negative if the HRD negative likelihood score is at least 0.9. In some embodiments, the HRD classifier will call the tumor as HRD positive if the HRD negative likelihood score is less than 0.5, such as less than 0.45, less than 0.40, less than 0.35, less than 0.30, less than 0.30, less than 0.25, less than 0.20, less than 0.15, less than 0.10, or less than 0.05. In some embodiments, the HRD classifier will call the tumor as HRD negative if the HRD positive likelihood score is less than 0.5, such as less than 0.45, less than 0.40, less than 0.35, less than 0.30, less than 0.30, less than 0.25, less than 0.20, less than 0.15, less than 0.10, or less than 0.05. In some embodiments, the HRD classifier will call the tumor as HRD positive if the HRD positive likelihood score is above a certain threshold (such as at least 0.80) and the HRD negative likelihood score is below a certain threshold (such as less than 0.25). In some embodiments, the HRD classifier will call the tumor as HRD negative if the HRD negative likelihood score is above a certain threshold (such as at least 0.80) and the HRD positive likelihood score is below a certain threshold (such as less than 0.25). In some embodiments, the HRD classifier will call the tumor as ambiguous if the HRD positive likelihood score is below a certain threshold and the HRD negative likelihood score is below threshold, or if the absolute values of the likelihood scores are within a threshold percent similarity.

A report may be generated that identifies the cancer as likely HRD positive or likely HRD negative (or ambiguous). The report may be, for example, an electronic medical record or a printed report, which can be transmitted to the subject or a healthcare provider (such as a doctor, a nurse, a clinic, etc.) associated with the subject. The report may be used to make healthcare decisions, such as the method or drug by which the tumor of the cancer is treated.

The report may be displayed on an electronic display or customized interface. For example, in some embodiments, the computer-implemented method may automatically generate the report, and may automatically display the generated report on an electronic display or customized interface.

FIG. 7 shows an exemplary method for training and operating the HRD classification model 702 configured to classify a tumor of a cancer in a subject as HRD-positive or HRD-negative. The HRD classification model 702 is trained using a data set comprising an HRD positive training data set 704 and an HRD negative training data set 706. The HRD positive training dataset 704 includes one or more HRD positive sample data elements (i.e., HRD positive sample 1 data through HRD positive sample i). Each HRD positive sample data element is associated with features (e.g., copy number features, basic features, short variant features, etc.) for HRD positive tumors. The HRD positive sample data element may also include other data features, such as a measure of gLOH and/or short variant features (not shown). The features are labeled as being associated with a HRD positive label. Similarly, the HRD negative training dataset 706 includes one or more HRD negative training sample data elements (i.e., HRD(−) sample 1 through HRD(−) sample j). Each HRD negative sample data element is associated with features (e.g., copy number features, basic features, short variant features, etc.) for HRD negative tumors. The HRD negative sample data element may also include other data features, such as a measure of gLOH and/or short variant features (not shown). The HRD negative samples are labeled as being associated with HRD negative label.

In some embodiments, the HRD classification model 702 is a tree-based gradient boosting model (such as XGBoost). In this model, rather than training all of the models in isolation of one another (e.g., by a random forest), the model is trained in succession such that each new model fits the residuals from the previous models. Therefore, the model achieves a strong classifier from many sequentially-connected weaker classifiers. Repeated cross-validation may be used in the training data for estimating the performance of the HRD classification models.

After classification model 702 has been trained on the training dataset, the classification model 702 may be used to classify a tumor of a cancer in a subject as HRD-positive or HRD-negative. To classify a tumor of a cancer in a subject as HRD-positive or HRD-negative, classification model 702 receives test data 708 comprising test feature data associated with the tumor to be classified. The test data 708 includes one or more copy number features and may include one or more basic features, one or more short variant features, etc. The classification model 702 may determine a probability that the tumor is HRD positive 710 and/or a probability that the tumor is HRD negative 712. The probabilities 710 and 712 are optionally inputted into a HRD calling module 714. The HRD calling module 714 can call the cancer as HRD positive or HRD negative. For example, if the probability that the tumor test sample is HRD positive 710 is greater than the probability that the tumor test sample is HRD negative 712, then the tumor test sample can be called as HRD positive. If the probability that the tumor test sample is HRD negative 712 is greater than the probability that the tumor test sample is HRD positive 710, then the tumor test sample can be called as HRD negative. Optionally, if neither of the probabilities 710 and 712 are above a predetermined threshold, the tumor test sample can be called as ambiguous.

The methods described herein may be implemented using one or more computer systems. Such computer systems can include one or more programs configured to execute one or more processors for the computer system to perform such methods. One or more steps of the computer-implemented methods may be performed automatically. The computer system may include one or more computing nodes. For example, a system may include two or more computing nodes (e.g., servers, computers, routers, or other types of electronic devices that include a network interface), which may be connected and configured to communicate and execute the methods over said network on one or more computing nodes of the network.

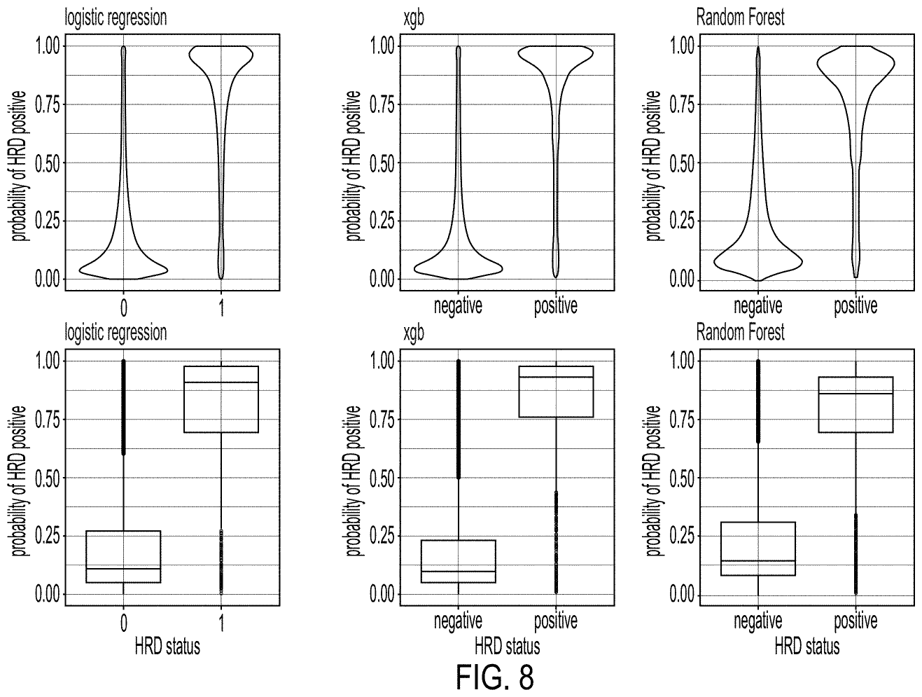

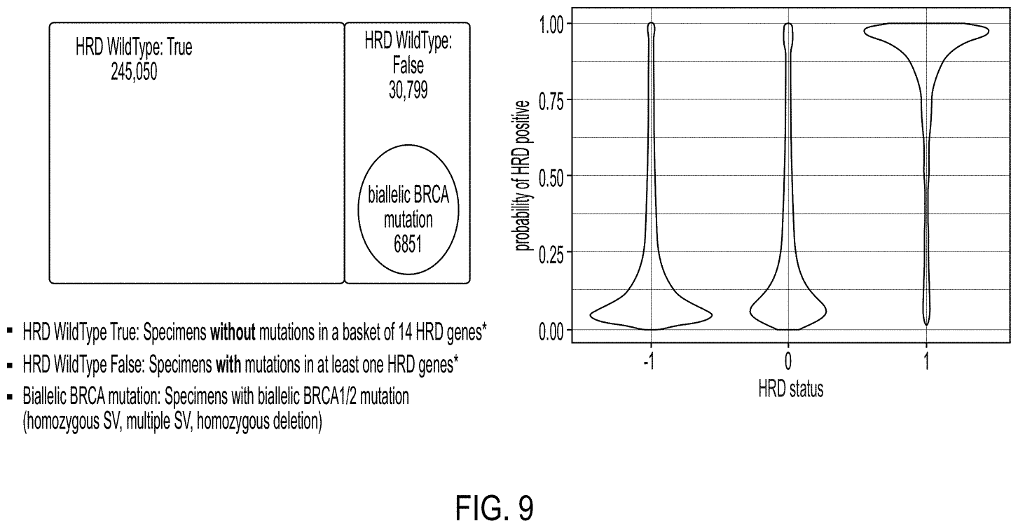

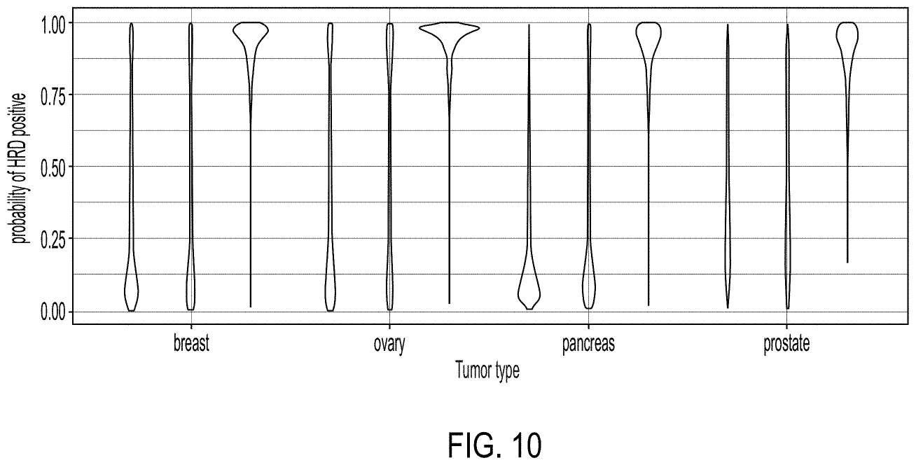

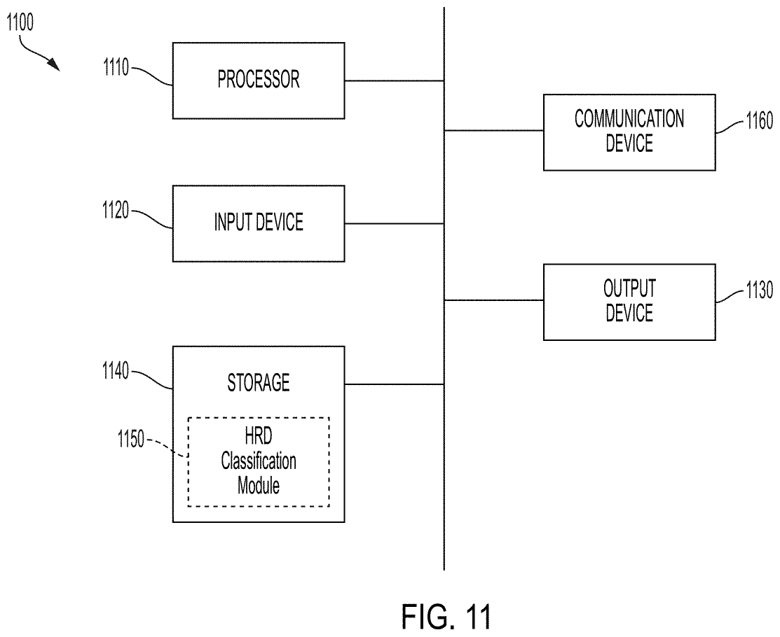

FIG. 8 shows an example of a computing device in accordance with one embodiment. Device 1100 can be a host computer connected to a network. Device 1100 can be a client computer or a server. As shown in FIG. 8, device 1100 can be any suitable type of microprocessor-based device, such as a personal computer, workstation, server or handheld computing device (portable electronic device) such as a phone or tablet. The device can include, for example, one or more of processor 1110, input device 1120, output device 1130, storage 1140, and communication device 1160. Input device 1120 and output device 1130 can generally correspond to those described above, and can either be connectable or integrated with the computer.

Input device 1120 can be any suitable device that provides input, such as a touch screen, keyboard or keypad, mouse, or voice-recognition device. Output device 1130 can be any suitable device that provides output, such as a display, touch screen, haptics device, or speaker.

Storage 1140 can be any suitable device that provides storage, such as an electrical, magnetic or optical memory including RAM, cache, hard drive, or removable storage disk. Communication device 1160 can include any suitable device capable of transmitting and receiving signals over a network, such as a network interface chip or device. The components of the computer can be connected in any suitable manner, such as via a physical bus or wirelessly.

The HRD Classification Module 1150, which can be stored in storage 1140 and executed by processor 1110, can include, for example, one or more program instructions for executing and implementing the methods and process associated with the HRD model (e.g., as embodied in the devices as described above).

The HRD Classification Module 1150 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1140, that can contain or store programming for use by or in connection with an instruction execution system, apparatus, or device.

The HRD Classification Module 1150 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 1100 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1100 can implement any operating system suitable for operating on the network. Software 350 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

### Treatment Methods

Characterization of a tumor as HRD-positive or HRD-negative (or likely HRD-positive or likely HRD-negative) is particularly useful for selecting an effective treatment for a subject having the tumor. Tumors classified as HRD-positive are often more sensitive to certain drugs and therapies that HRD-negative tumors may be resistant to. Based on the classification of a tumor as HRD-positive, likely HRD-positive, HRD-negative, or likely HRD-negative, different drugs or therapies may be selected (e.g., from one or more treatment options). Thus, a method of treating cancer in a subject can include assessing a tumor of the cancer as likely HRD positive or likely HRD negative (or calling a tumor of the cancer as HRD positive or HRD negative), for example according to the methods described herein, and then administering to the subject a therapeutically effective amount of a drug (e.g., a platinum based chemotherapeutic agent or a PARP inhibitor) based on the classification of the tumor as likely HRD positive or likely HRD negative (or based on the call of the tumor as HRD positive or HRD negative). The administration of the drug may be made responsive to the HRD status determination.

The HRD status of the cancer or cancer sample may be used as the basis for a report that identifies one or more treatment options for the subject having cancer. The report may indicate, for example, the subject as one who may benefit, or may not benefit, from a particular treatment, such as a therapy that includes a platinum-based chemotherapeutic agents or a PARP inhibitor. For example, one or more treatment options for a subject having a cancer may be identified (for example, from a plurality of treatment options) by a method that includes determining a homologous recombination deficient (HRD) status of a sample obtained from the subject, and generating a report comprising one or more treatment options identified for the subject based at least in part on the HRD status for the sample, wherein a HRD-positive status in the sample identifies the subject as one who may benefit from treatment with a therapy comprising a platinum-based chemotherapeutic agent or a PARP inhibitor.

The method of treating a cancer in a subject and/or selecting a treatment for a subject having a cancer can include obtaining a classification (i.e., status) of a tumor/cancer, or sample thereof, in the subject as likely HRD positive or likely HRD negative. To obtain this classification, the HRD classification model described herein may be used. One or more copy number features associated with a genome of the tumor of the cancer may be inputted into the HRD classification model which is configured to classify the tumor, based on the one or more copy number features associated with the genome of the tumor in the subject, as likely HRD positive or likely HRD negative. The HRD classification model is trained using HRD positive data from a plurality of HRD positive tumors and HRD negative data from a plurality of HRD negative tumors. The classification may be obtained, for example, by operating the HRD classification model, or by receiving the results from another that operated the HRD classification model.

One or more basic features and/or one or more short variant features may be inputted into the HRD classification model which is configured to classify the tumor based on the one or more basic features and/or the one or more short variant features, as likely HRD positive or likely HRD negative. The one or more short variant features and the one or more basic features may be in addition to, or in the alternative to, the one or more copy number features.

In some embodiments, the treatment or treatment selection methods may include obtaining the test sample data, including the one or more copy number features. In some embodiments, the treatment methods may comprise obtaining the one or more basic features. In some embodiments, the treatment or treatment selection methods may include obtaining the measure of genome-wide loss of heterozygosity. In some embodiments, the treatment or treatment selection methods may include obtaining the one or more short variant features. A test sample may be obtained from the subject, and nucleic acid molecules may be derived from the test sample. The test sample may be, for example, a solid tissue biopsy of the cancer, and nucleic acids may be isolated from the solid tissue sample. Optionally, the test sample may be preserved, for example, by freezing the test sample or fixing the sample (e.g., by forming a formalin-fixed paraffin-embedded (FFPE) sample) prior to isolating the nucleic acid molecules. Alternatively, the test sample is a liquid biopsy sample (e.g., a blood, plasma, or other liquid sample from the subject), and nucleic acids, including circulating tumor DNA (ctDNA), may be obtained from the liquid sample. The nucleic acids from the sample may be assayed and then analyzed to generate any of the one or more copy number features, the one or more basic features, or the one or more short variant features.

Obtaining the classification of the tumor as likely HRD positive or likely HRD negative can include inputting the described features and/or measures into the HRD classification model and classifying, using the features and/or measures, the cancer as likely HRD positive or likely HRD negative based on the data input to the HRD classification model. Alternatively, obtaining the classification of the tumor as likely HRD positive or likely HRD negative may include receiving a report from another entity. The report may be generated by the other entity, and the report can include a classification of the tumor as likely HRD positive or likely HRD negative, wherein the classification is generated using the HRD classification model described herein. In some embodiments, the report includes a likelihood score that the tumor is HRD positive and/or a likelihood score that the tumor is HRD negative, and a final classification can be made based on the likelihood score(s).

Once a classification of the tumor as likely HRD positive or likely HRD negative has been made, a treatment can be selected based on the classification. If the tumor is classified as likely HRD positive, a treatment that is effective in a HRD positive tumor is selected. The selected treatment can then be administered to the subject to treat the tumor that is classified as likely HRD positive. If the tumor is classified as likely HRD negative, a treatment that is not a platinum-based drug or a PARP inhibitor may be selected. The selected treatment can then be administered to the subject to treat the tumor that is classified as likely HRD negative.

Treatments that are effective in a HRD positive tumor can include one or more PARP inhibitors and/or one or more platinum-based agents. PARP inhibitors may include, but are not limited to, veliparib, olaparib, talazoparib, iniparib, rucaparib, and niraparib. PARP inhibitors are described in Murphy and Muggia, PARP inhibitors: clinical development, emerging differences, and the current therapeutic issues, Cancer Drug Resist 2019; 2:665-79. Platinum-based agents may include, but are not limited to, cisplatin, oxaliplatin, and carboplatin. Platinum-based drugs are described in Rottenberg et al., The rediscovery of platinum-based cancer therapy, Nat. Rev. Cancer 2021 January; 21(1):37-50.

The tumor to be treated is a tumor in a subject. In one embodiment, the tumor is a pancreatic cancer. In another embodiment, the tumor is a prostate cancer. In some embodiments, the tumor is an ovarian, breast, or prostate cancer. In some embodiments, the tumor is a tumor associated with HRD, which may include, but is not limited to, one of adrenal, biliary, bone/soft tissue, breast, colon/rectum, esophageal, eye, head and neck, kidney, liver, lung, lymphoid, medulloblastoma, mesothelioma, myeloid, nervous system, neuroendocrine, ovarian, pancreatic, prostate, skin, stomach, testicle, thymus, thyroid, urinary tract, uterine, or vulvar cancer. See Nguyen al., Pan-cancer landscape of homologous recombination deficiency, Nat. Commun. 2020 Nov. 4; 11(1):5584.

In some embodiments, the therapy may further include one or more of leucovorin (folinic acid), fluorouracil, or both. In some embodiments, the therapy may further include a topoisomerase inhibitor. Exemplary topoisomerase inhibitors include, but are not limited to, an anthracycline (e.g., doxorubicin, epirubicin, idarubicin, daunorubicin, etc.), etoposide, ciprofloxacin, and irinotecan. In some embodiments, the therapy includes irinotecan.

In some embodiments, the therapy is FOLFIRINOX, which is a chemotherapy combination that includes a platinum-based chemotherapeutic agent (namely, oxaliplatin), along with folinic acid (leucovorin), fluorouracil, and irinotecan. FOLFIRINOX is generally administered according to known treatment regimens, for example, 85 mg/m² oxaliplatin over 2 hours, followed by 400 mg/m² folinic acid over 2 hours in combination with 180 mg/m² irinotecan over 90 minutes started 30 minutes after initiating folinic acid administration, followed by 400 mg/m² fluorouracil bolus, followed by 2400 mg/m² fluorouracil administered over about 46 hours, although variations of this treatment regimen may be administered (for example by increasing or decreasing doses of certain drug components of the combination). See, for example, Conroy et al., FOLFIRINOX versus Gemcitabine for Metastatic Pancreatic Cancer, New England J. of Med., vol. 364, pp. 1817-1825 (2011); Jung et al., Planned and actual dose reduction of standard or modified FOLFIRINOX in metastatic pancreatic cancer: A systematic review and meta-analysis, J. Clinical Oncology, vol. 40, no. 16 supp., e16279 (2022). FOLFIRINOX may be administered in a plurality of therapy cycles, for example every two weeks. In an example, there is a method of treating a homologous recombination deficient (HRD)-positive cancer in a subject, comprising: identifying the cancer as an HRD-positive cancer, comprising: obtaining genomic data comprising values for a plurality of genomic features for the cancer; inputting, by one or more processors, the genomic data into a trained HRD model configured to characterize the cancer as HRD-positive or HRD-negative based on the genomic data; and characterizing, by the one or more processors, using the trained HRD model, the cancer as HRD-positive; and responsive to identifying the cancer as an HRD-positive cancer, administering oxaliplatin, fluorouracil, irinotecan, an doxaliplatin (FOLFIRINOX) to the subject.

In some embodiments, the therapy may further include an immune-oncology (TO) therapy, which may include the administration of one or more IO agents. In some embodiments, the IO therapy comprises an immune checkpoint inhibitor. In some embodiments, the immune checkpoint inhibitor comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof. In some embodiments, the immuno-oncology (IO) therapy comprises an immune checkpoint inhibitor, for example, an immune checkpoint inhibitor targeting PD-1, PD-L1, CTLA-4, or a combination thereof. In some embodiments, the immune checkpoint inhibitor is a PD-1 inhibitor. In some embodiments, the immune checkpoint inhibitor comprises one or more of nivolumab, pembrolizumab, cemiplimab, or dostarlimab. In some embodiments, the immune checkpoint inhibitor is a PD-L1-inhibitor. In some embodiments, the immune checkpoint inhibitor comprises one or more of atezolizumab, avelumab, or durvalumab. In some embodiments, the immune checkpoint inhibitor is a CTLA-4 inhibitor. In some embodiments, the CTLA-4 inhibitor comprises ipilimumab. In some embodiments, the immune checkpoint inhibitor comprises a nucleic acid. In some embodiments, the nucleic acid comprises a double-stranded RNA (dsRNA), a small interfering RNA (siRNA), or a small hairpin RNA (shRNA). In some embodiments, the immune checkpoint inhibitor comprises a cellular therapy. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In one embodiment, the tumor or cancer is a pancreatic cancer. In another embodiment, the tumor or cancer is a prostate cancer. In some embodiments, the tumor or cancer is an ovarian, breast, or prostate cancer. In some embodiments, the tumor or cancer is a tumor or cancer associated with HRD, which may include, but is not limited to, one of adrenal, biliary, bone/soft tissue, breast, colon/rectum, esophageal, eye, head and neck, kidney, liver, lung, lymphoid, medulloblastoma, mesothelioma, myeloid, nervous system, neuroendocrine, ovarian, pancreatic, prostate, skin, stomach, testicle, thymus, thyroid, urinary tract, uterine, fallopian tube cancer, endometrial cancer, or vulvar cancer. In some embodiments, the cancer is metastatic. In some embodiments, the cancer is breast cancer, ovarian cancer, prostate cancer, pancreatic cancer, lung cancer, non-small cell lung cancer (NSCLC), colorectal cancer (CRC), uterine cancer, fallopian tube cancer, endometrial cancer, or urothelial cancer. In some embodiments, the cancer is breast cancer. In some embodiments, the cancer is ovarian cancer. In some embodiments, the cancer is prostate cancer. In some embodiments, the cancer is pancreatic cancer. In some embodiments, the cancer is lung cancer. In some embodiments, the cancer is non-small cell lung cancer (NSCLC) cancer. In some embodiments, the cancer is colorectal cancer (CRC) cancer. In some embodiments, the cancer is uterine cancer. In some embodiments, the cancer is fallopian tube cancer. In some embodiments, the cancer is endometrial cancer. In some embodiments, the cancer is urothelial cancer.

### Identification, Prognostic, Monitoring, and Other Methods

The HRD status of a cancer (or status of a sample from the subject that indicates the status of the cancer) may be used to identify an individual having the cancer for treatment with a therapy comprising a platinum-base chemotherapeutic agent or a PARP inhibitor. Thus, in some embodiments, a method for identifying an individual having a cancer for treatment with a therapy comprising a platinum-based chemotherapeutic agent or a PARP inhibitor can include determining a homologous recombination deficient (HRD) status of a sample obtained from the subject, and identifying the subject for therapy comprising the platinum-based chemotherapeutic agent or the PARP inhibitor if the HRD status of the sample is identified as HRD-positive. The HRD status of the sample may be determined in accordance with the methods described herein.

The HRD status of a cancer (or status of a sample from the subject that indicates the status of the cancer) may be used to predict survival of the subject having cancer. For example, if the cancer or sample of the HRD cancer is HRD-positive, the subject may be predicted to have longer survival when treated with a therapy comprising a platinum-based chemotherapeutic agent or a PARP inhibitor, as compared to treatment comprising a therapy without the platinum-based chemotherapeutic agent or the PARP inhibitor. Accordingly, in some embodiments, there is a method of predicting survival of a subject having a cancer, comprising acquiring knowledge of a homologous recombination deficient (HRD) status of a sample obtained from the subject, wherein the HRD status for the sample obtained from the subject is a HRD-positive status, and wherein responsive to the acquisition of said knowledge, the subject is predicted to have longer survival when treated with a therapy comprising a platinum-based chemotherapeutic agent or a PARP inhibitor, as compared to treatment comprising a therapy without the platinum-based chemotherapeutic agent or the PARP inhibitor. The HRD status of the sample may be determined in accordance with the methods described herein.

The HRD status of a cancer (or status of a sample from the subject that indicates the status of the cancer) may be used to monitor, evaluate, or screen a subject having cancer. A subject having an HRD-positive status may be predicted to have longer survival when treated with a therapy comprising a platinum-based chemotherapeutic agent or a PARP inhibitor, as compared to treatment comprising a therapy without the platinum-based chemotherapeutic agent or the PARP inhibitor. For example, a method of monitoring, evaluating, or screening a subject having a cancer, comprising acquiring knowledge of a homologous recombination deficient (HRD) status of a sample obtained from the subject, wherein the HRD status for the sample obtained from the subject is a HRD-positive status, and wherein responsive to the acquisition of said knowledge, the subject is predicted to have longer survival when treated with a therapy comprising a platinum-based chemotherapeutic agent or a PARP inhibitor, as compared to treatment with a therapy without the platinum-based chemotherapeutic agent or the PARP inhibitor. The HRD status of the sample may be determined in accordance with the methods described herein.

The HRD status of a cancer (or status of a sample from the subject that indicates the status of the cancer) of a subject may be used to stratify the subject, for example in a clinical trial. If the HRD status is a HRD-positive status, identifying the subject as a candidate for receiving the platinum-based chemotherapeutic agent or the PARP inhibitor. If, on the other hand, the HRD status is a HRD-negative status, identifying the subject as a candidate for receiving treatment without the platinum-based chemotherapeutic agent or the PARP inhibitor. Accordingly, in some embodiments, there is a method of of stratifying a subject with a cancer for a treatment with a therapy comprising a platinum-based chemotherapeutic agent or a PARP inhibitor, comprising acquiring knowledge of a homologous recombination deficient (HRD) status of a sample obtained from the subject, and (a) if the HRD status is a HRD-positive status, identifying the subject as a candidate for receiving the platinum-based chemotherapeutic agent or the PARP inhibitor; or (b) if the HRD status is a HRD-negative status, identifying the subject as a candidate for receiving treatment without the platinum-based chemotherapeutic agent or the PARP inhibitor. The HRD status of the sample may be determined in accordance with the methods described herein.

For any of the above methods a classification (i.e., status) of the cancer (or sample) in the subject as HRD positive or likely HRD negative may be obtained. To obtain this classification, the HRD classification model described herein may be used. One or more copy number features associated with a genome of the tumor of the cancer may be inputted into the HRD classification model which is configured to classify the tumor, based on the one or more copy number features associated with the genome of the tumor in the subject, as likely HRD positive or likely HRD negative. The HRD classification model may be trained using HRD positive data from a plurality of HRD positive tumors and HRD negative data from a plurality of HRD negative tumors. The classification may be obtained, for example, by operating the HRD classification model, or by receiving the results from another that operated the HRD classification model.

One or more basic features and/or one or more short variant features may be inputted into the HRD classification model which is configured to classify the tumor based on the one or more basic features and/or the one or more short variant features, as likely HRD positive or likely HRD negative. The one or more short variant features and the one or more basic features may be in addition to, or in the alternative to, the one or more copy number features.

In some embodiments, the method may include obtaining the test sample data, including the one or more copy number features. In some embodiments, the method may comprise obtaining the one or more basic features. In some embodiments, method may include obtaining the measure of genome-wide loss of heterozygosity. In some embodiments, the method may include obtaining the one or more short variant features. A test sample may be obtained from the subject, and nucleic acid molecules may be derived from the test sample. The test sample may be, for example, a solid tissue biopsy of the cancer, and nucleic acids may be isolated from the solid tissue sample. Optionally, the test sample may be preserved, for example, by freezing the test sample or fixing the sample (e.g., by forming a formalin-fixed paraffin-embedded (FFPE) sample) prior to isolating the nucleic acid molecules. Alternatively, the test sample is a liquid biopsy sample (e.g., a blood, plasma, or other liquid sample from the subject), and nucleic acids, including circulating tumor DNA (ctDNA), may be obtained from the liquid sample. The nucleic acids from the sample may be assayed and then analyzed to generate any of the one or more copy number features, the one or more basic features, or the one or more short variant features.

Obtaining the classification of the tumor as likely HRD positive or likely HRD negative can include inputting the described features and/or measures into the HRD classification model and classifying, using the features and/or measures, the cancer as likely HRD positive or likely HRD negative based on the data input to the HRD classification model. Alternatively, obtaining the classification of the tumor as likely HRD positive or likely HRD negative may include receiving a report from another entity. The report may be generated by the other entity, and the report can include a classification of the tumor as likely HRD positive or likely HRD negative, wherein the classification is generated using the HRD classification model described herein. In some embodiments, the report includes a likelihood score that the tumor is HRD positive and/or a likelihood score that the tumor is HRD negative, and a final classification can be made based on the likelihood score(s).

Treatments that are effective in a HRD positive tumor, which may be selected or identified according to the above methods, can include one or more PARP inhibitors and/or one or more platinum-based agents. PARP inhibitors may include, but are not limited to, veliparib, olaparib, talazoparib, iniparib, rucaparib, and niraparib. Platinum-based agents may include, but are not limited to, cisplatin, oxaliplatin, and carboplatin.

In some embodiments, the therapy may further include one or more of leucovorin (folinic acid), fluorouracil, or both. In some embodiments, the therapy may further include a topoisomerase inhibitor. Exemplary topoisomerase inhibitors include, but are not limited to, an anthracycline (e.g., doxorubicin, epirubicin, idarubicin, daunorubicin, etc.), etoposide, ciprofloxacin, and irinotecan. In some embodiments, the therapy includes irinotecan.

In some embodiments, the therapy is FOLFIRINOX, which is a chemotherapy combination that includes a platinum-based chemotherapeutic agent (namely, oxaliplatin), along with folinic acid (leucovorin), fluorouracil, and irinotecan.

In some embodiments, the therapy may further include an immune-oncology (IO) therapy, which may include the administration of one or more IO agents. In some embodiments, the IO therapy comprises an immune checkpoint inhibitor. In some embodiments, the immune checkpoint inhibitor comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof. In some embodiments, the immuno-oncology (IO) therapy comprises an immune checkpoint inhibitor, for example, an immune checkpoint inhibitor targeting PD-1, PD-L1, CTLA-4, or a combination thereof. In some embodiments, the immune checkpoint inhibitor is a PD-1 inhibitor. In some embodiments, the immune checkpoint inhibitor comprises one or more of nivolumab, pembrolizumab, cemiplimab, or dostarlimab. In some embodiments, the immune checkpoint inhibitor is a PD-L1-inhibitor. In some embodiments, the immune checkpoint inhibitor comprises one or more of atezolizumab, avelumab, or durvalumab. In some embodiments, the immune checkpoint inhibitor is a CTLA-4 inhibitor. In some embodiments, the CTLA-4 inhibitor comprises ipilimumab. In some embodiments, the immune checkpoint inhibitor comprises a nucleic acid. In some embodiments, the nucleic acid comprises a double-stranded RNA (dsRNA), a small interfering RNA (siRNA), or a small hairpin RNA (shRNA). In some embodiments, the immune checkpoint inhibitor comprises a cellular therapy. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In one embodiment, the tumor or cancer is a pancreatic cancer. In another embodiment, the tumor or cancer is a prostate cancer. In some embodiments, the tumor or cancer is an ovarian, breast, or prostate cancer. In some embodiments, the tumor or cancer is a tumor or cancer associated with HRD, which may include, but is not limited to, one of adrenal, biliary, bone/soft tissue, breast, colon/rectum, esophageal, eye, head and neck, kidney, liver, lung, lymphoid, medulloblastoma, mesothelioma, myeloid, nervous system, neuroendocrine, ovarian, pancreatic, prostate, skin, stomach, testicle, thymus, thyroid, urinary tract, uterine, fallopian tube cancer, endometrial cancer, or vulvar cancer. In some embodiments, the cancer is metastatic. In some embodiments, the cancer is breast cancer, ovarian cancer, prostate cancer, pancreatic cancer, lung cancer, non-small cell lung cancer (NSCLC), colorectal cancer (CRC), uterine cancer, fallopian tube cancer, endometrial cancer, or urothelial cancer. In some embodiments, the cancer is breast cancer. In some embodiments, the cancer is ovarian cancer. In some embodiments, the cancer is prostate cancer. In some embodiments, the cancer is pancreatic cancer. In some embodiments, the cancer is lung cancer. In some embodiments, the cancer is non-small cell lung cancer (NSCLC) cancer. In some embodiments, the cancer is colorectal cancer (CRC) cancer. In some embodiments, the cancer is uterine cancer. In some embodiments, the cancer is fallopian tube cancer. In some embodiments, the cancer is endometrial cancer. In some embodiments, the cancer is urothelial cancer.

### Data Generation

Features (e.g., genomic data comprising values for genomic features) may be obtained for the cancer or cancer sample. At least a portion of the features, may be obtained, for example, by sequencing nucleic acids associated with the cancer.

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy)

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenstrom macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, Wis.); and the Maxwell 16 Buccal Swab LEV DNA Purification Kit Technical Manual (Promega Literature #TM333, Jan. 1, 2011, Promega Corporation, Madison, Wis.)). Protocols for RNA isolation are disclosed in, e.g., the Maxwell® 16 Total RNA Purification Kit Technical Bulletin (Promega Literature #TB351, August 2009, Promega Corporation, Madison, Wis.).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, Md.) or the Maxwell® and ReliaPrep™ series of kits from Promega (Madison, Wis.).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, Ga., product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (0 assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S.L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is —1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### EXAMPLES

The following examples are included for illustrative purposes only and are not intended to limit the scope of the invention.

### Example 1— Analysis of HRD Signature Predicts PARP Inhibitor Response in Cancer Patients

PARP inhibitors (PARPi) are approved for multiple indications with ongoing trials to explore broader utility. However, identifying the right patients for these therapies remains a challenge. In ovarian cancer, genomic scar based measures for homologous recombination deficiency (HRD) are approved diagnostics (genome-wide LOH [gLOH] and genomic instability score [GIS]); however, broader utility has not been established.

As described, an algorithm to predict HRD status using indel and copy number features (referred herein as “HRDsig”) was developed. Using this algorithm, across the pan-cancer data set, the rate of HRDsig+ was 6.4% with the highest frequency in fallopian tube (30%), ovarian (30%), peritoneal (23%), breast (16%), and prostate cancers (15%). Sensitivity to detect biallelic BRCA1/2 alterations was high across tumors [ovary (93%), prostate (87%), breast (85%), pancreas (80%)]. Beyond BRCA1/2, HRDsig positivity was associated with biallelic alterations in PALB2 (OR=33, p<1E-10), BARD1 (OR=17, p<1E-10), RAD51C (OR=14, p<1E 10), and RAD51D (OR=13, p<1E-10), and RAD51B (OR=4, p<2E-09).. In an ovarian cancer clinic-genomic database (CGDB), 220 patients were treated with PARPi (HRDsig positive=109, HRDsig negative=111). HRDSig positivity was associated with improved TTD (median 9.0 months versus 4.0 months; HR=0.50[0.36-0.70], p<0.001), with similar predictive power to gLOH>16% (HR=0.49 [0.33-0.72], p=0.001) and GIS>42 (HR=0.69 [0.50-0.97], p=0.03). For 72 patients with prostate cancer treated with PARPi (median 6.8 months versus 3.2 months; HR=0.50 [0.26-0.96], p=0.036). While both gLOH and GIS identified fewer patients than HRDsig (gLOH: 22 v 31; GIS 14 v 31), each trended predictive for PARPi TTD (gLOH —high: HR=0.53 [0.24-1.18], p=0.3); GIS>42: HR=0.44 [0.17-1.09], p=0.08). These findings suggest that HRD is associated with genomic scarring beyond ovarian cancer.

Data on clinical outcomes were collected as part of the nationwide (US-based) de-identified ovarian and metastatic prostate clinico-genomic databases (CGDBs). The de-identified data originated from approximately 280 US cancer clinics (˜800 sites of care). Retrospective longitudinal clinical data were derived from electronic health record (EHR) data, comprising patient-level structured and unstructured data, curated via technology-enabled abstraction, and were linked to genomic data derived from comprehensive genomic profiling (CGP) tests in the CGDB by de-identified, deterministic matching (see, e.g., Singal et al., Association of Patient Characteristics and Tumor Genomics With Clinical Outcomes Among Patients With Non-Small Cell Lung Cancer Using a Clinicogenomic Database, JAMA, vol. 321, no. 14, pp. 1391-1399 (2019)). The study included 4,292 and 3,551 patients diagnosed with ovarian and metastatic prostate cancer, respectively. Clinical characteristics and oral therapy usage were obtained via technology-enabled abstraction of clinical notes and radiology/pathology reports and linked to CGP data. Additional inclusion criteria were applied to select a cohort for outcomes analysis: 1) an ovarian carcinoma diagnosis (ovarian) or mCRPC diagnosis (prostate) date in 2011 or later, 2) a diagnosis date no greater than 90 days following first structured activity, 3) relevant CGP result no more than 60 days following last clinical visit, and 4) tissue CGP with collection date prior to initiation of PARPi. These criteria resulted in 220 and 72 eligible PARPi treated patients with ovarian or prostate cancer, respectively. Treatment outcomes were retrospectively analyzed for patients in the ovarian and prostate cancer clinico-genomic databases. Median time to treatment discontinuation (TTD) was estimated with Kaplan-Meier analysis and adjusted (age at PARPi initiation, line number, race, ECOG and prior platinum exposure (ovarian only)) hazard ratios (HR) from Cox proportional hazards models. PARPi treatment was considered discontinued if a subsequent line of systemic therapy which did not include PARPi was documented, structured data was present 90 days or more following the last documented drug episode date, or if the patient died within 90 days of the most recent drug episode date. All other patients were censored at the last abstracted PARPi date. For prostate patients, therapy line numbers were indexed from the date of castrate resistant disease. Therapies received prior to this date were not counted towards cumulative line count.

Tumor samples were sequenced by hybrid capture-based comprehensive genomic profiling in a CLIA certified/CAP-accredited laboratory as part of routine clinical care. See, Frampton et al., Development and validation of a clinical cancer genomic profiling test based on massively parallel DNA sequencing. Nat. Biotechnol., vol. 31, no. 11, pp. 1023-1031 (2013). Sequencing of liquid biopsy ctDNA was performed on ≥20 ng of ctDNA extracted from blood plasma to create adapter sequencing libraries before hybrid capture and sample-multiplexed sequencing to a median unique exon coverage depth of >6,000× for up to 324 genes (see Woodhouse et. al., Clinical and analytical validation of FoundationOne Liquid CDx, a novel 324-Gene cfDNA-based comprehensive genomic profiling assay for cancers of solid tumor origin, PLoS One, vol. 15, no. 9, e0237802 (2020). Results were analyzed for base substitutions, short insertions and deletions (indels), copy number alterations, and rearrangements. Genome-wide copy-number profiles were generated using >10,000 SNPs and on-target baits throughout the genome.

Copy number features were extracted, and signatures were called using methods similar to that of Macintyre et al, Copy number signatures and mutational processes in ovarian carcinoma, Nat. Genet., vol. 50, no. 9, pp. 262-1270 (2018)). Mixture modeling was performed on TP53 mutated ovarian carcinoma tissue biopsy samples that passed QC metrics (low copy number noise, computational purity >=15%, they did not contain significant contamination, and they were not a confirmed transplant). Values were ploidy-normalized and captured additional breakpoint bins beyond Macintyre (LOMB, 25 MB, 50 MB, and 100 MB).

Fraction focal genome-wide loss of heterozygosity (gLOH) was calculated by quantifying loss of heterozygosity across the genome but excluding whole chromosome arm losses (>90% arm loss), a method that was described and validated in the ARIEL2 and ARIEL3 clinical trials for ovarian cancer. Genome instability scores were calculated from our segmented copy number profiles and represented a sum of qualifying LOH segments, TAI events, and LST adjusted for ploidy by subtracting 15.5*ploidy.

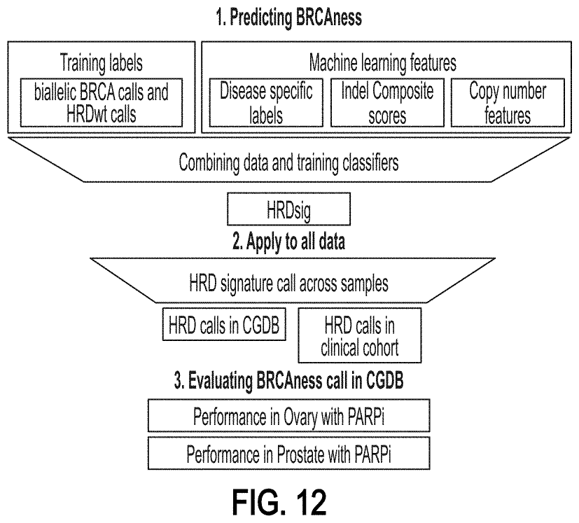

HRDSig Prediction. FIG. 12 provides an overview of a method of training an HRD signature (HRDsig) model to call HRDsig-positive or HRDsig-negative cancers, applying the model to characterize samples with associated clinciopathological data, and evaluating performance of PARPi therapy in ovarian or prostate cancer.

A pan-cancer genomic profiling data set was split 70:30 for training and validation of an HRD signature using an XGB machine learning model. A broad set of copy number features (Macintyre et al, Copy number signatures and mutational processes in ovarian carcinoma, Nat. Genet., vol. 50, no. 9, pp. 262-1270 (2018)) and indel features were used to identify signatures of HRD. gLOH and GIS were calculated using copy number profiles. Biallelic alterations were predicted using a computational zygosity algorithm (see Sun et al., A computational approach to distinguish somatic vs. germline origin of genomic alterations from deep sequencing of cancer specimens without a matched normal, PLOS: Computational Biology, vol. 14., no. 2, e1005965 (2018). The HRR geneset included BRCA1, BRCA2, PALB2, RAD51C, RAD51D, BARD1, ATM, CHEK1, CHEK2, BRIP1, CDK12, FANCL, RAD51B, and RAD54L. This pan-cancer analysis identified the ubiquity of copy number signatures across nearly all tumor types. Copy number and indel features were utilized to train a machine learning based HRD caller (HRDsig), which has predictive value for PARPi benefit in real-world datasets of ovarian, prostate, and breast cancers.

The data was divided into training and testing datasets (70:30). For feature selection, all the features to fit a model in training dataset was initially used. The features were then ranked by their importance based on their performance gain. Copy number features and indel features were included in constructing the extreme gradient boosting (XGB) machine learning model. Biallelic BRCA1/2 and homologous recombination repair wildtype (HRRwt; wildtype for BRCA1, BRCA2, ATM, BARD1, BRIP1, CDK12, CHEK1, CHEK2, FANCL, PALB2, RAD51B, RAD51C, RAD51D, RAD54L alterations) were defined as true positive and true negative training labels.

The optimal number of features was selected until the ROC-AUC plateaued in the training dataset. The optimal model was determined using grid search over the hyper-parameter space with 4 repeated 10-fold cross-validation and the performances are evaluated by ROC-AUC. During development, it was noted that genomic scarring of the prostate is different than other tumor types, so two XGB models were developed: one applied to pan-cancer and the other applied to prostate only. Both XGB models (pan-cancer and prostate-specific) were trained in the same process.

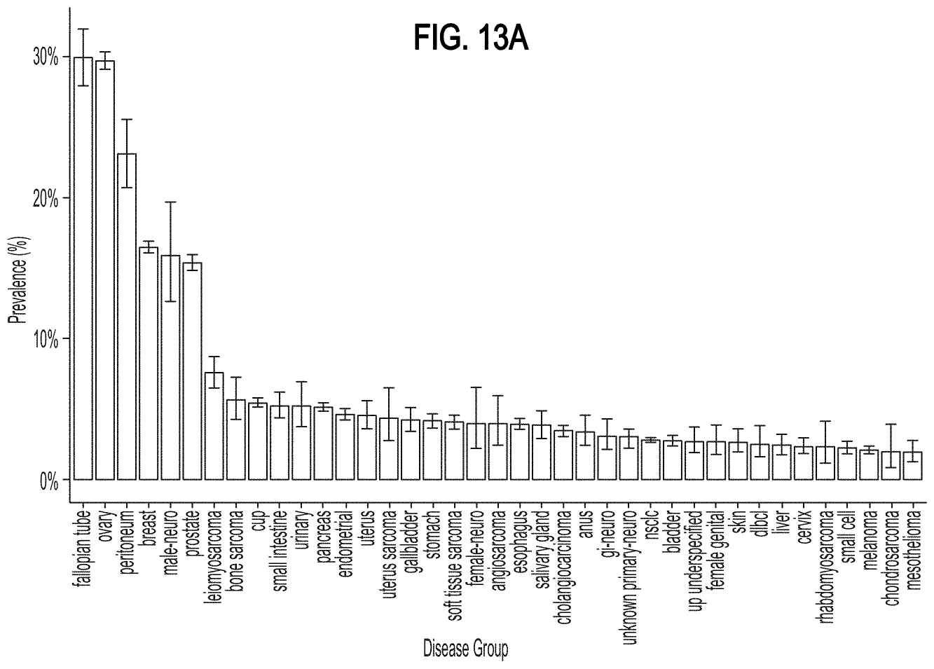

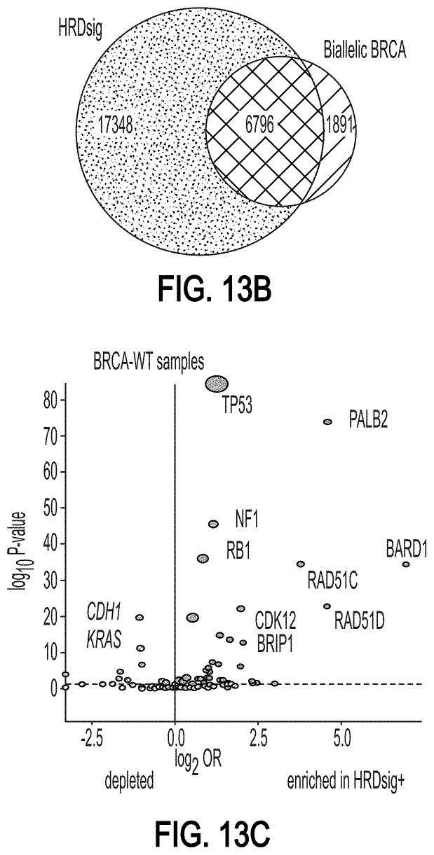

Across the pan-cancer dataset, the prevalence of HRDsig positivity was 6.4% with the highest frequencies in fallopian tube cancers (30%), ovarian cancers (30%), peritoneal cancers (23%), breast cancers (16%), and prostate cancers (15%) (FIG. 13A). Diseases with the lowest prevalence of HRDsig included thyroid cancers (0.5%), acute leukemias (0.2%), and myelodysplastic syndromes (0.1%). FIG. 13B shows the overlap of HRDsig-positive patients with patients harboring bialleleic BRCA1/2 alterations. FIG. 13C shows co-occurrence of biallelic gene alterations with HRDsig in BRCA1/2 wt patients, where HRDsig positivity was strongly associated with biallelic alterations in BARD1 (OR=122, p<1E-10), PALB2 (OR=24, p<1E-10), RAD51D (OR=24, p<1E-10), and RAD51C (OR=14, p<1E-10).

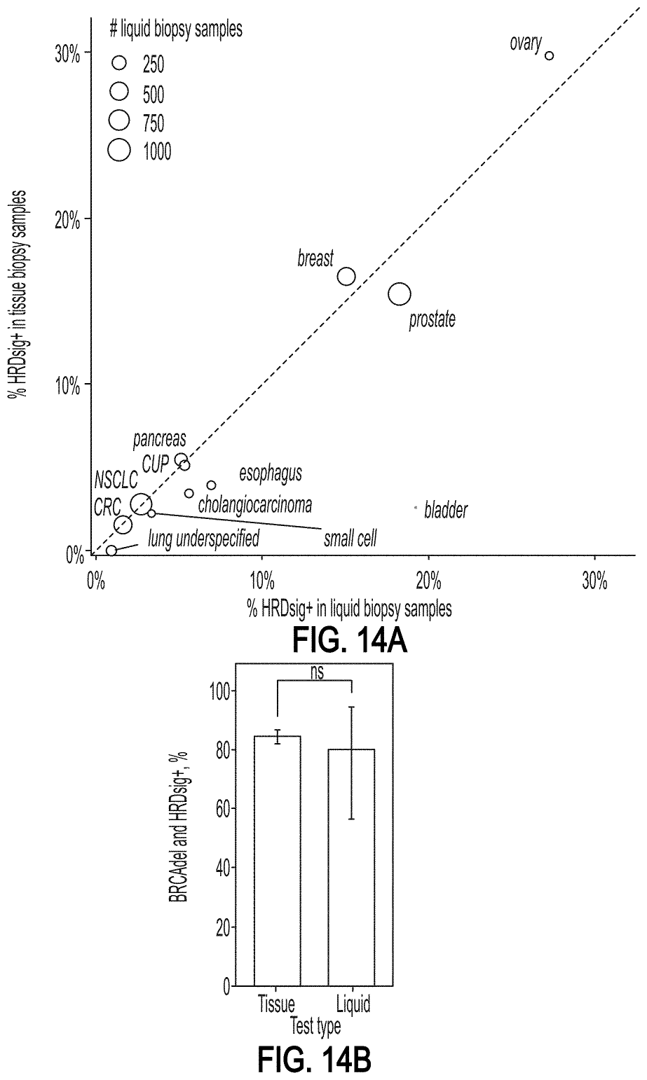

The algorithm was trained using tissue biopsy samples, although liquid biopsies are able to detect indels and copy number events when sufficient circulating tumor DNA (ctDNA) is present. The frequency of HRDsig in high shedding samples (tumor fraction >10%) was examined using an unmodified algorithm. The frequency of HRDsig-positivity was similar in tissue and liquid across diseases (e.g. 30% v 27% for tissue and liquid in ovarian cancer, 15% v 18% in prostate cancer, and 16% v 15% in breast cancer). These findings suggest the HRD signatures may be applicable to liquid biopsy samples when sufficient ctDNA is present. HRDSig calling in liquid biopsies with elevated tumor fraction (TF>10%) was compared to HRDSig calling tissue biopsy samples and liquid biopsy samples. FIG. 14A shows the frequency of HRDsig positivity in tissue biopsy (y-axis) or liquid biopsy (x-axis) across disease groups. FIG. 14B shows frequency of HRDsig positivity in samples with BRCA1/2 deletions in tissue and liquid biopsies. Examination of copy number profiles for breast cancer patients profiled with tissue and liquid biopsies found similar copy number patterns.

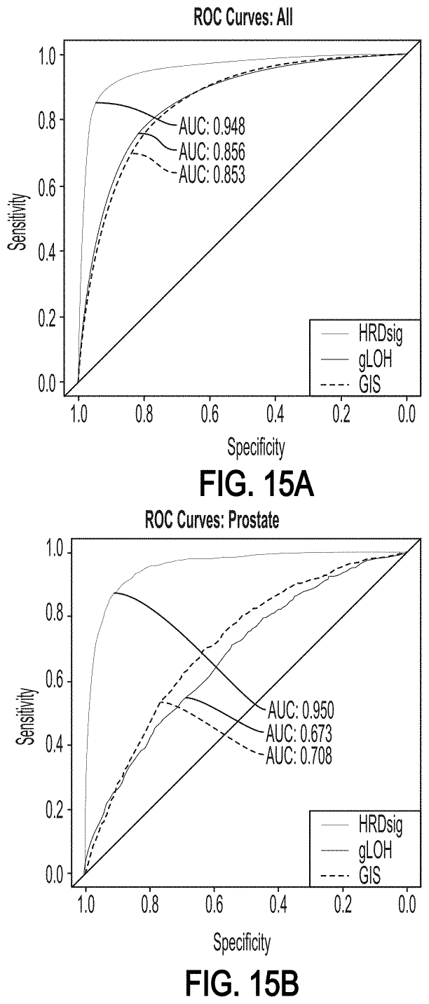

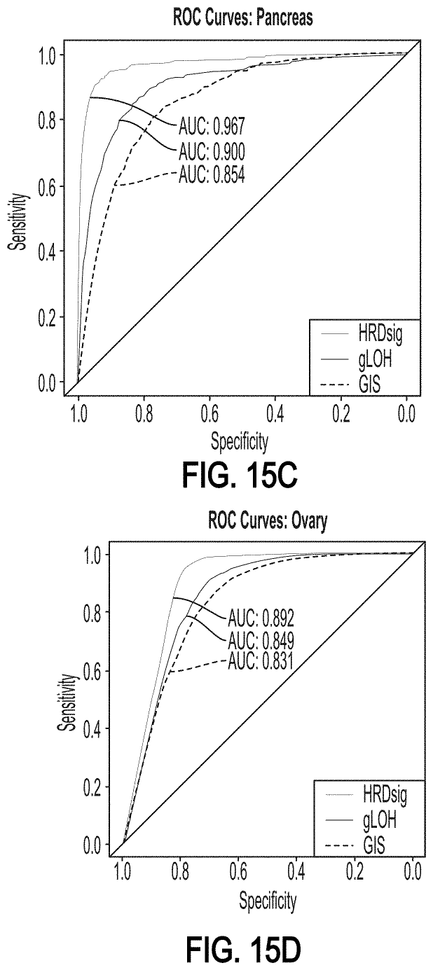

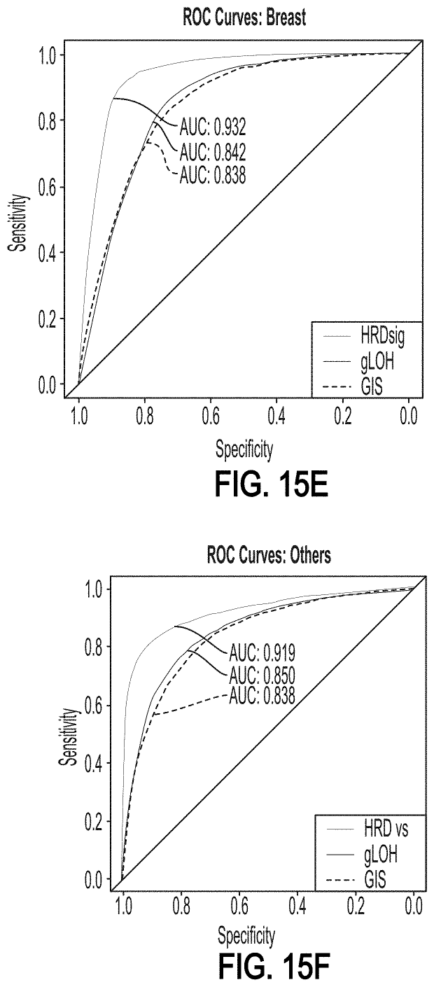

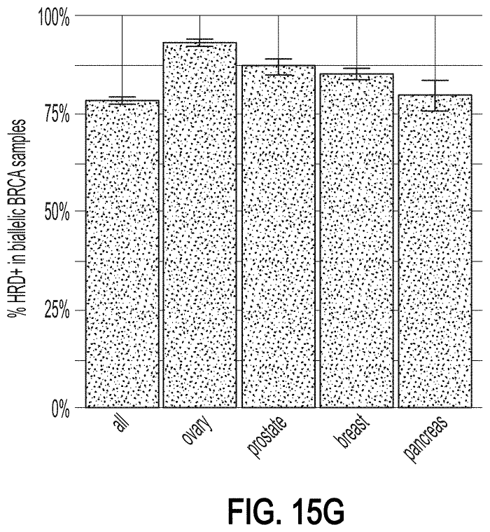

HRDsig performance in the test dataset was evaluated using receiver operating characteristic (ROC) plots. Examining ROC-AUC, the HRDsig algorithm outperformed gLOH overall and across diseases in distinguishing biallelic BRCA1/2 and homologous recombination repair wildtype (HRRwt) samples. Sensitivity to detect biallelic BRCA1/2 alterations was high across HRD-associated tumors; ovarian cancers (93%), prostate cancers (87%), breast cancers (85%), pancreatic cancers (80%). Patients with BRCA1/2 alterations were used as true positives, and HRRwt samples (negative for 14 HRR genes) were used as true negatives. Performance was compared to % gLOH and GIS scores. FIGS. 15A-15F show ROC curves comparing HRDsig to % gLOH and GIS scores for all samples (FIG. 15A), prostate cancer (FIG. 15B), pancreatic cancer (FIG. 15C), ovarian cancer (FIG. 15D), breast cancer (FIG. 15E), and all other samples (FIG. 15F). FIG. 15G shows the fraction of biallelic samples that were called as HRDsig-positive in all samples, ovarian cancer, prostate cancer, breast cancer, and pancreatic cancer.

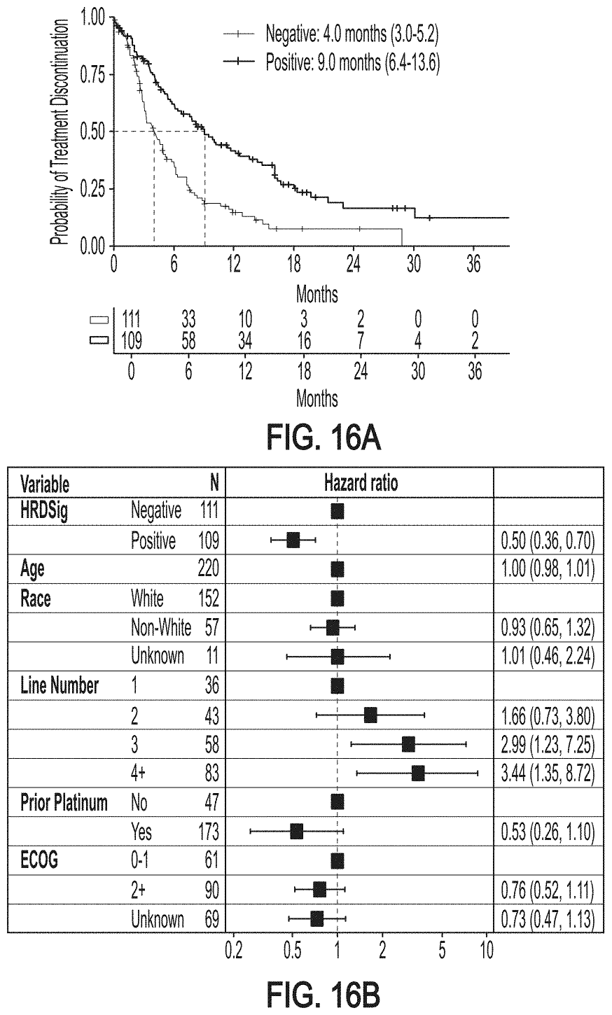

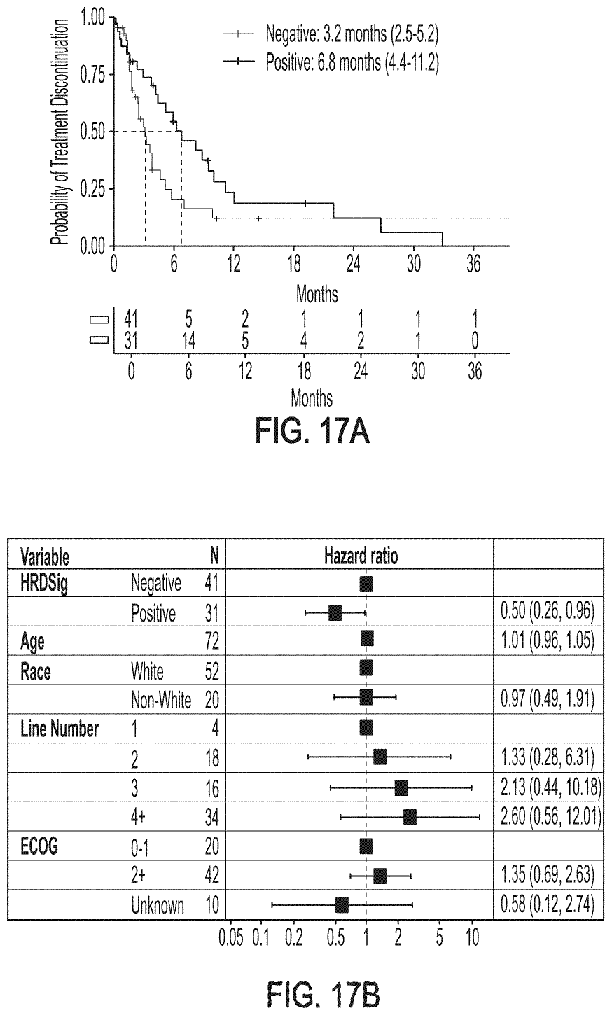

HRDsig predicts PARPi benefit in Ovarian and Prostate clinical cohorts. The real-world clinico-genomic database (CGDB) was leveraged to examine the association of HRDsig positivity with outcomes on PARPi for patients with ovarian cancer (FIGS. 16A-16B). 220 patients were treated with PARPi following CGP specimen collection (HRDsig-positive=109; negative=111). HRDsig positivity was associated with improved time to treatment discontinuation (TTD; median 9.0 months versus 4.0 months; multivariate HR=0.50 [0.36-0.70], p<0.001). In a prostate CGDB, 72 metastatic castration-resistant prostate cancer (mCRPC) patients (FIGS. 17A-17B) were treated with PARPi (HRDsig-positive=31; negative=41). HRDsig was significantly associated with prolonged TTD on PARPi (median 6.8 months versus 3.2 months; HR=0.50 [0.26-0.96], p=0.036).

HRDsig was also able to predict PARPi benefit in BRCAwt ovarian cancer. HRDsig positivity was associated with improved time to treatment discontinuation (TTD median 5.4 months [3.0-4.9) versus 5.4 months [4.3-9.5]; multvariate HR=0.67 [0.44-1.02]).

Genome-wide LOH and genome instability are currently utilized in ovarian cancer for identifying patients who may benefit from PARPi. Therefore the performance of these measures was examined, as measured in our test stratifying PARPi TTD in ovarian cancer. gLOH (≥16%) and GIS (≥42) were associated with significant PARPi TTD (gLOH HR 0.49 [0.33-0.72] p<0.001; GIS HR 0.69 [0.50-0.97] p=0.03). Further, gLOH (>8.29%) and GIS (≥42) were examined in prostate cancer. While both gLOH and GIS identified fewer patients than HRDsig (gLOH: 22 v 31; GIS: 14 v 31), each trended predictive for PARPi TTD (gLOH-high: HR=0.53 [0.24-1.18] p=0.3; GIS>42: HR=0.44 [0.17-1.09], p=0.08).

PARPi treatment improves outcomes in HR deficient patients in a number of tumor types. Current patient selection strategies primarily rely on the presence of germline and/or tumor alterations in BRCA1 and BRCA2. These strategies have been successful in ovarian, prostate, pancreatic, and breast cancer; however, beyond these BRCA-associated cancer types, this biomarker strategy may have pitfalls. BRCA1/2 alterations, even those of germline origin, may not contribute to the pathogenicity of diseases such as lung cancer or colorectal cancer. Furthermore, in diseases with high mutational burden, these BRCA1/2 alterations may represent monoallelic passenger alterations. Ultimately, a mutation-only approach poses a diagnostic challenge for PARPi selection.

Potential alternative approaches involve assessing HRD through RAD51 foci formation or through quantification of copy number scarring. While successful in ovarian cancer, it was not previously clear if these biomarkers would be more broadly applicable across tumor types. In particular, gLOH as well as GIS scores exhibited relatively poor ability to separate biallelic BRCA1/2 mutant and wildtype samples in prostate cancer. A more nuanced biomarker may be required for robust prediction of HRD across tumor types.

Described herein is an HRD signature trained with a diverse set of copy number and indel features. While the highest rates of positivity were seen in fallopian tube, ovarian, peritoneal, breast, and prostate cancer, appreciable frequencies were seen across tumor types, with a 6% overall prevalence. HRDsig that was associated with biallelic BRCA1/2 alterations was high across tumor types. Importantly, HRDsig identified a BRCA1/2 wildtype population enriched for biallelic alterations in other HRR genes and in some cases lacking any HRR alteration, suggesting that HRDsig is able to detect a BRCAness population not currently eligible for PARPi treatment in most indications. In ovarian and prostate cancer clinical cohorts, HRDsig was predictive of improved outcomes on PARPi, with similar or trending superior performance relative to gLOH and GIS. In ovarian cancer, sample numbers were sufficient to demonstrate predictive power of HRDsig in the BRCAwt population. These findings suggest that HRDsig has potential utility in selecting a broader population of patients that may benefit from PARPi use. The results in prostate cancer suggest that HRD signatures may have utility in predicting PARPi response beyond ovarian cancer.

Characterization of pan-cancer copy number signatures revealed distinct molecular subtypes across a wide array of cancers. Many of these signatures were present in almost all tumor types, reflecting an opportunity for the use of pattern-based scores in targeted therapy selection in pan-cancer trials. Even for HRD, which represents a well appreciated and targeted phenotype, it was found that patterns of scarring differed across tumor types with prostate-specific patterns, which requires a nuanced genomic scar-based signature. The algorithm was developed using a hybrid capture targeted panel and may be applicable to other panel-based tests including liquid biopsies, which would be valuable in cases where limited tissue is available, or a post-progression sample is preferred.

### Example 2— Analysis of HRD Signature Predicts PARP Inhibitor Response in Breast Cancer

This Example describes a study that used a nationwide (US-based, —280 US cancer clinics) de-identified clinico-genomic database to evaluate the predictive power of HRDsig for metastatic breast cancer (mBC) patients. Outcomes of 188 evaluable mBC patients treated with PARPi were examined. Homologous recombination (HR) mutations were defined as pathogenic alterations in BRCA1, BRCA2, ATM, BARD1, BRIP1, CDK12, CHEK1, CHEK2, FANCL, PALB2, RAD51B, RAD51C, RAD51D and RAD54L. Mutation germline status was classified as somatic or germline using a somatic, germline, zygosity algorithm (Sun et al.) GIS was calculated using segmented copy number profiles. Real-world overall survival (rwOS), accounting for left-truncation, and real-world progression free survival (rwPFS) from start of PARPi were estimated using Kaplan-Meier analysis. Cox proportional hazards models were adjusted (aHR) for age, race, receptor subtype, performance status (ECOG) near start of therapy, prior platinum, line number, and CNS involvement.

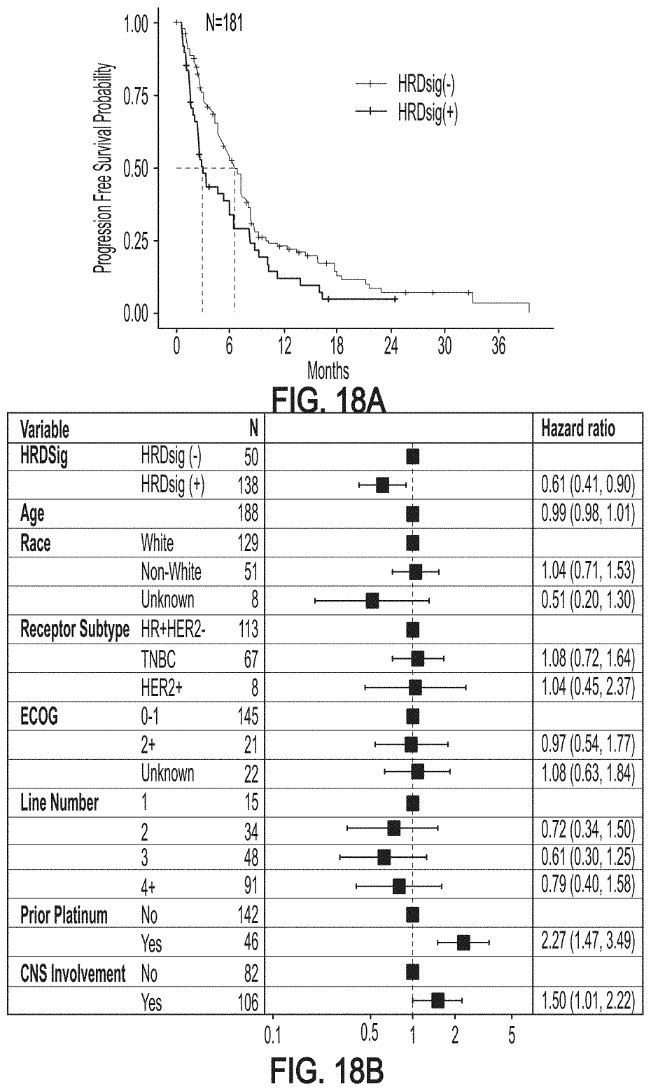

Amongst all 188 evaluable mBC patients treated with PARPi, HRDsig(+) was detected in 138 (73%). Germline BRCA (gBRCA), somatic BRCA (sBRCA), and germline PALB2 (gPALB2) tumors were 88% (70/80), 70% (16/23), and 75% (6/8) HRDsig(+), respectively. For patients with BRCA1/2 alterations with unknown germline status, other HR alterations, or HR wildtype 85% (34/40), 39% (7/18), and 26% (5/19) were HRDsig(+), respectively. Differences in characteristics between HRDsig(+) and HRDsig(−) patients are presented in Table 1. For patients with gBRCA1/2 mutations, real-world progression free survival (rwPFS) and real-world overall survival (rwOS) were 6.3 and 16.2 months. HRDsig(+) was associated with longer rwPFS (6.3 months vs 2.8 months; aHR 0.61 [0.41-0.90], p=0.01; FIG. 18A) HRDsig(+) but not with a significant improvement in rwOS (17.8 vs 13.0 months, aHR 0.70 [0.44-1.10], p=0.12). See FIG. 18B, showing the results from a multivariate analysis of predictors of response in breast cancer, according to some embodiments.

HRDsig(+) is common in mBC patients whose tumors harbor BRCA1/2 and gPALB2 mutations but also observed in tumors with other HR alterations and even those that are HR wildtype. Using a real-world clinical dataset, HRDsig positivity was found to be associated with benefit on PARPi. This data supports rationale for randomized clinical trials in mBC beyond the gBRCA population assessing HRDsig for association with PARPi outcomes.

TABLE 1

| | |
|---|---|
| HRDsig(+) | HRDsig(−) |
| N = 138 | N = 50 |

| | | | | |
|---|---|---|---|---|
| Race: | | | | |
| White, n (%) | 88 | (64) | 41 | (82) |
| Non-white, n (%) | 43 | (31) | 8 | (16) |
| Unknown, n (%) | 7 | (5) | 1 | (2) |
| Age, median [IQR] | 55.5 | [46-65] | 58 | [47.25-64] |
| Subtype: | | | | |
| HR+ HER2−, n (%) | 77 | (56) | 36 | (71) |
| HER2+, n (%) | 7 | (5) | 1 | (4) |
| TNBC, n (%) | 54 | (39) | 13 | (25) |
| ECOG: | | | | |
| 0-1, n (%) | 103 | (75) | 42 | (84) |
| 2+, n (%) | 20 | (15) | 1 | (2) |
| Unknown, n (%) | 15 | (11) | 7 | (14) |
| Prior Platinum: | | | | |
| Yes, n (%) | 37 | (27) | 9 | (18) |
| No, n (%) | 101 | (73) | 41 | (82) |
| Line number: | | | | |
| 1, n (%) | 11 | (8) | 4 | (8) |
| 2, n (%) | 31 | (23) | 3 | (6) |
| 3, n (%) | 33 | (24) | 15 | (30) |
| 4+, n (%) | 63 | (46) | 28 | (56) |
| CNS involvement: | | | | |
| Yes, n (%) | 75 | (54) | 31 | (62) |
| No, n (%) | 63 | (46) | 19 | (38) |

### Example 3—HRD Signature Positivity is a Novel Biomarker of First Line FOLFIRINOX Benefit in Pancreatic Cancer Patients

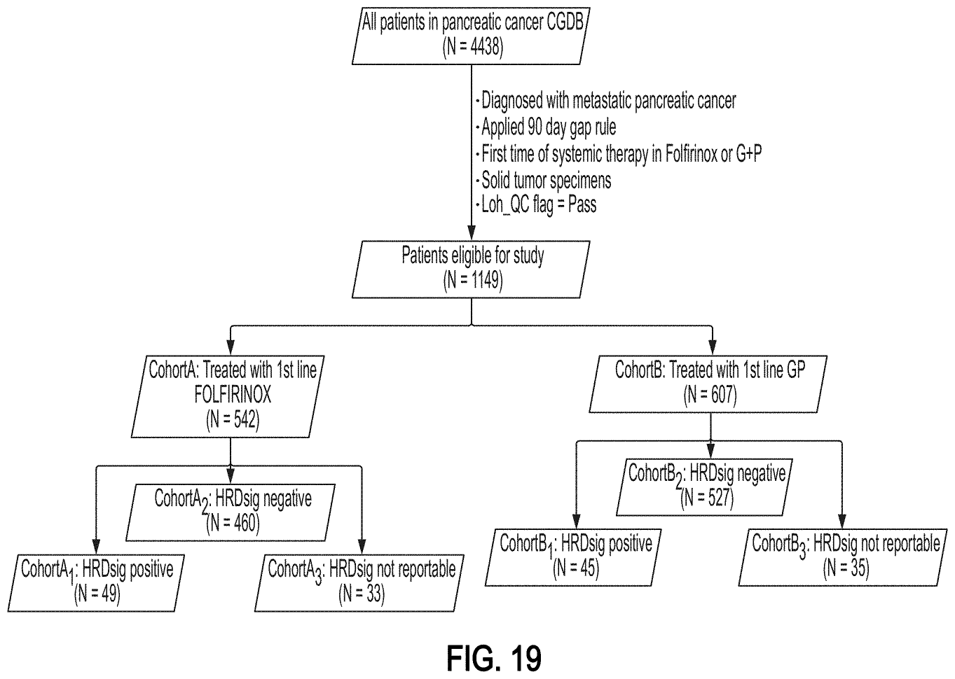

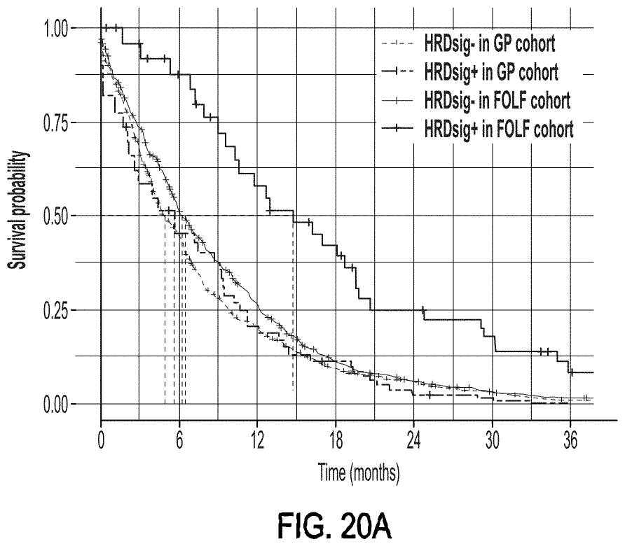

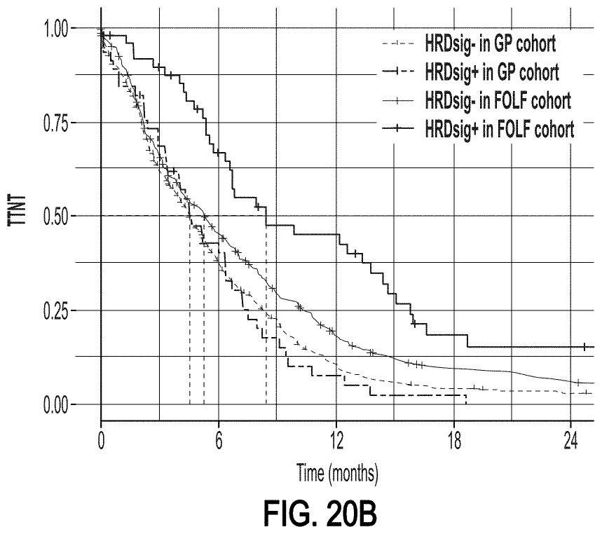

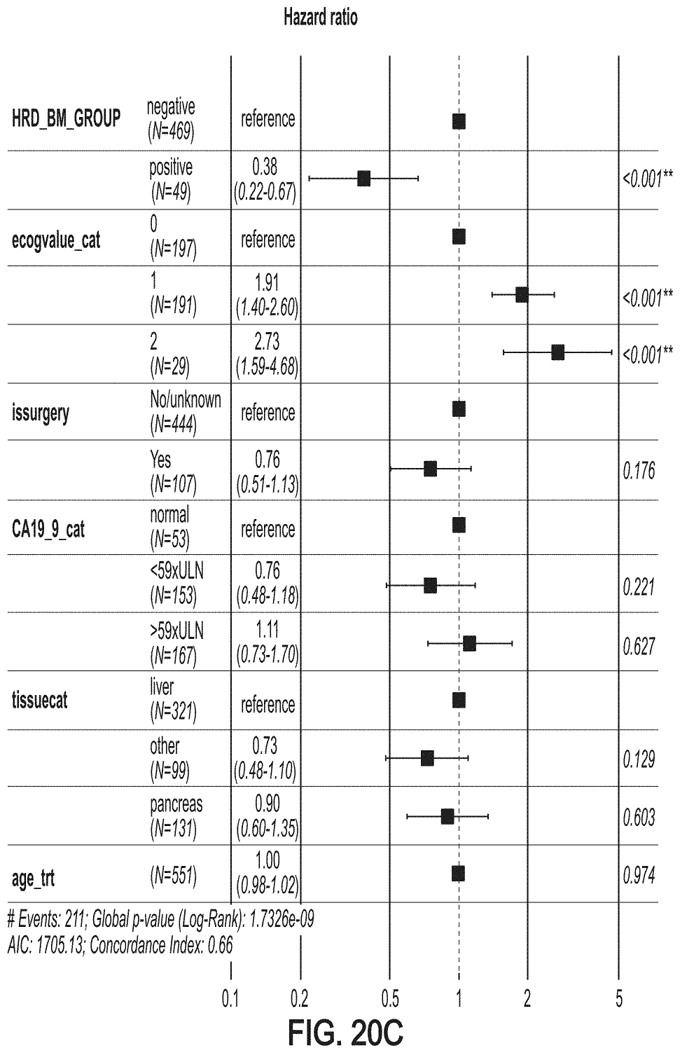

Pancreatic cancer is the fourth leading cause of cancer deaths with a 5-year survival of only 11%. FOLFIRINOX (FOLF; leucovorin, fluorouracil, irinotecan, oxaliplatin) or GP (gemcitabine with albumin-bound paclitaxel) are standard in the first line (1L) setting, although no biomarkers are available to guide therapy selection. This Example describes a study that used a nationwide (US-based, ˜280 US cancer clinics) de-identified pancreatic cancer clinico-genomic database (CGDB) (FIG. 19). Outcomes of 1149 evaluable metastatic pancreatic cancer patients treated with 1L FOLF or GP were examined. HRDsig was called using a machine-learning based algorithm. Real-world overall survival (rwOS), accounting for left-truncation from start of 1L, was estimated using Kaplan-Meier analysis (FIG. 20A). Cox proportional hazards models were adjusted for age, surgery, ECOG, CA19-9, and tissue type with Random Sample Imputation for missing clinical values (FIG. 20C). Tables 2 and 3 below outline clinical features of the pancreatic patients in this study.

TABLE 2
Clinical features of HRDsig(+) and HRDsig(−) pancreatic
cancer patients.

| | HRDSIG+ | | HRDSIG− | | |
|---|---|---|---|---|---|
| | (N = 94) | | (N = 987) | | P-VALUE |
| AGE AT TREATMENT | 67 | (33-83) | 66 | (30-95) | 0.611 |
| START, YEARS, | | | | | |
| MEDIAN (RANGE) | | | | | |
| 1ST LINE TREATMENT | | | | | 0.359 |
| FOLF | 49 | (52.1) | 460 | (46.6) | |
| GP | 45 | (47.9) | 527 | (53.4) | |
| SEX | | | | | 0.436 |
| FEMALE | 46 | (48.9) | 436 | (44.2) | |
| MALE | 48 | (51.1) | 551 | (55.8) | |
| RACE | | | | | 0.426 |
| WHITE | 70 | (74.5) | 648 | (65.7) | |
| BLACK OR AFRICAN | 4 | (4.3) | 80 | (8.1) | |
| AMERICAN | | | | | |
| ASIAN | 1 | (1.1) | 18 | (1.8) | |
| OTHER RACE | 14 | (17.9) | 156 | (15.8) | |
| MISSING | 5 | (5.3) | 85 | (8.6) | |
| ADVANCED STAGE AT | | | | | 0.185 |
| DIAGNOSIS (IV) | | | | | |
| YES | 72 | (76.6) | 712 | (72.1) | |
| NO | 16 | (17.0) | 237 | (24.0) | |
| UNKNOWN/NOT | 6 | (6.4) | 38 | (3.9) | |
| DOCUMENTED | | | | | |
| SURGERY | | | | | 0.3788 |
| YES | 15 | (17.0) | 212 | (21.5) | |
| NO/UNKNOWN | 78 | (83.0) | 775 | (78.5) | |
| ECOG | | | | | 0.31 |
| 0 | 34 | (36.2) | 296 | (30.0) | |
| 1 | 28 | (29.8) | 366 | (37.1) | |
| >=2 | 8 | (8.5) | 84 | (8.5) | |
| MISSING | 24 | (25.5) | 241 | (24.4) | |
| CA19-9 | | | | | 0.256 |
| NORMAL | 5 | (5.3) | 99 | (10) | |
| <59XULN | 32 | (34.0) | 286 | (29.0) | |
| >59XULN | 27 | (28.7) | 285 | (28.9) | |
| MISSING | 30 | (31.9) | 317 | (32.1) | |
| PRIMARY SITE | | | | | 0.628 |
| BODY | 20 | (21.3) | 199 | (20.2) | |
| HEAD | 43 | (45.7) | 424 | (43.0) | |
| TAIL | 20 | (21.3) | 235 | (23.8) | |
| OVERLAPPING SITES | 11 | (11.7) | 107 | (10.8) | |
| PANCREAS, NOS | 0 | (0.0) | 22 | (2.2) | |
| PRACTICE TYPE | | | | | 0.028 |
| ACADEMIC | 17 | (18.1) | 100 | (10.1) | |
| COMMUNITY | 77 | (81.9) | 887 | (89.9) | |
| TISSUE OF ORIGIN | | | | | <0.001 |
| LIVER | 71 | (75.5) | 496 | (50.3) | |
| OTHER | 8 | (8.5) | 193 | (19.6) | |
| PANCREAS | 15 | (16.0) | 298 | (30.2) | |
| TUMOR TYPE | | | | | 0.099 |
| PANCREAS | 12 | (12.8) | 89 | (9.0) | |
| CARCINOMA (NOS) | | | | | |
| PANCREAS DUCTAL | 69 | (73.4) | 748 | (75.8) | |
| ADENOCARCINOMA | | | | | |
| PANCREATOBILIARY | 10 | (10.6) | 66 | (6.7) | |
| CARCINOMA | | | | | |
| OTHER | 3 | (3.2) | 84 | (8.5) | |
| SMOKING STATUS | | | | | 0.9531 |
| HISTORY OF | 50 | (53.2) | 526 | (53.3) | |
| SMOKING | | | | | |
| NO HISTORY | 44 | (46.8) | 460 | (46.6) | |
| OF SMOKING | | | | | |
| UNKNOWN/NOT | 0 | (0.0) | 1 | (0.1) | |
| DOCUMENTED | | | | | |

TABLE 3
Clinical features of PC patients treated with FOLFIRINOX or GP.

| | FOLF | | GP | | |
|---|---|---|---|---|---|
| | (N = 542) | | (N = 607) | | P-VALUE |
| AGE AT TREATMENT | 63 | (30-83) | 69 | (32-85) | <0.001 |
| START, YEARS, | | | | | |
| MEDIAN (RANGE) | | | | | |
| HRDSIG | | | | | 0.36 |
| POSITIVE | 49 | (9.0) | 45 | (7.4) | |
| NEGATIVE | 460 | (84.9) | 527 | (86.8) | |
| NOT REPORTABLE | 33 | (6.1) | 35 | (5.8) | |
| SEX | | | | | 0.037 |
| FEMALE | 223 | (41.1) | 288 | (47.4) | |
| MALE | 319 | (58.9) | 319 | (52.6) | |
| RACE | | | | | 0.47 |
| WHITE | 349 | (64.4) | 415 | (68.4) | |
| BLACK OR AFRICAN | 43 | (7.9) | 48 | (7.9) | |
| AMERICAN | | | | | |
| ASIAN | 9 | (1.7) | 10 | (1.6) | |
| OTHER RACE | 94 | (17.3) | 86 | (14.2) | |
| MISSING | 47 | (8.7) | 48 | (7.9) | |
| ADVANCED STAGE AT | | | | | <0.001 |
| DIAGNOSIS (IV) | | | | | |
| YES | 433 | (79.9) | 398 | (65.6) | |
| NO | 96 | (17.7) | 176 | (29.0) | |
| UNKNOWN/NOT | 13 | (2.4) | 33 | (5.4) | |
| DOCUMENTED | | | | | |
| SURGERY | | | | | 0.059 |
| YES | 102 | (18.8) | 143 | (23.6) | |
| NO/UNKNOWN | 440 | (81.2) | 464 | (76.4) | |
| ECOG | | | | | <0.001 |
| 0 | 194 | (35.8) | 162 | (26.7) | |
| 1 | 190 | (35.1) | 230 | (37.9) | |
| >=2 | 29 | (5.4) | 68 | (11.2) | |
| MISSING | 129 | (23.8) | 147 | (24.2) | |
| CA19-9 | | | | | 0.32 |
| NORMAL | 52 | (9.6) | 58 | (9.6) | |
| <59XULN | 148 | (27.3) | 193 | (31.8) | |
| >59XULN | 165 | (30.4) | 171 | (28.2) | |
| MISSING | 177 | (32.7) | 185 | (30.5) | |
| PRIMARY SITE | | | | | 0.22 |
| BODY | 114 | (21.0) | 121 | (19.9) | |
| HEAD | 226 | (41.7) | 276 | (45.5) | |
| TAIL | 141 | (26.0) | 127 | (20.9) | |
| OVERLAPPING | 51 | (9.4) | 71 | (11.7) | |
| SITES | | | | | |
| PANCREAS, NOS | 10 | (1.8) | 12 | (2.0) | |
| PRACTICE TYPE | | | | | 0.035 |
| ACADEMIC | 67 | (12.4) | 51 | (8.4) | |
| COMMUNITY | 475 | (87.6) | 556 | (91.6) | |
| TISSUE OF | | | | | 0.002 |
| ORIGIN | | | | | |
| LIVER | 316 | (58.3) | 295 | (48.6) | |
| OTHER | 95 | (17.5) | 114 | (18.8) | |
| PANCREAS | 131 | (24.2) | 198 | (32.6) | |
| TUMOR TYPE | | | | | 0.61 |
| PANCREAS | 44 | (8.1) | 63 | (10.4) | |
| CARCINOMA (NOS) | | | | | |
| PANCREAS DUCTAL | 415 | (76.6) | 450 | (74.1) | |
| ADENOCARCINOMA | | | | | |
| PANCREATOBILIARY | 38 | (7.0) | 44 | (7.2) | |
| CARCINOMA | | | | | |
| OTHER | 45 | (8.3) | 50 | (8.2) | |
| SMOKING STATUS | | | | | 0.47 |
| HISTORY OF | 284 | (52.4) | 330 | (54.4) | |
| SMOKING | | | | | |
| NO HISTORY OF | 257 | (47.4) | 277 | (45.6) | |
| SMOKING | | | | | |
| UNKNOWN/NOT | 1 | (0.2) | 0 | (0.0) | |
| DOCUMENTED | | | | | |

HRDsig positivity was observed in 9% (94/1081) of PC patients. 52% received FOLF and 48% received GP in the 1L setting. FOLF treated patients with HRDsig(+) had better real world Overall Survival (rwOS; FIG. 20A) and time to next treatment (TTNT; FIG. 20B) compared to those with HRDsig(−) (median rwOS 14.8 vs 6.3 months; aHR: 0.50 (0.34-0.73), p<0.001; TTNT 8.48 vs 4.57 mo; aHR: 0.37 (0.22-0.63), p<0.001). In contrast, minimal benefit was observed for FOLF relative to GP in the HRDsig(−) population (median rwOS 6.28 vs 5.06 months; aHR: 0.86 (0.74-1.00), p=0.05; TTNT 5.29 vs 4.57 months; aHR: 0.76 (0.66-0.88), p<0.001). Real world OS at 1- and 2-years was 58% and 25% in FOLF-treated HRDsig(+) patients, relative to 21% and 2% in GP-treated HRDsig(+) patients. Minimal differences were observed in rwOS for HRDsig(−) patients treated with FOLF and GP (median 6.3 months v 5.1 months). Thus, HRDsig positivity was predictive of FOLF (e.g. platinum-containing) treatment benefit in metastatic pancreatic cancer patients.

These results demonstrate that HRDsig is predictive of significant benefit for pancreatic cancer patients on platinum-containing FOLFIRINOX relative to GP while also showing minimal added benefit from FOLF treatment for pancreatic cancer patients with HRDsig(−). This is important because the choice of 1L standard of care in PC is challenging. In patients with a good prognosis, the decision to prescribe GP or FOLF comes with trade-offs. While some patients will obtain significant benefit from FOLF, high-grade toxicities including neutropenia, fatigue, vomiting, and diarrhea occur frequently with potential negative impact on patient quality of life. Biomarkers to identify patients for whom FOLF treatment would have minimal efficacy are also valuable: as shown above, HRDsig(−) patients obtained minimal benefit from FOLF vs GP (median rwOS 6.3 vs 5.1 mo; p=0.05), suggesting that GP may be a more favorable treatment option in these patients to minimize toxicities and toxicity-induced decreases in quality of life. Conversely, the data described above showed significant benefit for HRDsig(+) PC patients treated with FOLF rather than GP, indicating this is a population wherein, when FOLF can be tolerated, wherein this treatment may be preferable.

### Example 4—HRD Signature Positivity is a Novel Biomarker of First Line Platinum-Containing Therapeutic Benefit in Cancer Patients

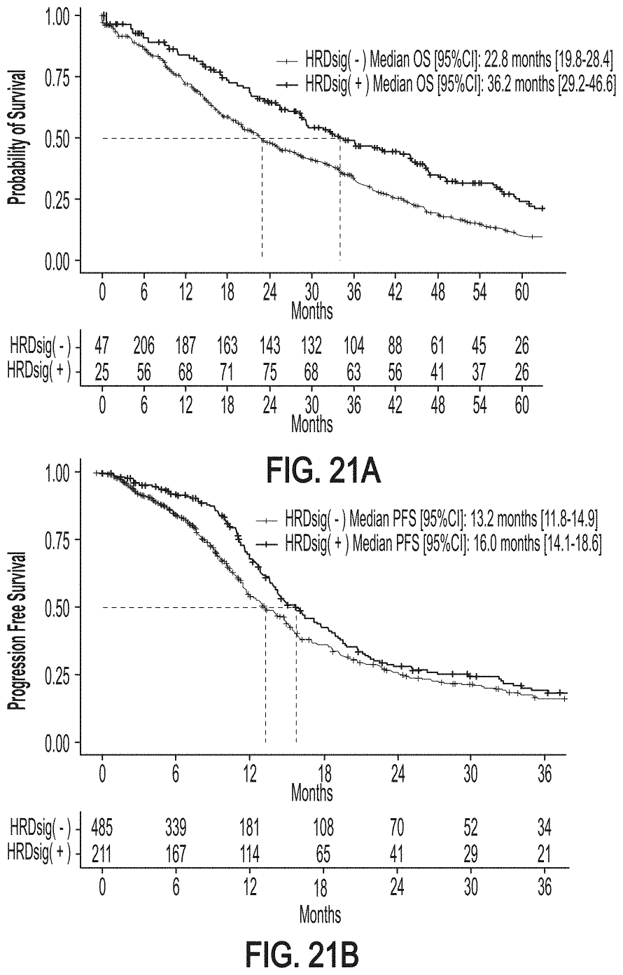

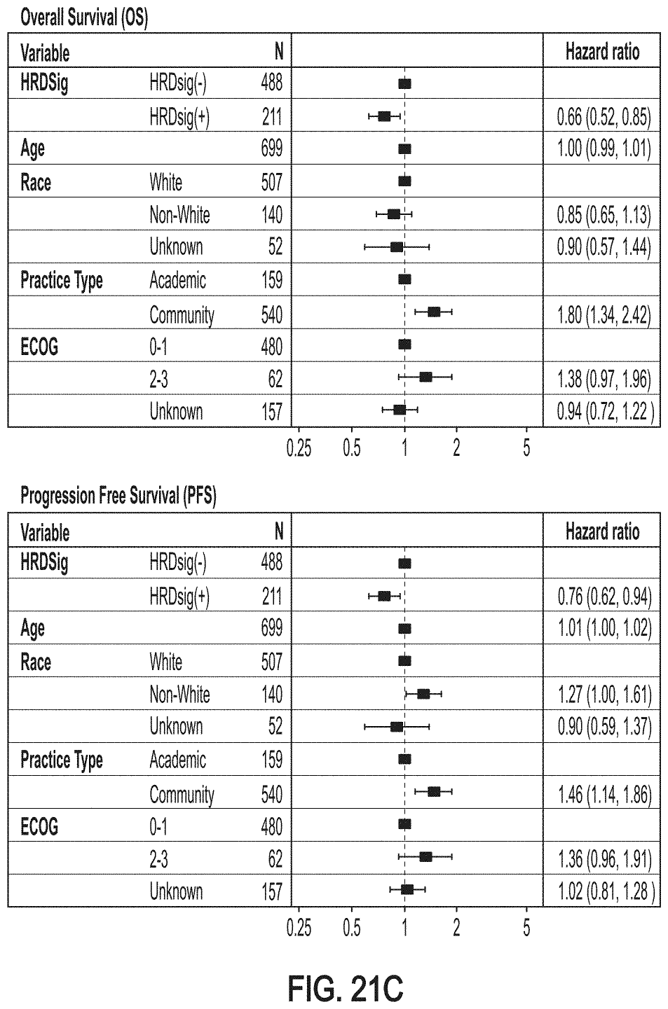

Patients with ovarian cancer, non-small cell lung cancer (NSCLC), or endometrial cancer were assessed for HRDsig and prognosis following first line platinum-containing therapy. FIG. 21A demonstrates that HRDsig positivity in ovarian cancer patients was associated with first line carboplatin +/− Paclitaxel/docetaxel treatment efficacy, as calculated based on the probability of real-world overall survival (rwOS) (Median OS [95% CI]: HRDsig(+) 34.5 [28.5-46.2]; HRDsig(−) 22.5 [19.2-27.7]). FIG. 21B confirms this association by calculating real-world progression-free survival (rwPFS) (Median rwPFS [95% CI]: HRDsig(+) 15.8 [11.8-14.9]; HRDsig(−) 13.1 [11.8-14.9]). Cox proportional hazards models were adjusted for age, race, ECOG, and HRDsig (FIG. 21C) for rwOS (top panel) and rwPFS (bottom panel).

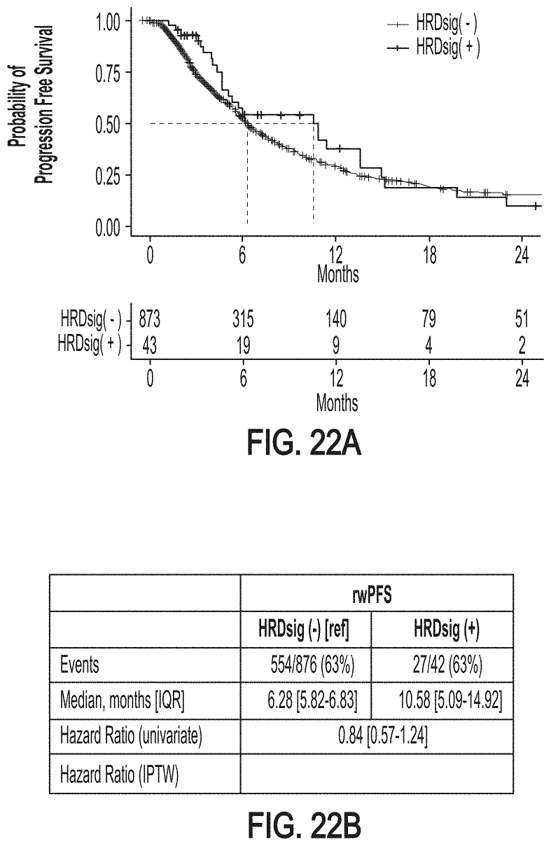

Real-world progression-free survival (rwPFS) was estimated using Kaplan-Meier analysis for patients with NSCLC (FIG. 22A). Cox proportional hazards models multivariate analysis predicted association between HRDsig positivity and treatment efficacy of first-line platinum-containing therapy (FIG. 22B).

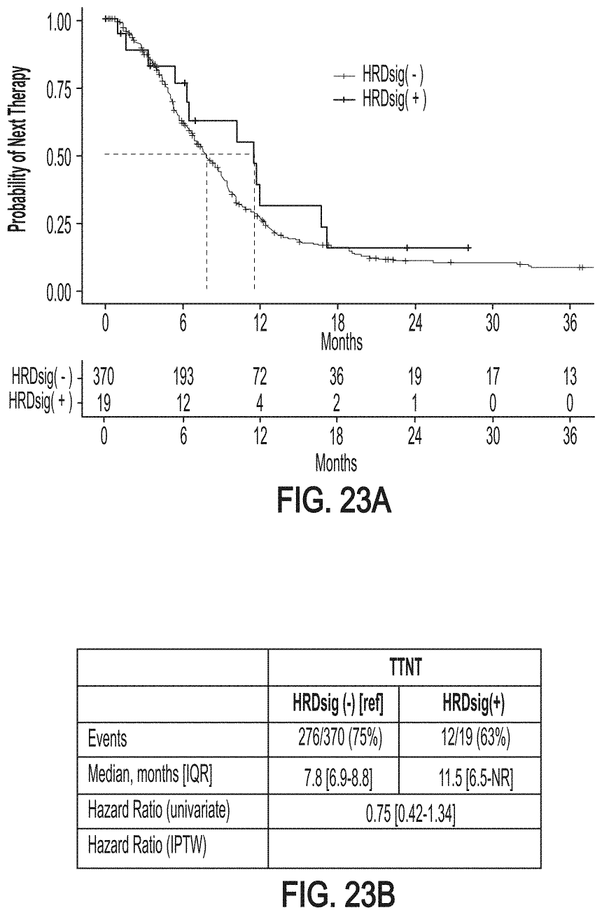

Probability of next therapy was estimated using Kaplan-Meier analysis for patients with endometrial cancer (FIG. 23A). Cox proportional hazards models multivariate analysis predicted association between HRDsig positivity and treatment efficacy of first-line platinum-containing therapy (FIG. 23B). Together, these results demonstrate that HRDsig is predictive of significant benefit for cancer patients receiving first-line platinum-containing therapies.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

Although the disclosure has been fully described with reference to the accompanying figures, it is to be noted that various changes and modifications will become apparent to those skilled in the art. Such changes and modifications are to be understood as being included within the scope of the disclosure as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

1. (canceled)

2. A method, comprising:
receiving, by one or more processors, a plurality of features;
identifying, by the one or more processors, a subset of features in the plurality of features using one or more feature importance metrics; and
training, by the one or more processors, a homologous recombination deficiency (HRD) model based on the identified subset of the plurality of features, wherein the HRD model is configured to receive sample data associated with a genome of a tumor in a subject and identify the tumor in the subject as HRD-positive or HRD-negative using the sample data.

3. A method, comprising:
receiving, by one or more processors, sample data associated with a genome of a tumor in a subject;
inputting, by the one or more processors, the sample data into a trained homologous recombination deficiency (HRD) model, wherein the HRD model is trained by:
determining one or more feature importance metrics associated with each feature of a plurality of features,
identifying a subset of features in the plurality of features using the one or more feature importance metrics, and
training, by the one or more processors, the HRD model based on the identified subset of features; and
classifying, by the one or more processors, using the trained HRD model, the tumor as HRD-positive or HRD-negative.

4. The method of claim 3, wherein the plurality of features comprises one or more copy number features, one or more short variant features, or a combination thereof.

5. The method of claim 3, wherein the one or more feature importance metrics comprise one or more of a Chi-Square test, analysis of variance (ANOVA), random forest, or gradient boosting.

6. The method of claim 3, wherein identifying the subset of features in the plurality of features comprises:
obtaining, by the one or more processors, one or more feature rankings according to the one or more feature importance metrics; and
selecting, by the one or more processors, the subset of the plurality of features based on one or more feature rankings.

7. The method of claim 3, wherein identifying the subset of the plurality of features comprises:
(a) obtaining, by one or more processors, a feature ranking of the plurality of features according to a feature importance metric;
(b) obtaining, by the one or more processors, a new feature set by adding one or more features from the plurality of features to an existing feature set based on the feature ranking;
(c) training, by the one or more processors, a new HRD model using the new feature set;
(d) evaluating, by the one or more processors, the trained new HRD model to obtain an evaluation result; and
(e) storing, by the one or more processors, the evaluation result associated with the new HRD model and the new feature set;
(f) repeating, by the one or more processors, steps (b)-(e) to obtain a plurality of evaluation results until a condition is met; and
(g) selecting, by the one or more processors, the subset of the plurality of features based on the plurality of evaluation results.

8. (canceled)

9. The method of claim 3, wherein the classifying comprises determining at least one of a HRD-positive likelihood score and a HRD-negative likelihood score.

10. (canceled)

11. The method of claim 9, comprising recording, in a digital electronic file associated with the subject, at least one of the HRD-positive likelihood score and the HRD-negative likelihood score.

12. The method of 9, comprising recording, in a digital electronic file associated with the subject, a designation that the tumor is HRD-positive based on the HRD-positive likelihood score or a designation that the tumor is HRD-negative based on the HRD-negative likelihood score.

13. The method of claim 3, wherein the plurality of features comprise at least one of a segment minor allele frequency (segMAF) feature, a number of sequencing reads feature, a segment size feature, a breakpoint count per x megabases feature, a change point copy number feature, a segment copy number feature, a breakpoint count per chromosome arm feature, or a number of segments with oscillating copy number feature.

14-45. (canceled)

46. The method of claim 3, wherein training the HRD model comprises:
receiving, by the one or more processors, an HRD-positive training dataset, wherein the HRD-positive training dataset comprises a plurality of features associated with an HRD-positive tumor and an HRD-positive label;
receiving, by the one or more processors, an HRD-negative training dataset, wherein the HRD-negative training dataset comprises a plurality of features associated with an HRD-negative tumor and an HRD-negative label;
training, by the one or more processors, the HRD model using the HRD-positive training dataset and the HRD-negative training dataset.

47-51. (canceled)

52. The method of claim 3, wherein the tumor in the subject is a prostate cancer, ovarian cancer, breast cancer, non-small cell lung cancer (NSCLC), colorectal cancer (CRC), fallopian tube cancer, endometrial cancer, or pancreatic cancer.

53-65. (canceled)

66. A method of treating a tumor in a subject, comprising:
(a) identifying the tumor as HRD-positive or HRD-negative according to the method of claim 3; and
(b) administering to the subject a therapeutically effective amount of a therapy effective in a HRD-positive tumor if the tumor of the tumor is assessed as HRD positive.

67. The method of claim 66, wherein the therapy effective in a HRD positive tumor comprises a platinum-based chemotherapeutic agent or a PARP inhibitor.

68. The method of claim 66 comprising administering to the subject a therapeutically effective amount of a therapy that does not comprise a platinum-based chemotherapeutic agent or a PARP inhibitor if the tumor is assessed as HRD negative.

69. A method for selecting a therapy for a tumor in a subject, the method comprising:
(a) assessing the tumor as HRD-positive or HRD-negative according to the method of claim 3; and
(b) selecting a therapy that is effective in a HRD-positive tumor if the tumor is assessed as HRD positive.

70. The method of claim 69, comprising selecting a therapy that does not comprise a platinum-based drug or a PARP inhibitor if the tumor is assessed as HRD negative.

71. The method of claim 70, wherein the therapy that is effective in a HRD positive tumor comprises a platinum-based chemotherapeutic agent or a PARP inhibitor.

72-74. (canceled)

75. A method of treating a subject having a cancer with a therapy comprising a platinum-based chemotherapeutic agent or a PARP inhibitor, comprising:
determining a homologous recombination deficient (HRD) status of a sample obtained from the subject, and
administering the platinum-based chemotherapeutic agent or the PARP inhibitor to the subject if the HRD status of the sample is determined to be HRD-positive.

76-80. (canceled)

81. A method of treating a homologous recombination deficient (HRD)-positive cancer in a subject, comprising:
identifying the cancer as an HRD-positive cancer, comprising:
obtaining genomic data comprising values for a plurality of genomic features for the cancer;
inputting, by one or more processors, the genomic data into a trained HRD model configured to characterize the cancer as HRD-positive or HRD-negative based on the genomic data; and
characterizing, by the one or more processors, using the trained HRD model, the cancer as HRD-positive; and
responsive to identifying the cancer as an HRD-positive cancer, administering to the subject a therapy comprising a platinum-based chemotherapeutic agent or a PARP inhibitor.

82-177. (canceled)
